(19) United States
(12) Patent Application Publication
(10) Pub. No.: US 2025/0122291 A1
(43) Pub. Date: Apr. 17, 2025

(54) ANTI-TRKA ANTIBODIES AND USES THEREOF

(71) Applicant: Cephalon LLC, West Chester, PA (US)

(72) Inventors: Teresa Domagala, Alexandria (AU); David Rickards, Broadway (AU); Jennifer Renee Stratton, San Carlos, CA (US); Kimberly Danielle Mackenzie, Mountain View, CA (US); Eric C. Beattie, Brisbane, CA (US); Adam Clarke, Riverstone (AU)

(21) Appl. No.: 18/913,217

(22) Filed: Oct. 11, 2024

Related U.S. Application Data
(60) Provisional application No. 63/590,132, filed on Oct. 13, 2023.

(51) Int. Cl. C07K 16/28; A61K 39/00; A61P 25/02

(52) U.S. Cl. CPC C07K 16/2863; A61P 25/02; A61K 2039/505; C07K 2317/33; C07K 2317/92

(57) ABSTRACT

Disclosed herein are anti-TrkA antibodies, or antigen-binding fragments thereof, and uses of the antibodies or fragments thereof.

### CROSS REFERENCE TO RELATED APPLICATIONS

This application claims priority to U.S. Provisional Application No. 63/590,132, filed on Oct. 13, 2023, the disclosure of which is hereby incorporated by reference in its entirety.

### SEQUENCE LISTING

The instant application contains a Sequence Listing which is being submitted herewith electronically in XML format and is hereby incorporated by reference in its entirety. Said XML copy, created on Oct. 22, 2024, is named 132937000002_Updated Sequence listingUS.xml and is 98 bytes in size.

### FIELD OF THE INVENTION

Disclosed herein are anti-tropomyosin receptor kinase A (TrkA) antibodies, or antigen-binding fragments thereof, and uses thereof. The disclosed anti-TrkA antibodies, or antigen-binding fragments thereof, bind to and activate TrkA, and are suitable for treating disorders in which neuronal dysfunction is associated with the disease pathology. For example, wherein the neuronal dysfunction is due to impaired neuronal growth, repair, restoration, or regeneration.

### BACKGROUND

Tropomyosin receptor kinase A (TrkA) is a cell surface transmembrane receptor for nerve growth factor (NGF) NGF is the founding member of the neurotrophins, a family of secreted proteins that are involved in the maintenance and survival of neurons in the central and peripheral nervous system.

The TrkA receptor is a 140 kDa transmembrane glycoprotein, and while NGF preferentially binds TrkA, it also binds with low affinity to a p75 receptor whose signaling function is unclear. Homo or heterodimers or oligomers of TrkA and p75 bind NGF with higher affinity suggesting that specific receptor conformations may play specific functions.

The TrkA receptor consists of an extracellular domain, a transmembrane region, and an intracellular tyrosine kinase domain. The extracellular domain contains a cysteine-rich cluster followed by three leucine-rich 24-residue repeats (LRR), another cysteine-rich cluster, and two immunoglobulin-like domains (Ig-C1 and Ig-C2). Out of the five domains in its extracellular portion, the immunoglobulin-like domain proximal to the membrane (Ig-C2) is necessary and sufficient for NGF homodimer binding.

TrkA signaling pathways are initiated when NGF binds to the extracellular Ig-C2 domain of TrkA at the cell surface, which causes TrkA dimerization and leads to autophosphorylation of specific tyrosine residues in the cytoplasmic domain of TrkA. Since microinjection of NGF into cells does not result in NGF biological signals, cell surface receptor ligation and internalization of TrkA or NGF-TrkA complexes mediates these effects.

During early development, NGF has a critical role as a survival factor for neurons, including sensory and sympathetic neurons. NGF null mice have a severe loss of sympathetic and sensory neurons, and animals lacking TrkA receptors show a phenotype similar to NGF null mice, underscoring the importance of NGF-TrkA signaling for the development of these neuronal populations.

Deregulation of NGF synthesis, transport and/or utilization by peripheral nervous system (PNS) neurons may underlie peripheral neuropathies based on evidence obtained in animal models and in human neuropathologies. This evidence formed the rationale for the clinical investigations of systemically administered recombinant human NGF (rhNGF) as a potential therapy for diabetic and HIV-associated peripheral neuropathies. Specifically, phase I clinical trial investigation of rhNGF administration in healthy subjects resulted in side effects, such as injection site pain and myalgias. A phase II clinical trial in diabetic polyneuropathy revealed an improvement of neuropathic symptoms in the NGF-treated patients, but with the occurrence of injection site hyperalgesia, myalgias and arthralgias. In phase III trials similar side effects were noted as the previous studies. Using NGF as a therapeutic has several disadvantages including manufacturability, agonism of both TrkA and p75^{NTR}, short half-life (˜4 hours in monkeys) and tolerability issues related to pain/myalgias.

Like most kinase growth factor receptors, as TrkA signals through receptor dimerization, monovalent TrkA-binding agents are antagonistic or have no biological effects, whereas bivalent receptor-binding agents such as NGF (a homodimer) or antibodies can be agonistic. The principle of using polyclonal antibodies to activate neural receptors has been demonstrated.

Agonism of TrkA was purportedly disclosed in WO1997021732 as having hormone (i.e. NGF-like) effects. Antibodies against TrkA, which antagonize the activity of NGF have also been disclosed. For example, U.S. Pat. No. 9,365,654, Int'l Pub. No. WO2006/131952, Int'l Pub. No. WO2019/168730, and Int'l Pub. No. WO2020/238998 describe anti-TrkA antibodies which block NGF's interaction with the receptor, thereby preventing activation of TrkA, and purportedly providing in vivo analgesic activity.

Discussion or mention of any piece of prior art in this specification is not to be taken as an admission that the prior art is part of the common general knowledge of the skilled addressee of the specification.

### SUMMARY OF INVENTION

Disclosed herein are anti-TrkA antibodies, or antigen-binding fragments thereof, which bind to TrkA and activate TrkA to thereby promote a biological response (e.g. neuron cells survival and differentiation) and/or induction of downstream pathway(s) (i.e. mediated by TrkA signal transduction). The disclosed anti-TrkA antibodies are suitable for treating a disease where neuronal dysfunction is associated with the disease pathology such as, for example, diabetic peripheral neuropathy, chemotherapy-induced peripheral neuropathy, small fiber neuropathy, nerve injury (e.g. traumatic nerve injury or post-surgical nerve injury), wound healing (e.g. diabetic foot ulcers), or optic neuropathies (e.g. glaucoma etc.).

Disclosed herein are anti-tropomyosin receptor kinase A (TrkA) antibodies, or antigen-binding fragments thereof, comprising:
a heavy chain complementarity determining region 1 (HCDR1) comprising the amino acid sequence of SEQ ID NO: 71, a HCDR2 comprising the amino acid sequence of SEQ ID NO: 72, a HCDR3 comprising the amino acid sequence of SEQ ID NO: 73, a light chain complementarity determining region 1 (LCDR1) comprising the amino acid sequence of SEQ ID NO: 74, a LCDR2 comprising the amino acid sequence of SEQ ID NO: 75, and a LCDR3 comprising the amino acid sequence of SEQ ID NO: 76.
wherein the HCDR1 is numbered according to AbM and the remaining CDRs are numbered according to Kabat.

Disclosed herein are anti-TrkA antibodies, or antigen-binding fragments thereof, the antibodies or antigen-binding fragments thereof comprising:
(a) a heavy chain complementarity determining region (HCDR1) comprising the amino acid sequence of SEQ ID NO: 21,
(b) a HCDR2 comprising the amino acid sequence of SEQ ID NO: 22,
(c) a HCDR3 comprising the amino acid sequence of SEQ ID NO: 23;
(d) a light chain complementarity determining region (LCDR1) comprising the amino acid sequence of SEQ ID NO: 24,
(e) a LCDR2 comprising the amino acid sequence of SEQ ID NO: 25, and
(f) a LCDR3 comprising the amino acid sequence of SEQ ID NO: 26.

In an embodiment, in the anti-TrkA antibodies, or antigen-binding fragments thereof:
the Xb residue in SEQ ID NO: 21 is V or I,
the Xc residue in SEQ ID NO: 21 is G or S,
the Xd residue in SEQ ID NO: 22 is T or R,
the Xg residue in SEQ ID NO: 23 is F or W,
the Xh residue in SEQ ID NO: 23 is R or Y,
the X1 residue in SEQ ID NO: 24 is D, G, A, or L,
the X2 residue in SEQ ID NO: 24 is S or A,
the X4 residue in SEQ ID NO: 25 is N or G,
the X5 residue in SEQ ID NO: 25 is N or K,
the X6 residue in SEQ ID NO: 26 is N or A,
the X7 residue in SEQ ID NO: 26 is V, D, or A,
the X8 residue in SEQ ID NO: 26 is S or Y, and
the X9 residue in SEQ ID NO: 26 is G or S.

Thus, disclosed herein are anti-TrkA antibodies, or antigen-binding fragments thereof, wherein the antibodies or antigen-binding fragments thereof comprise:
(a) a heavy chain complementarity determining region (HCDR1) comprising the amino acid sequence of SEQ ID NO: 21,
(b) a HCDR2 comprising the amino acid sequence of SEQ ID NO: 22,
(c) a HCDR3 comprising the amino acid sequence of SEQ ID NO: 23;
(d) a light chain complementarity determining region (LCDR1) comprising the amino acid sequence of SEQ ID NO: 24,
(e) a LCDR2 comprising the amino acid sequence of SEQ ID NO: 25, and
(f) a LCDR3 comprising the amino acid sequence of SEQ ID NO: 26,
wherein:
the Xb residue in SEQ ID NO: 21 is V or I,
the Xc residue in SEQ ID NO: 21 is G or S,
the Xd residue in SEQ ID NO: 22 is T or R,
the Xg residue in SEQ ID NO: 23 is F or W,
the Xh residue in SEQ ID NO: 23 is R or Y,
the X1 residue in SEQ ID NO: 24 is D, G, A, or L,
the X2 residue in SEQ ID NO: 24 is S or A,
the X4 residue in SEQ ID NO: 25 is N or G,
the X5 residue in SEQ ID NO: 25 is N or K,
the X6 residue in SEQ ID NO: 26 is N or A,
the X7 residue in SEQ ID NO: 26 is V, D, or A,
the X8 residue in SEQ ID NO: 26 is S or Y, and
the X9 residue in SEQ ID NO: 26 is G or S.

In an embodiment, in the anti-TrkA antibodies, or antigen-binding fragments thereof:
the Xb residue in SEQ ID NO: 21 is I,
the Xc residue in SEQ ID NO: 21 is S,
the Xd residue in SEQ ID NO: 22 is R,
the Xg residue in SEQ ID NO: 23 is W,
the Xh residue in SEQ ID NO: 23 is Y,
the X1 residue in SEQ ID NO: 24 is D,
the X2 residue in SEQ ID NO: 24 is A,
the X4 residue in SEQ ID NO: 25 is G,
the X5 residue in SEQ ID NO: 25 is N,
the X6 residue in SEQ ID NO: 26 is A,
the X7 residue in SEQ ID NO: 26 is A,
the X8 residue in SEQ ID NO: 26 is S, and
the X9 residue in SEQ ID NO: 26 is S.

In an embodiment, in the anti-TrkA antibodies, or antigen-binding fragments thereof:
the Xb residue in SEQ ID NO: 21 is V,
the Xc residue in SEQ ID NO: 21 is G,
the Xd residue in SEQ ID NO: 22 is T,
the Xg residue in SEQ ID NO: 23 is F,
the Xh residue in SEQ ID NO: 23 is Y,
the X1 residue in SEQ ID NO: 24 is D,
the X2 residue in SEQ ID NO: 24 is S,
the X4 residue in SEQ ID NO: 25 is N,
the X5 residue in SEQ ID NO: 25 is N,
the X6 residue in SEQ ID NO: 26 is N,
the X7 residue in SEQ ID NO: 26 is V,
the X8 residue in SEQ ID NO: 26 is S, and
the X9 residue in SEQ ID NO: 26 is G.

In an embodiment, in the anti-TrkA antibodies, or antigen-binding fragments thereof:
the Xb residue in SEQ ID NO: 21 is I,
the Xc residue in SEQ ID NO: 21 is S,
the Xd residue in SEQ ID NO: 22 is R,
the Xg residue in SEQ ID NO: 23 is W,
the Xh residue in SEQ ID NO: 23 is Y,
the X1 residue in SEQ ID NO: 24 is D,
the X2 residue in SEQ ID NO: 24 is S,
the X4 residue in SEQ ID NO: 25 is N,
the X5 residue in SEQ ID NO: 25 is N,
the X6 residue in SEQ ID NO: 26 is N,
the X7 residue in SEQ ID NO: 26 is D,
the X8 residue in SEQ ID NO: 26 is S, and
the X9 residue in SEQ ID NO: 26 is S.

Also disclosed herein are anti-TrkA antibodies, or antigen-binding fragments thereof, comprising:
(a) a HCDR1, HCDR2 and HCDR3 amino acid sequences of an amino acid sequence of SEQ ID NO:27, 29, 33, 35, 37, 39, 41, 43, 45, 47, 49, 51, 53, 57, 59, 61, 63 or 65, and
(b) a light chain variable region (LCVR) CDR1, CDR2 and CDR3 amino acid sequences derived from an amino acid sequence of SEQ ID NO: 28, 30, 32, 34, 36, 38, 40, 42, 44, 46, 48, 50, 52, 54, 56, 58, 60, 62, 64, or 66,
wherein the CDR sequences are identified by the Kabat, Chothia, AbM or IMGT numbering schemes, or combinations thereof.

In an embodiment, the anti-TrkA antibodies or antigen-binding fragments thereof bind to TrkA and activate TrkA thereby promoting TrkA biological response (e.g. neuron cells survival and differentiation) and/or induction of downstream pathway(s) (i.e. mediated by TrkA signal transduction).

Thus, disclosed herein are anti-TrkA antibodies, or antigen-binding fragments thereof, wherein the antibodies or antigen-binding fragments thereof comprise:
(a) a heavy chain complementarity determining region (HCDR1) comprising the amino acid sequence of SEQ ID NO: 21,
(b) a HCDR2 comprising the amino acid sequence of SEQ ID NO: 22,
(c) a HCDR3 comprising the amino acid sequence of SEQ ID NO: 23;
(d) a light chain complementarity determining region (LCDR1) comprising the amino acid sequence of SEQ ID NO: 24,
(e) a LCDR2 comprising the amino acid sequence of SEQ ID NO: 25, and
(f) a LCDR3 comprising the amino acid sequence of SEQ ID NO: 26, wherein:
the Xb residue in SEQ ID NO: 21 is V or I,
the Xc residue in SEQ ID NO: 21 is G or S,
the Xd residue in SEQ ID NO: 22 is T or R,
the Xg residue in SEQ ID NO: 23 is F or W,
the Xh residue in SEQ ID NO: 23 is R or Y,
the X1 residue in SEQ ID NO: 24 is D, G, A, or L,
the X2 residue in SEQ ID NO: 24 is S or A,
the X4 residue in SEQ ID NO: 25 is N or G,
the X5 residue in SEQ ID NO: 25 is N or K,
the X6 residue in SEQ ID NO: 26 is N or A,
the X7 residue in SEQ ID NO: 26 is V, D, or A,
the X8 residue in SEQ ID NO: 26 is S or Y, and
the X9 residue in SEQ ID NO: 26 is G or S,
and wherein the antibody or antigen-binding fragment thereof selectively binds to and activates TrkA and has
(i) a dissociation constant (K_{D}) for binding human TrkA ranging from about 0.1 nM to about 1,000 nM, preferably about 0.1 nM to about 10 nM, as determined by surface plasmon resonance; and/or
(ii) a half-maximal effective concentration (EC₅₀) value for binding human TrkA ranging from about 10 nM to about 1,000 nM, preferably about 100 nM to about 500 nM.

In an embodiment, in the anti-TrkA antibodies, or antigen-binding fragments thereof:
the Xb residue in SEQ ID NO: 21 is I,
the Xc residue in SEQ ID NO: 21 is S,
the Xd residue in SEQ ID NO: 22 is R,
the Xg residue in SEQ ID NO: 23 is W,
the Xh residue in SEQ ID NO: 23 is Y,
the X1 residue in SEQ ID NO: 24 is D,
the X2 residue in SEQ ID NO: 24 is A,
the X4 residue in SEQ ID NO: 25 is G,
the X5 residue in SEQ ID NO: 25 is N,
the X6 residue in SEQ ID NO: 26 is A,
the X7 residue in SEQ ID NO: 26 is A,
the X8 residue in SEQ ID NO: 26 is S, and
the X9 residue in SEQ ID NO: 26 is S.

In an embodiment, in the anti-TrkA antibodies, or antigen-binding fragments thereof:
the Xb residue in SEQ ID NO: 21 is V,
the Xc residue in SEQ ID NO: 21 is G,
the Xd residue in SEQ ID NO: 22 is T,
the Xg residue in SEQ ID NO: 23 is F,
the Xh residue in SEQ ID NO: 23 is Y,
the X1 residue in SEQ ID NO: 24 is D,
the X2 residue in SEQ ID NO: 24 is S,
the X4 residue in SEQ ID NO: 25 is N,
the X5 residue in SEQ ID NO: 25 is N,
the X6 residue in SEQ ID NO: 26 is N,
the X7 residue in SEQ ID NO: 26 is V,
the X8 residue in SEQ ID NO: 26 is S, and
the X9 residue in SEQ ID NO: 26 is G.

In an embodiment, in the anti-TrkA antibodies, or antigen-binding fragments thereof:
the Xb residue in SEQ ID NO: 21 is I,
the Xc residue in SEQ ID NO: 21 is S,
the Xd residue in SEQ ID NO: 22 is R,
the Xg residue in SEQ ID NO: 23 is W,
the Xh residue in SEQ ID NO: 23 is Y,
the X1 residue in SEQ ID NO: 24 is D,
the X2 residue in SEQ ID NO: 24 is S,
the X4 residue in SEQ ID NO: 25 is N,
the X5 residue in SEQ ID NO: 25 is N,
the X6 residue in SEQ ID NO: 26 is N,
the X7 residue in SEQ ID NO: 26 is D,
the X8 residue in SEQ ID NO: 26 is S, and
the X9 residue in SEQ ID NO: 26 is S.

In an embodiment, TrkA activation is measured by one or more of:
neurite outgrowth,
dimerization of TrkA,
internalization of TrkA,
phosphorylation of TrkA, or
TrkA signaling (including for example, activation of an intracellular pathways associated with neurite outgrowth and neurite survival, including MAPK pathway and PI3K pathways (e.g. as measured by assessing Erk and/or Akt phosphorylation)).

Disclosed herein is an anti-TrkA antibody, or antigen-binding fragment thereof, that cross-competes for binding to tropomyosin receptor kinase A (TrkA) with a reference antibody which comprises:
(a) a HCDR1 comprising the amino acid sequence of SEQ ID NO: 21,
(b) a HCDR2 comprising the amino acid sequence of SEQ ID NO: 22,
(c) a HCDR3 comprising the amino acid sequence of SEQ ID NO: 23;
(d) a LCDR1 comprising the amino acid sequence of SEQ ID NO: 24,
(e) a LCDR2 comprising the amino acid sequence of SEQ ID NO: 25, and
(f) a LCDR3 comprising the amino acid sequence of SEQ ID NO: 26.

Also disclosed are pharmaceutical compositions comprising the anti-TrkA antibodies or antigen-binding fragments thereof.

Also disclosed are nucleic acid molecules encoding the anti-TrkA antibodies or antigen-binding fragments thereof, vectors comprising the nucleic acid molecules, and cells transformed to express the antibody molecules.

Also disclosed herein are hybridomas prepared from transgenic animals, wherein the hybridomas produce the disclosed anti-TrkA antibodies or antigen-binding fragments thereof.

Also disclosed herein are uses of any of the herein disclosed anti-TrkA antibodies or antigen-binding fragments thereof, for treating disorders in which neuronal dysfunction is associated with the disease pathology, preferably wherein the anti-TrkA antibodies bind to TrkA and activate TrkA thereby treating the pathologies.

Also disclosed herein are uses of any of the herein disclosed anti-TrkA antibodies or antigen-binding fragments thereof for promoting neurite regeneration in a subject in need thereof.

Also disclosed herein are methods of treating disorders in which neuronal dysfunction is associated with the disease pathology, comprising administering a therapeutically effective amount of any of the herein disclosed anti-TrkA antibodies or antigen-binding fragments thereof to the subject to thereby treat the pathology.

Also disclosed herein are methods of promoting neurite regeneration in a subject in need thereof.

Also disclosed are uses of any of the herein disclosed anti-TrkA antibodies or antigen-binding fragments thereof in the manufacture of a medicament for treating disorders in which neuronal dysfunction is associated with the disease pathology, preferably wherein the anti-TrkA antibodies or antigen-binding fragments thereof bind to TrkA and activate TrkA and/or one or more downstream pathways thereby treating the pathologies.

Also disclosed are uses of any of the herein disclosed anti-TrkA antibodies or antigen-binding fragments thereof in the manufacture of a medicament for promoting neurite regeneration in a subject in need thereof.

In embodiments, anti-TrkA antibodies or antigen-binding fragments thereof as disclosed herein are monoclonal antibodies, preferably human.

Other embodiments of the disclosed antibodies and uses will be evident from the following detailed description of.

### BRIEF DESCRIPTION OF THE DRAWINGS

The summary, as well as the following detailed description, is further understood when read in conjunction with the appended drawings. For the purpose of illustrating the disclosed antibodies or antigen-binding fragments thereof, methods, and uses, there are shown in the drawings exemplary embodiments of the human antibodies or antigen-binding fragments thereof, methods, and uses; however, the antibodies or antigen-binding fragments thereof, methods, and uses are not limited to the specific embodiments disclosed.

FIG. 1 shows neurite outgrowth in pheochromocytoma (PC12) cells for several anti-TrkA antibodies in the absence of NGF following staining of neurites with β III tubulin. NGF 50 ng/ml was used as a positive control. One way ANOVA followed by Dunnett's multiple comparisons test against the isotype control antibody (140 nM). Data are mean±SEM. * p<0.05, ** p<0.01, 888p<0.001, **** p<0.0001.

FIG. 2 shows the results of pErk signaling assays for several IgG2 and IgG4 anti-TrkA antibodies. PC12 cells were stimulated for 15 minutes with various concentrations of anti-TrkA antibodies (140 nM-8.5 pM) (Ab3, Ab386, Ab449, Ab476, Ab523 and Ab676) having an IgG2 or IgG4 constant region in the absence of NGF. 2 nM mouse β NGF was used as a positive control and phosphorylated Erk signal measured. pEC₅₀ values were taken as the negative log EC₅₀ calculated from the concentration response using the nonlinear fit curve with variable slope four parameter equation in GraphPad Prism (n=2). Data are mean±SEM.

FIG. 3 shows the results of pAkt signaling assays for several IgG2 and IgG4 anti-TrkA variants of antibodies. PC12 cells were stimulated for 15 minutes with various concentrations of the anti-TrkA antibodies (140 nM-8.5 pM) (Ab3, Ab386, Ab449, Ab476, Ab523 and Ab676) having an IgG2 or IgG4 constant region in the absence of NGF. 2 nM mouse β NGF was used as a positive control and phosphorylated Akt signal measured. pEC₅₀ values were taken as the negative log EC₅₀ calculated from the concentration response using the nonlinear fit curve with variable slope four parameter equation in GraphPad Prism (n=2). Data are mean±SEM.

FIG. 4 shows neurite outgrowth in PC12 cells assessed at 138 hours post-treatment with murine βNGF or full length, F(ab′)2 or Fab versions of anti-TrkA antibody Ab82 or an isotype control. Data are mean±SEM; n=5.

FIG. 5A, FIG. 5B, and FIG. 5C show the neurotrophic capabilities of anti-TrkA antibody, Ab82, in PC12 cells. Panel (FIG. 5A) shows total branches, (FIG. 5B) shows total outgrowth and (FIG. 5C) shows total processes measured after Ab82 treatment at various concentrations on NGF-differentiated PC12 cells after NGF withdrawal. Data are mean±SEM; n=5. Statistical analysis: Two-way ANOVA; **** p<0.0001.

FIG. 6 shows axonal regrowth as total immunoreactive area (as pixels) in primary adult DRG sensory neurons 48 hours after axotomy and treatment with either isotype control or anti-TrkA antibody, Ab82, in the axonal compartment. Data are presented as bar graphs with mean±SEM; n=3. Statistical analysis: One-way ANOVA followed by Dunnett's multiple comparisons test against the isotype control antibody (30 nM); ** p<0.001. One-way ANOVA followed by Dunnett's multiple comparisons test against the vehicle control; #p<0.05, ##p<0.001.

FIG. 7 shows binding of anti-TrkA antibody, Ab82, to SH-SY5Y cells by flow cytometry. An increase in median fluorescence intensity (MFI) is a measurement of positive staining. Data are mean±SEM.

FIG. 8A, FIG. 8B, and FIG. 8C show that the anti-TrkA antibody Ab82 does not bind to other members of the protein superfamily related to TrkA, namely (FIG. 8A) human TrkB (hTrkB), (FIG. 8B) human TrkC (hTrkC), and (FIG. 8C) human p75^{NTR} (hp75^{NTR}) by surface plasmon resonance. For positive control, neurotrophin 4 (NT4), neurotrophin 3 (NT3), and brain-derived neurotrophic factor (BDNF), natural ligands to TrkB, TrkC, and p75^{NTR}, respectively, were used.

FIG. 9 shows concentration response curves of TrkA signaling (normalized to total Akt levels) in PC12 cells for full length anti-TrkA antibody Ab82, Ab82 F(ab′)2, Ab82 Fab, isotype control antibodies and β-NGF. Curves are depicted as an average of three independent experiments. Data are mean±SEM.

FIG. 10 shows concentration response curves of pErk signaling (normalized to total Erk levels) in PC12 cells for full length Ab82, Ab82 F(ab′)2, Ab82 Fab, isotype control antibodies and β-NGF. Curves are depicted as an average of three independent experiments. Data are mean±SEM.

FIG. 11 shows concentration response curves of pAkt signaling (normalized to total Akt levels) in PC12 cells for full length Ab82, Ab82 F(ab′)2, Ab82 Fab, isotype control antibodies and β-NGF. Curves are depicted as an average of three independent experiments. Data are mean±SEM.

FIG. 12 shows kinetic quantification of antibody internalization in HEK293T cells transiently expressing human, cynomolgus monkey or mouse TrkA. Antibody internalization was measured in live cells by labelling antibodies with Zenon pHrodo Red, a pH sensitive reagent that fluoresces in acidic compartments.

FIG. 13 shows neuronal support capabilities in the superior cervical ganglion (SCG) growth model in neonatal mice administered various doses (0.8 μg/kg-5 mg/kg) of anti-TrkA antibody Ab82 in comparison to isotype control antibody (5 mg/kg). Data are presented as bar graphs with mean±SEM; n=9. Statistical analysis: One-way ANOVA followed by Bonferroni's multiple comparisons test against the isotype control antibody; * p<0.05 and *** p<0.001. One-way ANOVA followed by Bonferroni's multiple comparisons test against the vehicle (PBS); ##p<0.01.

FIG. 14A, FIG. 14B, FIG. 14C, FIG. 14D, FIG. 14E, and FIG. 14F shows images of PGP9.5 (pan-neuronal marker) immunostaining of paw skin collected at week 11 from (FIG. 14A) naïve wildtype (WT) mice or db/db diabetic mice treated with (FIG. 14B) vehicle (saline), (FIG. 14C) isotype control antibody (5 mg/kg), (FIG. 14D) anti-TrkA antibody Ab82 (0.5 mg/kg) or (FIG. 14E) anti-TrkA antibody Ab82 (5 mg/kg) used to quantify intra-epidermal nerve fiber density (IENFD). White arrows indicate green PGP9.5 positive cutaneous innervation crossing from the epidermis to dermis. (FIG. 14F) IENFD quantification in paw skin. Data are presented as bar graphs with mean±SEM; n=10. Statistical analysis: One-way ANOVA followed by Dunnett's multiple comparisons test against the vehicle (saline); #p<0.05 and ###p<0.001. One-way ANOVA followed by Dunnett's multiple comparisons test against the isotype control antibody; *** p<0.001.

FIG. 15, FIG. 16, FIG. 17, FIG. 18, FIG. 19, and FIG. 20 show sequence alignments of several variants of anti-TrkA antibodies disclosed herein. FIGS. 15-17 show heavy chain sequences comprising HCDR1-3, respectively. FIGS. 18-20 show light chain sequences comprising LCDR1-3, respectively. Shaded residues denote several substitutions that were introduced to the variants of the anti-TrkA antibodies.

FIG. 21A and FIG. 21B show the restoration of neurite length in ND7/23 cells following glucose exposure by treatment with Ab82 or NGF. Cells were treated with Ab82 (ranging from 0.1 to 10 nM) or NGF (0.05 nM) following a transient (24 hr) glucose insult. Panel FIG. 21A is a graph of neurite outgrowth over the entire assay duration. Data is presented as mean±SEM, N=5, two-Tailed unpaired t-test. Panel FIG. 21B is a graph showing EC₅₀ as calculated from a concentration response using the nonlinear fit curve with variable slope three parameter equation in GraphPad Prism, for 60 hr time point (n=5).

FIG. 22A and FIG. 22B show the restoration of neurite branch points in ND7/23 cells following glucose exposure by treatment with Ab82 or NGF. Cells were treated with Ab82 (ranging from 0.1 to 10 nM) or NGF (0.05 nM) following a transient (24 hr) glucose insult. Panel FIG. 22A is a graph of neurite branching over the entire assay duration. Data is presented as mean±SEM, N=5, two-Tailed unpaired t-test. Panel FIG. 22B is a graph showing EC₅₀ as calculated from a concentration response using the nonlinear fit curve with variable slope three parameter equation in GraphPad Prism, for 60 hr time point (n=5).

FIG. 23A, FIG. 23B, FIG. 23C, FIG. 23D, FIG. 23E, and FIG. 23F show the protection of ND7/23 cells from neuronal damage upon glucose insult following the treatment with Ab82 or NGF. Panel FIG. 23A shows representative images taken at the 84 hr time point using the ImageXpress High Content Screen (HCS; ×20 magnification), where ND7/23 cells were live stained with Propidium iodide (PI), Calcein-AM Green and Hoechst (nuclear staining). Panels FIG. 23B is a graph showing the percentage of cells positive to PI following 84 hrs treatment and panel FIG. 23C is a corresponding graph showing IC50 as calculated from a concentration response using the nonlinear fit curve with variable slope three parameter equation in GraphPad Prism, for 84 hr time point (n=5)). Image analysis was performed using the MetaXpress HCS analysis program. Panels FIG. 23D to FIG. 23F are graphs which represent the use of calcein staining to assess cell viability (% positive cells, panel FIG. 23D), cell area (panel FIG. 23E) and membrane permeability (fluorescence intensity, panel FIG. 23F) Data is presented as mean±SEM, N=5, Two-Tailed unpaired t-test, ** p<0.01, *** p<0.001, *p<0.0001.

### DETAILED DESCRIPTION

### Definitions

The terms “a,” “an,” “the” and similar referents used in the context of describing the disclosed antibodies and uses (especially in the context of the following claims) are to be construed to cover both the singular and the plural, unless otherwise indicated herein or clearly contradicted by context.

The terms “comprise”, “comprises”, “comprised” or “comprising”, “including” or “having” and the like in the present specification and claims are used in an inclusive sense, that is to specify the presence of the stated features but not preclude the presence of additional or further features.

Specific embodiments disclosed herein may be further limited in the claims using “consisting of” or “consisting essentially of” language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s).

Embodiments of the antibodies and uses so claimed are inherently or expressly disclosed and enabled herein.

All methods disclosed herein can be performed in any suitable order unless otherwise indicated herein or otherwise clearly contradicted by context.

The use of any and all examples, or exemplary language (e.g. “such as”) disclosed herein is intended merely to better illuminate the antibodies and uses and does not pose a limitation on the scope of the antibodies and uses otherwise claimed. No language in the specification should be construed as indicating any non-claimed element essential to the practice of the antibodies and uses.

Except where otherwise indicated, all numbers expressing quantities of ingredients, reaction conditions, and so forth used in the specification and claims are to be understood as being modified in all instances by the term ‘about’. Accordingly, unless indicated to the contrary, the numerical parameters of the following specification and attached claims are approximations that may vary depending upon the desired properties sought to be obtained by the disclosed antibodies and uses. At the very least, each numerical parameter should be construed in light of the number of significant digits and ordinary rounding conventions. The term “about” may be understood to refer to a range of ±10%, such as ±5% or ±1% or, ±0.1%.

Recitation of ranges of values herein is merely intended to serve as a shorthand method of referring individually to each separate value falling within the range. Unless otherwise indicated herein, each individual value is incorporated into the specification as if it were individually recited herein. For example, if a range is from about 1 to about 50, it is deemed to include, for example, 1, 7, 34, 46.1, 23.7, or any other value or range within the range.

As used herein, “treat”, “treating” or “treatment” of a disease or disorder means that at least one or more symptoms improves, even if not all necessarily do. For example, these terms refer to utilizing an approach for obtaining beneficial or desired clinical results, including but not limited to an approach that achieves such beneficial or desired clinical results, wherein clinical results can include therapeutic measures that improve, cure, slow down, lessen symptoms of, and/or halt progression of a pathologic condition or disorder. Those in need of treatment can include those already diagnosed with or suspected of having the disorder. The terms “treat”, “treating” or “treatment” do not necessarily imply that a subject is treated until total recovery. The terms “treatment” and “treat” also refer to the maintenance and/or promotion of health in an individual not suffering from a disease but who may be susceptible to the development of an unhealthy condition. The terms “treatment” and “treat” are also intended to include the potentiation or otherwise enhancement of one or more primary prophylactic or therapeutic measures. As non-limiting examples, a treatment can be performed by a patient, a caregiver, a doctor, a nurse, or another healthcare professional.

As used herein, “prevent”, “preventing”, “prevention”, or “prophylaxis” of a disease or disorder means preventing that a disorder occurs in subject. “Prevention”, “preventing”, “prevention”, or “prophylaxis” includes reduction of risk, incidence and/or severity of a condition or disorder.

As used herein, the expressions “is for administration” and “is to be administered” have the same meaning as “is prepared to be administered”. In other words, the statement that an active compound “is for administration” has to be understood in that said active compound (e.g. an anti-TrkA antibody according to the present disclosure) has been formulated and made up into doses so that said active compound is in a state capable of exerting its therapeutic activity.

The terms “therapeutically effective amount” or “therapeutic amount” are intended to mean that amount of the disclosed anti-TrkA antibodies or antigen-binding fragments thereof alone or in combination with other therapeutic agents, that will provide a therapeutic benefit in the treatment or management of the biological or medical response of a tissue, a system, animal or human that is being sought by a researcher, veterinarian, medical doctor or other clinician. For example, the term “therapeutically effective amount” refers to an amount of the disclosed anti-TrkA antibodies or pharmaceutical compositions comprising the same, as disclosed herein, effective to achieve a particular biological or therapeutic or prophylactic result such as, but not limited to, biological or therapeutic results disclosed, disclosed, or exemplified herein. The therapeutically effective amount may vary according to factors such as the disease state, age, sex, and weight of the individual, and the ability of the composition to cause a desired response in a subject. The exact amount of a composition including a “therapeutically effective amount” will depend on the purpose of the treatment and will be ascertainable by one skilled in the art using known techniques.

The term “pharmaceutically acceptable” as used herein refers to substances that do not cause substantial adverse allergic or immunological reactions when administered to a subject. A “pharmaceutically acceptable carrier” or “pharmaceutical acceptable excipient” includes, but is not limited to, solvents, coatings, dispersion agents, wetting agents, isotonic and absorption delaying agents and disintegrants. Further, “pharmaceutically acceptable carrier” or “pharmaceutical acceptable excipient” includes any material which, when combined with an active ingredient (such as the disclosed antibodies or antigen-binding fragments thereof), allows the ingredient to retain biological activity and is non-reactive with the subject's immune system. Compositions comprising such carriers are formulated by well-known conventional methods (see, for example, Remington's Pharmaceutical Sciences, 18th edition, A. Gennaro, ed., Mack Publishing Co., Easton, Pa., 1990; and Remington, The Science and Practice of Pharmacy 20th Ed. Mack Publishing, 2000).

As used herein, “administer”, “administration”, “administering”, “administering to the subject” and similar terms refer to the delivery of the anti-TrkA antibodies or antigen-binding fragments thereof by an administration route including, but not limited to, intravenous, intra-arterial, intracranial, intramuscular, intraperitoneal, subcutaneous, intramuscular, or combinations thereof. The term includes, but is not limited to, administration by a medical professional and self-administration.

The term “subject” as used herein is intended to mean monkeys, such as cynomolgus macaques, and humans, and most preferably humans. “Subject” and “patient” are used interchangeably.

The term “antibody”, and like terms, is used in a broad sense and includes an immunoglobulin molecule that recognizes and binds to the TrkA receptor through at least one antigen binding domain within the variable region of the immunoglobulin molecule. An antibody of this disclosure encompasses full length antibodies (including for example, full length monoclonal antibodies), or antigen-binding fragments thereof (such as Fab, Fab′, F(ab′)2, and Fv fragments), single chain Fv (scFv) mutants, multispecific antibodies such as bispecific antibodies generated from at least two full length antibodies, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity.

An antibody can be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively.

The antibodies of the present disclosure can include one or more variable regions. A variable region of an antibody refers to the heavy chain variable region (HCVR) or the light chain variable region (LCVR), either alone or in combination. The variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain (e.g. in the sequence FR1, CDR1, FR2, CDR2, FR3, CDR3, FR4 (from amino to carboxy termini), contribute to the formation of the antigen-binding site of antibodies. The three CDRs in the HCVR are identified as HCDR 1, 2, and 3, and the three CDRs in the LCVR are identified as LCDR 1, 2, and 3 respectively. The location and size of the CDRs are defined based on rules which identify regions of sequence variability within the immunoglobulin variable regions. The Kabat numbering system is generally used when referring to a residue in the variable region (approximately residues 1-107 of the light chain and residues 1-113 of the heavy chain). According to the Kabat system, a heavy chain variable region can include a single amino acid insert (e.g. residue 52a according to Kabat) after residue 52 of H2 and inserted residues (e.g. residues 82a, 82b, and 82c, etc) after heavy chain FR residue 82. The Kabat numbering of residues can be determined for a given antibody by alignment at regions of homology of the sequence of the antibody with a “standard” Kabat numbered sequence. Chothia refers instead to the location of the structural loops. The end of the Chothia VH-CDR1 loop when numbered using the Kabat numbering convention varies at the VH between positions 32 and 34 depending on the length of the loop (this is because the Kabat numbering scheme places the insertions at VH in position 35A and 35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops and can be found in Oxford Molecular's AbM antibody modelling software. For all antibodies disclosed herein, the constant and/or variable region numbering can also be according to the IMGT (the international ImMunoGeneTics information System). The IMGT numbering scheme was originally based on alignment of germ-line V genes, spanning from FR1 to the beginning of the CDR3. The scheme was later extended to cover the entire variable region. The numbering runs from 1 to 128 based on the V-gene sequence alignment, with an insertion point only between positions 111 and 112 in the CDR3 for lengths exceeding 13 amino acids.

A full-length antibody can include a four-polypeptide unit consisting of two identical heavy chains and two identical light chains, held together by disulfide bonds. The light chains are generally shorter, with lower molecular weights than the heavy chains. Each polypeptide chain has a constant region and a variable region. The variable region is specific to each particular antibody. The light chain variable region is referred to as VL and the light chain constant region as CL. Similarly, the heavy chain variable region is referred to as VH and the heavy chain constant regions as CH, with CH1, CH2, and CH3 each denoting a different portion of the constant region of the heavy chain. In some embodiments, carbohydrates can be normally attached to the CH2 domains of the heavy chains. Further, a full-length antibody can also contain a fragment crystallizable (Fc) region. The Fc region contains only constant regions from the heavy chains (CH2 and CH3). In contrast, the fragment antigen-binding region (Fab) can include both a constant domain and the variable domains of both the heavy and light chains (VH, VL, CH1 and CL). A fragment variable region (Fv) contains only the two variable domains.

As disclosed above, the antibodies of the present disclosure can include one or more constant regions. A “constant region” of an antibody is a well-known term in the art and refers to the part of the antibody that is relatively constant in amino acid sequence between different molecules. Typically, the heavy chain constant region is composed of three distinct regions, termed CH1, CH2, and CH3, numbered in the direction from the amino terminal (N-terminal) end to the carboxy terminal (C-terminal) end. A typical light chain only has one constant region, termed CL. The constant region of an antibody determines its particular effector function. One of skill in the art will readily understand the terminology and structural features of constant regions of antibodies.

The term “Human antibody” refers to an antibody having heavy and light chain variable regions in which both the framework and the antigen binding sites are derived from sequences of human origin. A human antibody comprises heavy or light chain variable regions that are derived from sequences of human origin if the variable regions of the antibody are obtained from a system that uses human germline immunoglobulin or rearranged human immunoglobulin genes. Such systems include human immunoglobulin gene libraries displayed on phage, and transgenic non-human animals such as mice or rats carrying human immunoglobulin loci. A “Human antibody” may contain amino acid differences when compared to the human germline or rearranged immunoglobulin sequences due to, for example, naturally occurring somatic mutations or intentional introduction of substitutions in the framework or antigen binding sites. Typically, a “human antibody” is at least about 80%, 81%, 82%, 83%, 84%, 85%, 86%, 87%, 88%, 89%, 90%, 91%, 92%, 93%, 94%, 95%, 96%, 97%, 98%, 99%, or 100% identical in amino acid sequence to an amino acid sequence encoded by a human germline or rearranged immunoglobulin gene. Human antibodies, while derived from human immunoglobulin sequences, may be generated using systems such as phage display incorporating synthetic CDRs and/or synthetic frameworks, and/or can be subjected to in vitro mutagenesis to improve antibody properties in the variable regions or the constant regions or both, resulting in antibodies that do not naturally exist within the human antibody germline repertoire in vivo. If an antibody contains a constant region, the constant region also is derived from sequences of human origin. The heavy chain and light chain constant region sequences of some exemplary antibodies are disclosed below in Table 1 and Table 2.

TABLE 1
Heavy chain constant region sequences

| ID | CH Sequence |
|---|---|
| Human IgG1 | ASTKGPSVFPLAPSSKSTSGGTAALGCLVKDYFPEPVTVSWNSGALTSGVH |
| (SEQ ID NO: 1) | TFPAVLQSSGLYSLSSVVTVPSSSLGTQTYICNVNHKPSNTKVDKKVEPKSC |
| | DKTHTCPPCPAPELLGGPSVFLFPPKPKDTLMISRTPEVTCVVVDVSHEDPE |
| | VKFNWYVDGVEVHNAKTKPREEQYNSTYRVVSVLTVLHQDWLNGKEYKCK |
| | VSNKALPAPIEKTISKAKGQPREPQVYTLPPSRDELTKNQVSLTCLVKGFYPS |
| | DIAVEWESNGQPENNYKTTPPVLDSDGSFFLYSKLTVDKSRWQQGNVFSCS |
| | VMHEALHNHYTQKSLSLSPG |
| Human IgG1 | ASTKGPSVFPLAPSSKSTSGGTAALGCLVKDYFPEPVTVSWNSGALTSGVH |
| FAB TAG | TFPAVLQSSGLYSLSSVVTVPSSSLGTQTYICNVNHKPSNTKVDKKVEPKSC |
| (SEQ ID NO: 2) | |
| Human IgG1 | ASTKGPSVFPLAPSSKSTSGGTAALGCLVKDYFPEPVTVSWNSGALTSGVH |
| KiH Hole | TFPAVLQSSGLYSLSSVVTVPSSSLGTQTYICNVNHKPSNTKVDKKVEPKSC |
| (SEQ ID NO: 3) | DKTHTCPPCPAPELLGGPSVFLFPPKPKDTLMISRTPEVTCVVVDVSHEDPE |
| | VKFNWYVDGVEVHNAKTKPREEQYNSTYRVVSVLTVLHQDWLNGKEYKCK |
| | VSNKALPAPIEKTISKAKGQPREPQVYTLPPSRDELTKNQVSLSCAVKGFYPS |
| | DIAVEWESNGQPENNYKTTPPVLDSDGSFFLVSKLTVDKSRWQQGNVFSCS |
| | VMHEALHNHYTQKSLSLSPG |
| Human IgG1 | ASTKGPSVFPLAPSSKSTSGGTAALGCLVKDYFPEPVTVSWNSGALTSGVH |
| KIH Knob | TFPAVLQSSGLYSLSSVVTVPSSSLGTQTYICNVNHKPSNTKVDKKVEPKSC |
| (SEQ ID NO: 4) | DKTHTCPPCPAPELLGGPSVFLFPPKPKDTLMISRTPEVTCVVVDVSHEDPE |
| | VKFNWYVDGVEVHNAKTKPREEQYNSTYRVVSVLTVLHQDWLNGKEYKCK |
| | VSNKALPAPIEKTISKAKGQPREPQVYTLPPSRDELTKNQVSLWCLVKGFYP |
| | SDIAVEWESNGQPENNYKTTPPVLDSDGSFFLYSKLTVDKSRWQQGNVFSC |
| | SVMHEALHNHYTQKSLSLSPG |
| Human IgG1 | ASTKGPSVFPLAPSSKSTSGGTAALGCLVKDYFPEPVTVSWNSGALTSGVH |
| (L235A, G237A) | TFPAVLQSSGLYSLSSVVTVPSSSLGTQTYICNVNHKPSNTKVDKKVEPKSC |
| (SEQ ID NO: 5) | DKTHTCPPCPAPELAGAPSVFLFPPKPKDTLMISRTPEVTCVVVDVSHEDPE |
| | VKFNWYVDGVEVHNAKTKPREEQYNSTYRVVSVLTVLHQDWLNGKEYKCK |
| | VSNKALPAPIEKTISKAKGQPREPQVYTLPPSRDELTKNQVSLTCLVKGFYPS |
| | DIAVEWESNGQPENNYKTTPPVLDSDGSFFLYSKLTVDKSRWQQGNVFSCS |
| | VMHEALHNHYTQKSLSLSPG |
| Human IgG1 | ASTKGPSVFPLAPSSKSTSGGTAALGCLVKDYFPEPVTVSWNSGALTSGVH |
| YTE | TFPAVLQSSGLYSLSSVVTVPSSSLGTQTYICNVNHKPSNTKVDKKVEPKSC |
| (SEQ ID NO: 6) | DKTHTCPPCPAPELLGGPSVFLFPPKPKDTLYITREPEVTCWVDVSHEDPEV |
| | KFNWYVDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGFYPSD |
| | IAVEWESNGQPENNYKTTPPVLDSDGSFFLYSKLTVDKSRWQQGNVFSCSV |
| | MHEALHNHYTQKSLSLSPG |
| Human IgG1- | ASTKGPSVFPLAPSSKSTSGGTAALGCLVKDYFPEPVTVSWNSGALTSGVH |
| LALA-PG | TFPAVLQSSGLYSLSSVVTVPSSSLGTQTYICNVNHKPSNTKVDKKVEPKSC |
| (L234A/L235A/ | DKTHTCPPCPAPESTRGPSVFLFPPKPKDTLMISRTPEVTCVVVDVSHEDPE |
| P329G) | VKFNWYVDGVEVHNAKTKPREEQYNSTYRVVSVLTVLHQDWLNGKEYKCK |
| (SEQ ID NO: 7) | VSNKALPAPIEKTISKAKGQPREPQVYTLPPSRDELTKNQVSLTCLVKGFYPS |
| | DIAVEWESNGQPENNYKTTPPVLDSDGSFFLYSKLTVDKSRWQQGNVFSCS |
| | VMHEALHNHYTQKSLSLSPG |
| Human IgG1- | ASTKGPSVFPLAPSSKSTSGGTAALGCLVKDYFPEPVTVSWNSGALTSGVH |
| STR | TFPAVLQSSGLYSLSSVVTVPSSSLGTQTYICNVNHKPSNTKVDKKVEPKSC |
| (L234S/L235T/ | DKTHTCPPCPAPEAAGGPSVFLFPPKPKDTLMISRTPEVTCVVVDVSHEDPE |
| G236R) | VKFNWYVDGVEVHNAKTKPREEQYNSTYRVVSVLTVLHQDWLNGKEYKCK |
| (SEQ ID NO: 8) | VSNKALGAPIEKTISKAKGQPREPQVYTLPPSRDELTKNQVSLTCLVKGFYPS |
| | DIAVEWESNGQPENNYKTTPPVLDSDGSFFLYSKLTVDKSRWQQGNVFSCS |
| | VMHEALHNHYTQKSLSLSPG |
| Human | ASTKGPSVFPLAPCSRSTSESTAALGCLVKDYFPEPVTVSWNSGALTSGVHT |
| IgG2DASS | FPAVLQSSGLYSLSSVVTVPSSNFGTQTYTCNVDHKPSNTKVDKTVERKCCV |
| (SEQ ID NO: 9) | ECPPCPAPPVAGPSVFLFPPKPKDTLMISRTPEVTCVVVDVSHEDPEVQFNW |
| | YVDGVEVHNAKTKPREEQFNSTFRVVSVLTVVHQDWLNGKEYKCKVSNKGL |
| | PSSIEKTISKTKGQPREPQVYTLPPSREEMTKNQVSLTCLVKGFYPSDIAVEW |
| | ESNGQPENNYKTTPPMLDSDGSFFLYSKLTVDKSRWQQGNVFSCSVMHEA |
| | LHNHYTQKSLSLSPG |
| Human IgG4 | ASTKGPSVFPLAPCSRSTSESTAALGCLVKDYFPEPVTVSWNSGALTSGVHT |
| (SEQ ID NO: | FPAVLQSSGLYSLSSVVTVPSSSLGTKTYTCNVDHKPSNTKVDKRVESKYGP |
| 10) | PCPPCPAPEFLGGPSVFLFPPKPKDTLMISRTPEVTCVVVDVSQEDPEVQFN |
| | WYVDGVEVHNAKTKPREEQFNSTYRVVSVLTVLHQDWLNGKEYKCKVSNK |
| | GLPSSIEKTISKAKGQPREPQVYTLPPSQEEMTKNQVSLTCLVKGFYPSDIAV |
| | EWESNGQPENNYKTTPPVLDSDGSFFLYSRLTVDKSRWQEGNVFSCSVMH |
| | EALHNHYTQKSLSLSLG |
| Human IgG4 | STKGPSVFPLAPCSRSTSESTAALGCLVKDYFPEPVTVSWNSGALTSGVHTF |
| KiH Hole | PAVLQSSGLYSLSSVVTVPSSSLGTKTYTCNVDHKPSNTKVDKRVESKYGPP |
| (SEQ ID NO: | CPPCPAPEFLGGPSVFLFPPKPKDTLMISRTPEVTCVVVDVSQEDPEVQFNW |
| 11) | YVDGVEVHNAKTKPREEQFNSTYRVVSVLTVLHQDWLNGKEYKCKVSNKGL |
| | PSSIEKTISKAKGQPREPQVYTLPPSQEEMTKNQVSLWCLVKGFYPSDIAVE |
| | WESNGQPENNYKTTPPVLDSDGSFFLYSRLTVDKSRWQEGNVFSCSVMHE |
| | ALHNHYTQKSLSLSLG |
| Human IgG4 | ASTKGPSVFPLAPCSRSTSESTAALGCLVKDYFPEPVTVSWNSGALTSGVHT |
| KIH Knob | FPAVLQSSGLYSLSSVVTVPSSSLGTKTYTCNVDHKPSNTKVDKRVESKYGP |
| (SEQ ID NO: | PCPPCPAPEFLGGPSVFLFPPKPKDTLMISRTPEVTCVWVDVSQEDPEVQFN |
| 12) | WYVDGVEVHNAKTKPREEQFNSTYRVVSVLTVLHQDWLNGKEYKCKVSNK |
| | GLPSSIEKTISKAKGQPREPQVYTLPPSQEEMTKNQVSLWCLVKGFYPSDIA |
| | VEWESNGQPENNYKTTPPVLDSDGSFFLYSRLTVDKSRWQEGNVFSCSVM |
| | HEALHNHYTQKSLSLSLG |
| Human IgG4 | ASTKGPSVFPLAPCSRSTSESTAALGCLVKDYFPEPVTVSWNSGALTSGVHT |
| (L235A, G237A) | FPAVLQSSGLYSLSSVVTVPSSSLGTKTYTCNVDHKPSNTKVDKRVESKYGP |
| (SEQ ID NO: | PCPPCPAPEFAGAPSVFLFPPKPKDTLMISRTPEVTCVVVDVSQEDPEVQFN |
| 13) | WYVDGVEVHNAKTKPREEQFNSTYRVVSVLTVLHQDWLNGKEYKCKVSNK |
| | GLPSSIEKTISKAKGQPREPQVYTLPPSQEEMTKNQVSLTCLVKGFYPSDIAV |
| | EWESNGQPENNYKTTPPVLDSDGSFFLYSRLTVDKSRWQEGNVFSCSVMH |
| | EALHNHYTQKSLSLSLG |
| Human IgG4 | ASTKGPSVFPLAPCSRSTSESTAALGCLVKDYFPEPVTVSWNSGALTSGVHT |
| (L235E) | FPAVLQSSGLYSLSSVVTVPSSSLGTKTYTCNVDHKPSNTKVDKRVESKYGP |
| (SEQ ID NO: | PCPPCPAPEFEGGPSVFLFPPKPKDTLMISRTPEVTCVVVDVSQEDPEVQFN |
| 14) | WYVDGVEVHNAKTKPREEQFNSTYRVVSVLTVLHQDWLNGKEYKCKVSNK |
| | GLPSSIEKTISKAKGQPREPQVYTLPPSQEEMTKNQVSLTCLVKGFYPSDIAV |
| | EWESNGQPENNYKTTPPVLDSDGSFFLYSRLTVDKSRWQEGNVFSCSVMH |
| | EALHNHYTQKSLSLSLG |
| Human IgG4 | ASTKGPSVFPLAPCSRSTSESTAALGCLVKDYFPEPVTVSWNSGALTSGVHT |
| YTE | FPAVLQSSGLYSLSSVVTVPSSSLGTKTYTCNVDHKPSNTKVDKRVESKYGP |
| (SEQ ID NO: | PCPPCPAPEFLGGPSVFLFPPKPKDTLYITREPEVTCVVVDVSQEDPEVQFN |
| 15) | WYVDGVEVHNAKTKPREEQFNSTYRVVSVLTVLHQDWLNGKEYKCKVSNK |
| | GLPSSIEKTISKAKGQPREPQVYTLPPSQEEMTKNQVSLTCLVKGFYPSDIAV |
| | EWESNGQPENNYKTTPPVLDSDGSFFLYSRLTVDKSRWQEGNVFSCSVMH |
| | EALHNHYTQKSLSLSLG |
| Human IgG4 | ASTKGPSVFPLAPCSRSTSESTAALGCLVKDYFPEPVTVSWNSGALTSGVHT |
| YTE KIH Hole | FPAVLQSSGLYSLSSVVTVPSSSLGTKTYTCNVDHKPSNTKVDKRVESKYGP |
| (SEQ ID NO: | PCPPCPAPEFLGGPSVFLFPPKPKDTLYITREPEVTCVVVDVSQEDPEVQFN |
| 16) | WYVDGVEVHNAKTKPREEQFNSTYRVVSVLTVLHQDWLNGKEYKCKVSNK |
| | GLPSSIEKTISKAKGQPREPQVYTLPPSQEEMTKNQVSLSCAVKGFYPSDIAV |
| | EWESNGQPENNYKTTPPVLDSDGSFFLVSRLTVDKSRWQEGNVFSCSVMH |
| | EALHNHYTQKSLSLSLG |
| Human IgG4 | ASTKGPSVFPLAPCSRSTSESTAALGCLVKDYFPEPVTVSWNSGALTSGVHT |
| YTE KIH Knob | FPAVLQSSGLYSLSSVVTVPSSSLGTKTYTCNVDHKPSNTKVDKRVESKYGP |
| (SEQ ID NO: | PCPPCPAPEFLGGPSVFLFPPKPKDTLYITREPEVTCVVVDVSQEDPEVQFN |
| 17) | WYVDGVEVHNAKTKPREEQFNSTYRVVSVLTVLHQDWLNGKEYKCKVSNK |
| | GLPSSIEKTISKAKGQPREPQVYTLPPSQEEMTKNQVSLWCLVKGFYPSDIA |
| | VEWESNGQPENNYKTTPPVLDSDGSFFLYSRLTVDKSRWQEGNVFSCSVM |
| | HEALHNHYTQKSLSLSLG |
| Human IgG1 | ASTKGPSVFPLAPSSKSTSGGTAALGCLVKDYFPEPVTVSWNSGALTSGVH |
| (L235A, | TFPAVLQSSGLYSLSSVVTVPSSSLGTQTYICNVNHKPSNTKVDKKVEPKSC |
| G237A), YTE | DKTHTCPPCPAPELAGAPSVFLFPPKPKDTLYITREPEVTCVVVDVSHEDPEV |
| (SEQ ID NO: | KFNWYVDGVEVHNAKTKPREEQYNSTYRVVSVLTVLHQDWLNGKEYKCKV |
| 18) | SNKALPAPIEKTISKAKGQPREPQVYTLPPSRDELTKNQVSLTCLVKGFYPSD |
| | IAVEWESNGQPENNYKTTPPVLDSDGSFFLYSKLTVDKSRWQQGNVFSCSV |
| | MHEALHNHYTQKSLSLSPG |

Exemplary light constant region is disclosed in Table 2.

TABLE 2
Light chain constant region sequences

| ID | CL Sequence |
|---|---|
| Human kappa | RTVAAPSVFIFPPSDEQLKSGTASVVCLLNNFYPRE |
| (SEQ ID | AKVQWKVDNALQSGNSQESVTEQDSKDSTYSLSSTL |
| NO: 19) | TLSKADYEKHKVYACEVTHQGLSSPVTKSFNRGEC |
| Human lambda | GQPKAAPSVTLFPPSSEELQANKATLVCLISDFYPG |
| (SEQ ID | AVTVAWKADSSPVKAGVETTTPSKQSNNKYAASSYL |
| NO: 20) | SLTPEQWKSHRSYSCQVTHEGSTVEKTVAPTECS |

The heavy chain of the anti-TrkA antibodies of the present disclosure may comprise a constant region, such as a constant region that is disclosed in Table 1. In some embodiments, the constant region is modified such that it is immunologically inert, for example several modified constant region are disclosed in Table 1. For example, the residues to which modifications were introduce to, in order to provide the modified constant region are listed herein in Table 1, using the EU numbering base on IgG1 sequence (i.e., SEQ ID NO: 1).

The heavy chain constant region may be a glycosylated for N-linked glycosylation. In some embodiments, the constant region can be a glycosylated for N-linked glycosylation by mutating the oligosaccharide attachment residue (such as Asn297) and/or flanking residues that are part of the N-glycosylation recognition sequence in the constant region. In some embodiments, the constant region can be a glycosylated for N-linked glycosylation. The constant region can be a glycosylated for N-linked glycosylation enzymatically or by expression in a glycosylation deficient host cell.

An antibody disclosed herein can be a monoclonal antibody. A “monoclonal antibody” refers to a homogeneous antibody population involved in the highly specific recognition and binding of a single antigenic determinant, or epitope. The term “monoclonal antibody” encompasses both full length antibodies as well as antigen-binding fragments, mutants, fusion proteins comprising an antibody portion, and any other modified immunoglobulin molecule comprising an antigen recognition site that are capable of binding TrkA and activating TrkA. Furthermore, “monoclonal antibody” refers to such antibodies made in any number of manners including but not limited to by hybridoma, phage selection, recombinant expression, and transgenic animals.

The term “epitope” or “antigenic determinant” are used interchangeably herein and refer to that portion of an antigen capable of being recognized and specifically bound by a particular antibody. When the antigen is a polypeptide, epitopes can be formed both from contiguous amino acids and non-contiguous amino acids juxtaposed by tertiary folding of a protein. Epitopes formed from contiguous amino acids are typically retained upon protein denaturing, whereas epitopes formed by tertiary folding are typically lost upon protein denaturing.

“Variant” refers to a polypeptide or a polynucleotide that differs from a reference polypeptide or a reference polynucleotide by one or more modifications for example, substitutions, insertions, or deletions. In embodiments, the disclosed antibodies or antigen-binding fragments thereof can comprise one or more substitutions, deletions, or insertions, in the framework, constant and/or CDR regions. In embodiments, antibodies of the present disclosure are engineered to contain a heavy/light chain variable framework region that is the product of or derived from the human genes IGHV4-4*02 and IGLV3-19*01, respectively, and more specifically the IGHV4-4*02 heavy chain and IGLV3-19*01 light chain. Examples of variants that are derived from the above human germline can have a heavy chain and/or a light chain variable framework region that comprises at least 1 amino acid modification up to at least 5, 10, 15 or 20 modifications from the corresponding heavy chain and/or light chain variable framework region of a corresponding non-human antibody. Thus, the GL3 variant as disclosed herein is an example of a functional variant that is a product of or derived from the human genes IGHV4-4*02 heavy chain and IGLV3-19*01 light chain, respectively, as summarized below:

TABLE 3
Human Sequences

| ID | Sequence |
|---|---|
| IGHV4-4*02 | QVQLQESGPGLVKPSGTLSLTCAVSGGSISSSN |
| heavy chain | WWSWVRQPPGKGLEWIGEIYHSGSTNYNPSLKS |
| (SEQ ID NO: 67) | RVTISVDKSKNQFSLKLSSVTAADTAVYYCAR |
| IGLV3-19*01 | SSELTQDPAVSVALGQTVRITCQGDSLRSYYAS |
| light chain | WYQQKPGQAPVLVIYGKNNRPSGIPDRFSGSSS |
| (SEQ ID NO: 68) | GNTASLTITGAQAEDEADYYCNSRDSSGNH |
| GL3 variant | QVQLQESGPGLVKPSGTLSLTCAVSGGSISSSN |
| heavy chain | WWTWVRQPPGKGLEWIGEIYHRGNTNYNPSLKS |
| (SEQ ID NO: 69) | RVTISVDKSKNQFSLKLSSVTAADTAVYYCARA |
| | PFFDYWGQGTLV |
| GL3 variant | SSELTQDPAVSVALGQTVRITCQGDSLRSYYAS |
| light chain | WYQQKPGQAPVLVIYGKNNRPSGIPDRFSGSSS |
| (SEQ ID NO: 70) | GNTASLTITGAQAEDEADYYCNSQDSSGNHLIF |
| | GGGTKLTVLG |

The term “receptor activation”, “activation” or “activate”, when referring to a receptor (e.g. TrkA), unless otherwise specified, means that the receptor is in a state competent to promote TrkA biological response (e.g. neuron cell survival and differentiation) and/or induction of downstream pathway(s) mediated by TrkA signal transduction (i.e. signaling). For example, TrkA activation may be caused by an agent (e.g. an anti-TrkA antibody) capable of binding TrkA, resulting in TrkA dimerization, phosphorylation, and internalization to promote a biological response (e.g. neuron cell survival and differentiation) and/or induction of downstream pathway(s) mediated by TrkA signal transduction (i.e. signaling). Various assays for assessing activation of TrkA are known in the art. For example, TrkA activation may be measured by one or more of the following: neurite outgrowth assay, TrkA signaling assays (including for example, activation of an intracellular pathway including MAPK and PI3K pathways as measured by assessing for example, Erk and/or Akt phosphorylation), dimerization of TrkA, internalization of TrkA, or phosphorylation of TrkA. Included within the definition is activation which produces a submaximal biological response and/or submaximal induction of downstream pathway(s). Thus, receptor activation by a full agonist that produces a maximal (i.e. normal) biological response and/or induction of downstream pathway(s) in an assay may be distinguished from receptor activation by a partial agonist that produces a submaximal biological response (i.e. lower than normal) and/or submaximal induction of downstream pathway(s). The above disclosed submaximal or maximal biological response and/or submaximal or maximal induction of downstream pathway(s) can be measured in any of the disclosed assays in comparison to the endogenous ligand/agonist (i.e. NGF or any other bivalent receptor-binding agents that causes an equivalent biological response and/or an equivalent induction of downstream pathway(s) as the endogenous ligand/agonist). For example, maximal biological response and/or maximal induction of downstream pathway(s) can be achieved by a full agonist to provide a biological response (e.g. as measured by one or more of the above-disclosed assays), which is almost identical or completely identical to the biological response and/or to the induction of downstream pathway(s) that can be achieved by the NGF. Submaximal biological response and/or submaximal induction of downstream pathway(s) can be achieved by a partial agonist to provide a biological response (e.g. as measured for example by one or more of the above-disclosed assays), which is lower (i.e. at least about 45% to about 85% lower) than the response that can be achieved by NGF. Preferably, the above disclosed submaximal or maximal biological response may be determined in the in vitro neurite outgrowth assay (for example, as shown in Example 6). Preferably, the above disclosed submaximal or maximal induction of downstream pathway(s) may be determined in any TrkA signaling assays (including for example, activation of an intracellular pathway including MAPK and PI3K pathways as measured by assessing for example, Erk and/or Akt phosphorylation. For example, as shown in Example 3).

“Neurite outgrowth” refers to the complex process where extension of neurites allows neurons to accomplish precise connectivity and proper functioning of the nervous system. Neural crest-derived, rat pheochromocytoma cell line PC12 is a widely used model of the sympathetic and sensory nervous system that responds to nerve growth factor (NGF) and enables measurement of neurite outgrowth in vitro.

As used herein, the phrase “anti-TrkA antibodies or antigen-binding fragments thereof” refers to an antibody, or antigen-binding fragment thereof, that specifically binds to TrkA. The phrases “specifically binds” or “binds specifically to”, or variations thereof, refer to the ability of the disclosed antibodies or antigen-binding fragments thereof to preferentially bind to TrkA. Hence, antibody molecules that specifically bind TrkA are substantially free of other antibodies having different binding specificities. Antibody molecules that specifically bind human TrkA, however, can have cross-reactivity to other antigens, such as orthologs of human TrkA, such as mouse, rat, rabbit, porcine or monkey derived TrkA. The antibodies disclosed herein are able to specifically bind to both naturally produced human TrkA and to human TrkA which is recombinantly produced in mammalian or prokaryotic cells. For example, the antibodies disclosed herein are able to specifically bind human TrkA extracellular domain (e.g. hTrkA-ECD-His) as exemplified in examples 11 and 12 and does not bind to the extracellular domain (ECD) of human TrkB (hTrkB), human TrkC (hTrkC), and human p75^{NTR} (hp75^{NTR}).

“Binding affinity” generally refers to the strength of the sum total of noncovalent interactions between a single binding site of a molecule (e.g. an antibody) and its binding partner (e.g. an antigen/target). Unless indicated otherwise, as used herein, “binding affinity” refers to intrinsic binding affinity which reflects a 1:1 interaction between members of a binding pair (e.g. antibody and antigen). The affinity of a molecule X for its partner Y can generally be represented by the equilibrium dissociation constant (KD). Affinity can be measured by common methods known in the art, including those disclosed herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present disclosure, an example of which is an affinity ELISA assay. In addition, affinity can be determined by a surface plasmon resonance assay (SPR, e.g. BIAcore®-based assay). Using this methodology, the association rate constant (Ka in M−1s−1) and the dissociation rate constant (Kd in s−1) can be measured. The equilibrium dissociation constant (KD in M) can then be calculated from the ratio of the kinetic rate constants (Kd/Ka). Binding affinity can be also determined by a kinetic method, such as a Kinetic Exclusion Assay (KinExA) as disclosed in Rathanaswami et al. Analytical Biochemistry, Vol. 373:52-60, 2008. Using a KinExA assay, the equilibrium dissociation constant (KD in M) and the association rate constant (Ka in M−1s−1) 1) can be measured. The dissociation rate constant (Kd in s−1) can be calculated from these values KD×Ka). Binding affinity can be also determined by an equilibrium/solution method.

Further examples of the disclosed antibodies and uses are disclosed below. However, it should be noted that the disclosed antibodies and uses should not be limited to these examples, and that the disclosed antibodies and uses are susceptible to variations, modifications and/or additions other than those specifically disclosed, and it is to be understood that the disclosed antibodies and uses include all such variations, modifications and/or additions which fall within the scope of the claims.

Disclosed herein are antibodies or antigen-binding fragments thereof, which specifically bind to and activate TrkA. The inventors have surprisingly found that the anti-TrkA antibodies of the present disclosure bind to and activate TrkA, but to a lower extent than NGF activation of TrkA.

The anti-tropomyosin receptor kinase A (TrkA) antibodies, or an antigen-binding fragments thereof, can comprise:
a heavy chain complementarity determining region 1 (HCDR1) comprising the amino acid sequence of SEQ ID NO: 71, a HCDR2 comprising the amino acid sequence of SEQ ID NO: 72, a HCDR3 comprising the amino acid sequence of SEQ ID NO: 73, a light chain complementarity determining region 1 (LCDR1) comprising the amino acid sequence of SEQ ID NO: 74, a LCDR2 comprising the amino acid sequence of SEQ ID NO: 75, and a LCDR3 comprising the amino acid sequence of SEQ ID NO: 76,
wherein the HCDR1 is numbered according to AbM and the remaining CDRs are numbered according to Kabat.

The anti-TrkA antibodies, or an antigen-binding fragments thereof, can comprise a heavy chain variable region (VH) comprising the amino acid sequence of SEQ ID NO: 49 and a light chain variable region (VL) comprising the amino acid sequence of SEQ ID NO: 50. In some embodiments, the anti-TrkA antibodies, or antigen-binding fragments thereof, can comprise a heavy chain constant region comprising the amino acid sequence selected from the group consisting of: SEQ ID NO: 1; 2; 3; 4; 5; 6; 7; 8; 9; 10; 11; 12; 13; 14; 15; 16; 17 and 18. In some embodiments, the anti-TrkA antibody comprises a light chain which is a human kappa light chain. In some embodiments, the anti-TrkA antibody comprises a light chain which is a human lambda light chain. In some embodiments, the anti-TrkA antibodies, or antigen-binding fragments thereof, can comprise a light chain constant region comprising the amino acid sequence selected from the group consisting of: SEQ ID NO: 19 and SEQ ID NO: 20. In some embodiments, the anti-TrkA antibodies, or antigen-binding fragments thereof, can comprise:
(a) a heavy chain constant region of an amino acid sequence of SEQ ID NO: 1, 2, 3, 4, 5, 6, 7, 8, 9, 10, 11, 12, 13, 14, 15, 16, 17 or 18, and
(b) a light chain constant region of an amino acid sequence of SEQ ID NO: 19 or SEQ ID NO: 20.

In some embodiments, the anti-TrkA antibodies, or antigen-binding fragments thereof, can comprise a heavy chain constant region comprising the amino acid sequence of SEQ ID NO: 5 and a light chain constant region comprising the amino acid sequence of SEQ ID NO: 20.

In some embodiments, the anti-TrkA antibodies, or antigen-binding fragments thereof, are monoclonal antibodies.

In some embodiments, the anti-TrkA antibodies, or antigen-binding fragments thereof, are human antibodies. Thus, disclosed herein are human anti-TrkA antibodies, or antigen-binding fragments thereof, comprising:
a heavy chain complementarity determining region 1 (HCDR1) comprising the amino acid sequence of SEQ ID NO: 71, a HCDR2 comprising the amino acid sequence of SEQ ID NO: 72, a HCDR3 comprising the amino acid sequence of SEQ ID NO: 73, a light chain complementarity determining region 1 (LCDR1) comprising the amino acid sequence of SEQ ID NO: 74, a LCDR2 comprising the amino acid sequence of SEQ ID NO: 75, and a LCDR3 comprising the amino acid sequence of SEQ ID NO: 76
wherein the HCDR1 is numbered according to AbM and the remaining CDRs are numbered according to Kabat.

The antigen-binding fragment of the disclosed antibodies can be a F(ab′)2. In some embodiments, the F(ab′)2 comprises a heavy chain variable region and hinge region comprising the amino acid sequence of SEQ ID NO: 79 and a light chain variable region comprising the amino acid sequence of SEQ ID NO: 77.

In some embodiments, the antigen-binding fragment of the anti-TrkA antibodies is not a Fab fragment. Thus, disclosed herein are human anti-TrkA antigen-binding fragments, comprising:
a heavy chain complementarity determining region 1 (HCDR1) comprising the amino acid sequence of SEQ ID NO: 71, a HCDR2 comprising the amino acid sequence of SEQ ID NO: 72, a HCDR3 comprising the amino acid sequence of SEQ ID NO: 73, a light chain complementarity determining region 1 (LCDR1) comprising the amino acid sequence of SEQ ID NO: 74, a LCDR2 comprising the amino acid sequence of SEQ ID NO: 75, and a LCDR3 comprising the amino acid sequence of SEQ ID NO: 76,
wherein the HCDR1 is numbered according to AbM and the remaining CDRs are numbered according to Kabat and wherein the antibody-binding fragment is not a Fab fragment.

The anti-TrkA antibodies, or antigen-binding fragments thereof, can comprise an IgG1 heavy chain constant region, an IgG2 heavy chain constant region, or an IgG4 heavy chain constant region. In some embodiments, the anti-TrkA antibodies, or antigen-binding fragments thereof, comprise an IgG1 heavy chain constant region that comprises an L235A substitution and a G237A substitution. In some embodiments, anti-TrkA antibodies, or antigen-binding fragments thereof, can comprise a heavy chain (HC) constant region comprising the amino acid sequence of SEQ ID NO: 5 and a light chain (LC) constant region comprising the amino acid sequence of SEQ ID NO: 20.

The anti-TrkA antibodies, or antigen-binding fragments thereof, can comprise a heavy chain (HC) comprising the amino acid sequence of SEQ ID NO: 77 and a light chain (LC) comprising the amino acid sequence of SEQ ID NO: 78.

Also disclosed herein are anti-TrkA antibodies, or antigen-binding fragments thereof, comprising:
(a) a HCDR1 comprising the amino acid sequence of SEQ ID NO: 21,
(b) a HCDR2 comprising the amino acid sequence of SEQ ID NO: 22,
(c) a HCDR3 comprising the amino acid sequence of SEQ ID NO: 23;
(d) a LCDR1 comprising the amino acid sequence of SEQ ID NO:24,
(e) a LCDR2 comprising the amino acid sequence of SEQ ID NO: 25, and
(f) a LCDR3 comprising the amino acid sequence of SEQ ID NO: 26.

In some embodiments, the anti-TrkA antibodies, or antigen-binding fragments thereof, are human antibodies or antigen-binding fragments thereof comprising:
(a) a HCDR1 comprising the amino acid sequence of SEQ ID NO: 21,
(b) a HCDR2 comprising the amino acid sequence of SEQ ID NO: 22,
(c) a HCDR3 comprising the amino acid sequence of SEQ ID NO: 23;
(d) a LCDR1 comprising the amino acid sequence of SEQ ID NO:24,
(e) a LCDR2 comprising the amino acid sequence of SEQ ID NO:25, and
(f) a LCDR3 comprising the amino acid sequence of SEQ ID NO:26.

In some embodiments, the antigen-binding fragments comprise:
(a) a HCDR1 comprising the amino acid sequence of SEQ ID NO: 21,
(b) a HCDR2 comprising the amino acid sequence of SEQ ID NO: 22,
(c) a HCDR3 comprising the amino acid sequence of SEQ ID NO: 23;
(d) a LCDR1 comprising the amino acid sequence of SEQ ID NO:24,
(e) a LCDR2 comprising the amino acid sequence of SEQ ID NO:25, and
(f) a LCDR3 comprising the amino acid sequence of SEQ ID NO:26
wherein the antigen-binding fragment is not a Fab fragment.

In some embodiments, in the anti-TrkA antibodies or antigen-binding fragments thereof:
the Xb residue in SEQ ID NO: 21 is V or I,
the Xc residue in SEQ ID NO: 21 is G or S,
the Xd residue in SEQ ID NO: 22 is T or R,
the Xg residue in SEQ ID NO: 23 is F or W,
the Xh residue in SEQ ID NO: 23 is R or Y,
the X1 residue in SEQ ID NO: 24 is D, G, A or L,
the X2 residue in SEQ ID NO: 24 is S or A,
the X4 residue in SEQ ID NO: 25 is N or G,
the X5 residue in SEQ ID NO: 25 is N or K,
the X6 residue in SEQ ID NO: 26 is N or A,
the X7 residue in SEQ ID NO: 26 is V, D or A,
the X8 residue in SEQ ID NO: 26 is S or Y, and
the X9 residue in SEQ ID NO: 26 is G or S.

In an embodiment, in the anti-TrkA antibodies, or antigen-binding fragments thereof:
the Xb residue in SEQ ID NO: 21 is I,
the Xc residue in SEQ ID NO: 21 is S,
the Xd residue in SEQ ID NO: 22 is R,
the Xg residue in SEQ ID NO: 23 is W,
the Xh residue in SEQ ID NO: 23 is Y,
the X1 residue in SEQ ID NO: 24 is D,
the X2 residue in SEQ ID NO: 24 is A,
the X4 residue in SEQ ID NO: 25 is G,
the X5 residue in SEQ ID NO: 25 is N,
the X6 residue in SEQ ID NO: 26 is A,
the X7 residue in SEQ ID NO: 26 is A,
the X8 residue in SEQ ID NO: 26 is S, and
the X9 residue in SEQ ID NO: 26 is S.

In an embodiment, in the anti-TrkA antibodies, or antigen-binding fragments thereof:
the Xb residue in SEQ ID NO: 21 is V,
the Xc residue in SEQ ID NO: 21 is G,
the Xd residue in SEQ ID NO: 22 is T,
the Xg residue in SEQ ID NO: 23 is F,
the Xh residue in SEQ ID NO: 23 is Y,
the X1 residue in SEQ ID NO: 24 is D,
the X2 residue in SEQ ID NO: 24 is S,
the X4 residue in SEQ ID NO: 25 is N,
the X5 residue in SEQ ID NO: 25 is N,
the X6 residue in SEQ ID NO: 26 is N,
the X7 residue in SEQ ID NO: 26 is V,
the X8 residue in SEQ ID NO: 26 is S, and
the X9 residue in SEQ ID NO: 26 is G.

In an embodiment, in the anti-TrkA antibodies, or antigen-binding fragments thereof:
the Xb residue in SEQ ID NO: 21 is I,
the Xc residue in SEQ ID NO: 21 is S,
the Xd residue in SEQ ID NO: 22 is R,
the Xg residue in SEQ ID NO: 23 is W,
the Xh residue in SEQ ID NO: 23 is Y,
the X1 residue in SEQ ID NO: 24 is D,
the X2 residue in SEQ ID NO: 24 is S,
the X4 residue in SEQ ID NO: 25 is N,
the X5 residue in SEQ ID NO: 25 is N,
the X6 residue in SEQ ID NO: 26 is N,
the X7 residue in SEQ ID NO: 26 is D,
the X8 residue in SEQ ID NO: 26 is S, and
the X9 residue in SEQ ID NO: 26 is S.

Thus, disclosed herein are anti-TrkA antibodies, or antigen-binding fragments thereof, wherein the antibodies or antigen-binding fragments thereof comprise:
(a) a heavy chain complementarity determining region (HCDR1) comprising the amino acid sequence of SEQ ID NO: 21,
(b) a HCDR2 comprising the amino acid sequence of SEQ ID NO: 22,
(c) a HCDR3 comprising the amino acid sequence of SEQ ID NO: 23;
(d) a light chain complementarity determining region (LCDR1) comprising the amino acid sequence of SEQ ID NO: 24,
(e) a LCDR2 comprising the amino acid sequence of SEQ ID NO: 25, and
(f) a LCDR3 comprising the amino acid sequence of SEQ ID NO: 26,
wherein:
the Xb residue in SEQ ID NO: 21 is V or I,
the Xc residue in SEQ ID NO: 21 is G or S,
the Xd residue in SEQ ID NO: 22 is T or R,
the Xg residue in SEQ ID NO: 23 is F or W,
the Xh residue in SEQ ID NO: 23 is R or Y,
the X1 residue in SEQ ID NO: 24 is D, G, A, or L,
the X2 residue in SEQ ID NO: 24 is S or A,
the X4 residue in SEQ ID NO: 25 is N or G,
the X5 residue in SEQ ID NO: 25 is N or K,
the X6 residue in SEQ ID NO: 26 is N or A,
the X7 residue in SEQ ID NO: 26 is V, D, or A,
the X8 residue in SEQ ID NO: 26 is S or Y, and
the X9 residue in SEQ ID NO: 26 is G or S.

In an embodiment, in the anti-TrkA antibodies, or antigen-binding fragments thereof:
the Xb residue in SEQ ID NO: 21 is I,
the Xc residue in SEQ ID NO: 21 is S,
the Xd residue in SEQ ID NO: 22 is R,
the Xg residue in SEQ ID NO: 23 is W,
the Xh residue in SEQ ID NO: 23 is Y,
the X1 residue in SEQ ID NO: 24 is D,
the X2 residue in SEQ ID NO: 24 is A,
the X4 residue in SEQ ID NO: 25 is G,
the X5 residue in SEQ ID NO: 25 is N,
the X6 residue in SEQ ID NO: 26 is A,
the X7 residue in SEQ ID NO: 26 is A,
the X8 residue in SEQ ID NO: 26 is S, and
the X9 residue in SEQ ID NO: 26 is S.

In an embodiment, in the anti-TrkA antibodies, or antigen-binding fragments thereof:
the Xb residue in SEQ ID NO: 21 is V,
the Xc residue in SEQ ID NO: 21 is G,
the Xd residue in SEQ ID NO: 22 is T,
the Xg residue in SEQ ID NO: 23 is F,
the Xh residue in SEQ ID NO: 23 is Y,
the X1 residue in SEQ ID NO: 24 is D,
the X2 residue in SEQ ID NO: 24 is S,
the X4 residue in SEQ ID NO: 25 is N,
the X5 residue in SEQ ID NO: 25 is N,
the X6 residue in SEQ ID NO: 26 is N,
the X7 residue in SEQ ID NO: 26 is V,
the X8 residue in SEQ ID NO: 26 is S, and
the X9 residue in SEQ ID NO: 26 is G.

In an embodiment, in the anti-TrkA antibodies, or antigen-binding fragments thereof:
the Xb residue in SEQ ID NO: 21 is I,
the Xc residue in SEQ ID NO: 21 is S,
the Xd residue in SEQ ID NO: 22 is R,
the Xg residue in SEQ ID NO: 23 is W,
the Xh residue in SEQ ID NO: 23 is Y,
the X1 residue in SEQ ID NO: 24 is D,
the X2 residue in SEQ ID NO: 24 is S,
the X4 residue in SEQ ID NO: 25 is N,
the X5 residue in SEQ ID NO: 25 is N,
the X6 residue in SEQ ID NO: 26 is N,
the X7 residue in SEQ ID NO: 26 is D,
the X8 residue in SEQ ID NO: 26 is S, and
the X9 residue in SEQ ID NO: 26 is S.

In some embodiments, the antigen binding fragment is a F(ab′)2. In some embodiments, the F(ab′)2 comprises a heavy chain variable region and hinge region comprising the amino acid sequence of SEQ ID NO: 79. In some embodiments, the F(ab′)2 comprises light chain variable region comprising the amino acid sequence of SEQ ID NO: 77. In some embodiments, the F(ab′)2 comprises a heavy chain variable region and hinge region comprising the amino acid sequence of SEQ ID NO: 79 and a light chain variable region comprising the amino acid sequence of SEQ ID NO: 77.

In some embodiments, the anti-TrkA antibodies, or antigen-binding fragments thereof, are human antibodies or antigen-binding fragments thereof comprise:
(a) a heavy chain complementarity determining region (HCDR1) comprising the amino acid sequence of SEQ ID NO: 21,
(b) a HCDR2 comprising the amino acid sequence of SEQ ID NO: 22,
(c) a HCDR3 comprising the amino acid sequence of SEQ ID NO: 23;
(d) a light chain complementarity determining region (LCDR1) comprising the amino acid sequence of SEQ ID NO: 24,
(e) a LCDR2 comprising the amino acid sequence of SEQ ID NO: 25, and
(f) a LCDR3 comprising the amino acid sequence of SEQ ID NO: 26,
wherein:
the Xb residue in SEQ ID NO: 21 is V or I,
the Xc residue in SEQ ID NO: 21 is G or S,
the Xd residue in SEQ ID NO: 22 is T or R,
the Xg residue in SEQ ID NO: 23 is F or W,
the Xh residue in SEQ ID NO: 23 is R or Y,
the X1 residue in SEQ ID NO: 24 is D, G, A, or L,
the X2 residue in SEQ ID NO: 24 is S or A,
the X4 residue in SEQ ID NO: 25 is N or G,
the X5 residue in SEQ ID NO: 25 is N or K,
the X6 residue in SEQ ID NO: 26 is N or A,
the X7 residue in SEQ ID NO: 26 is V, D, or A,
the X8 residue in SEQ ID NO: 26 is S or Y, and
the X9 residue in SEQ ID NO: 26 is G or S.

In an embodiment, the human antibodies or antigen-binding fragments thereof comprise:
the Xb residue in SEQ ID NO: 21 is I,
the Xc residue in SEQ ID NO: 21 is S,
the Xd residue in SEQ ID NO: 22 is R,
the Xg residue in SEQ ID NO: 23 is W,
the Xh residue in SEQ ID NO: 23 is Y,
the X1 residue in SEQ ID NO: 24 is D,
the X2 residue in SEQ ID NO: 24 is A,
the X4 residue in SEQ ID NO: 25 is G,
the X5 residue in SEQ ID NO: 25 is N,
the X6 residue in SEQ ID NO: 26 is A,
the X7 residue in SEQ ID NO: 26 is A,
the X8 residue in SEQ ID NO: 26 is S, and
the X9 residue in SEQ ID NO: 26 is S.

In an embodiment, the human antibodies or antigen-binding fragments thereof comprise:
the Xb residue in SEQ ID NO: 21 is V,
the Xc residue in SEQ ID NO: 21 is G,
the Xd residue in SEQ ID NO: 22 is T,
the Xg residue in SEQ ID NO: 23 is F,
the Xh residue in SEQ ID NO: 23 is Y,
the X1 residue in SEQ ID NO: 24 is D,
the X2 residue in SEQ ID NO: 24 is S,
the X4 residue in SEQ ID NO: 25 is N,
the X5 residue in SEQ ID NO: 25 is N,
the X6 residue in SEQ ID NO: 26 is N,
the X7 residue in SEQ ID NO: 26 is V,
the X8 residue in SEQ ID NO: 26 is S, and
the X9 residue in SEQ ID NO: 26 is G.

In an embodiment, the human antibodies or antigen-binding fragments thereof comprise:
the Xb residue in SEQ ID NO: 21 is I,
the Xc residue in SEQ ID NO: 21 is S,
the Xd residue in SEQ ID NO: 22 is R,
the Xg residue in SEQ ID NO: 23 is W,
the Xh residue in SEQ ID NO: 23 is Y,
the X1 residue in SEQ ID NO: 24 is D,
the X2 residue in SEQ ID NO: 24 is S,
the X4 residue in SEQ ID NO: 25 is N,
the X5 residue in SEQ ID NO: 25 is N,
the X6 residue in SEQ ID NO: 26 is N,
the X7 residue in SEQ ID NO: 26 is D,
the X8 residue in SEQ ID NO: 26 is S, and
the X9 residue in SEQ ID NO: 26 is S.

The human antibodies or antigen-binding fragments thereof, can comprise a heavy chain variable region (VH) comprising the amino acid sequence of SEQ ID NO: 49 and a light chain variable region (VL) comprising the amino acid sequence of SEQ ID NO: 50. In some embodiments, the anti-TrkA antibodies, or antigen-binding fragments thereof, can comprise a heavy chain constant region comprising the amino acid sequence selected from the group consisting of: SEQ ID NO: 1; 2; 3; 4; 5; 6; 7; 8; 9; 10; 11; 12; 13; 14; 15; 16; 17 and 18. In some embodiments, the anti-TrkA antibody comprises a light chain which is a human kappa light chain. In some embodiments, the anti-TrkA antibody comprises a light chain which is a human lambda light chain. In some embodiments, the anti-TrkA antibodies, or antigen-binding fragments thereof, can comprise a light chain constant region comprising the amino acid sequence selected from the group consisting of: SEQ ID NO: 19 and SEQ ID NO: 20. In some embodiments, the anti-TrkA antibodies, or antigen-binding fragments thereof, can comprise:
(a) a heavy chain constant region of an amino acid sequence of SEQ ID NO: 1, 2, 3, 4, 5, 6, 7, 8, 9, 10, 11, 12, 13, 14, 15, 16, 17 or 18, and
(b) a light chain constant region of an amino acid sequence of SEQ ID NO: 19 or SEQ ID NO: 20.

In some embodiments, the anti-TrkA antibodies, or antigen-binding fragments thereof, can comprise a heavy chain constant region comprising the amino acid sequence of SEQ ID NO: 5 and a light chain constant region comprising the amino acid sequence of SEQ ID NO: 20.

In some embodiments, the antigen binding fragment is a F(ab′)2. In some embodiments, the F(ab′)2 comprises a heavy chain variable region and hinge region comprising the amino acid sequence of SEQ ID NO: 79. In some embodiments, the F(ab′)2 comprises light chain variable region comprising the amino acid sequence of SEQ ID NO: 77. In some embodiments, the F(ab′)2 comprises a heavy chain variable region and hinge region comprising the amino acid sequence of SEQ ID NO: 79 and a light chain variable region comprising the amino acid sequence of SEQ ID NO: 77.

In some embodiments, the antigen-binding fragment of the anti-TrkA antibodies is not a Fab fragment. Thus, disclosed herein are antigen-binding fragments comprising:
(a) a heavy chain complementarity determining region (HCDR1) comprising the amino acid sequence of SEQ ID NO: 21,
(b) a HCDR2 comprising the amino acid sequence of SEQ ID NO: 22,
(c) a HCDR3 comprising the amino acid sequence of SEQ ID NO: 23;
(d) a light chain complementarity determining region (LCDR1) comprising the amino acid sequence of SEQ ID NO: 24,
(e) a LCDR2 comprising the amino acid sequence of SEQ ID NO: 25, and
(f) a LCDR3 comprising the amino acid sequence of SEQ ID NO: 26,
wherein:
the Xb residue in SEQ ID NO: 21 is V or I,
the Xc residue in SEQ ID NO: 21 is G or S,
the Xd residue in SEQ ID NO: 22 is T or R,
the Xg residue in SEQ ID NO: 23 is F or W,
the Xh residue in SEQ ID NO: 23 is R or Y,
the X1 residue in SEQ ID NO: 24 is D, G, A, or L,
the X2 residue in SEQ ID NO: 24 is S or A,
the X4 residue in SEQ ID NO: 25 is N or G,
the X5 residue in SEQ ID NO: 25 is N or K,
the X6 residue in SEQ ID NO: 26 is N or A,
the X7 residue in SEQ ID NO: 26 is V, D, or A,
the X8 residue in SEQ ID NO: 26 is S or Y, and
the X9 residue in SEQ ID NO: 26 is G or S
wherein the antigen-binding fragment is not a Fab fragment.

In some embodiments, in the anti-TrkA antibodies or antigen-binding fragments thereof:
the Xb residue in SEQ ID NO: 21 is I,
the Xc residue in SEQ ID NO: 21 is S,
the Xd residue in SEQ ID NO: 22 is R,
the Xg residue in SEQ ID NO: 23 is W,
the Xh residue in SEQ ID NO: 23 is Y,
the X1 residue in SEQ ID NO: 24 is D,
the X2 residue in SEQ ID NO: 24 is A,
the X4 residue in SEQ ID NO: 25 is G,
the X5 residue in SEQ ID NO: 25 is N,
the X6 residue in SEQ ID NO: 26 is A,
the X7 residue in SEQ ID NO: 26 is A,
the X8 residue in SEQ ID NO: 26 is S, and
the X9 residue in SEQ ID NO: 26 is S.

In an embodiment, in the anti-TrkA antibodies, or antigen-binding fragments thereof:
the Xb residue in SEQ ID NO: 21 is V,
the Xc residue in SEQ ID NO: 21 is G,
the Xd residue in SEQ ID NO: 22 is T,
the Xg residue in SEQ ID NO: 23 is F,
the Xh residue in SEQ ID NO: 23 is Y,
the X1 residue in SEQ ID NO: 24 is D,
the X2 residue in SEQ ID NO: 24 is S,
the X4 residue in SEQ ID NO: 25 is N,
the X5 residue in SEQ ID NO: 25 is N,
the X6 residue in SEQ ID NO: 26 is N,
the X7 residue in SEQ ID NO: 26 is V,
the X8 residue in SEQ ID NO: 26 is S, and
the X9 residue in SEQ ID NO: 26 is G.

In an embodiment, in the anti-TrkA antibodies, or antigen-binding fragments thereof:
the Xb residue in SEQ ID NO: 21 is I,
the Xc residue in SEQ ID NO: 21 is S,
the Xd residue in SEQ ID NO: 22 is R,
the Xg residue in SEQ ID NO: 23 is W,
the Xh residue in SEQ ID NO: 23 is Y,
the X1 residue in SEQ ID NO: 24 is D,
the X2 residue in SEQ ID NO: 24 is S,
the X4 residue in SEQ ID NO: 25 is N,
the X5 residue in SEQ ID NO: 25 is N,
the X6 residue in SEQ ID NO: 26 is N,
the X7 residue in SEQ ID NO: 26 is D,
the X8 residue in SEQ ID NO: 26 is S, and
the X9 residue in SEQ ID NO: 26 is S.

Also disclosed herein are anti-TrkA antibodies or antigen-binding fragments thereof comprising:
(a) a heavy chain variable region (HCVR or VH) CDR1, CDR2 and CDR3 amino acid sequences derived from an amino acid sequence of SEQ ID NO:27, 29, 33, 35, 37, 39, 41, 43, 45, 47, 49, 51, 53, 57, 59, 61, 63 or 65, and
(b) a light chain variable region (LCVR or VL) CDR1, CDR2 and CDR3 amino acid sequences derived from an amino acid sequence of SEQ ID NO: 28, 30, 32, 34, 36, 38, 40, 42, 44, 46, 48, 50, 52, 54, 56, 58, 60, 62, 64 or 66,
wherein the CDR sequences are identified by the Kabat, Chothia, AbM or IMGT numbering schemes, or combinations thereof.

The anti-TrkA antibodies, or an antigen-binding fragments thereof, can comprise a heavy chain variable region (VH) comprising the amino acid sequence of SEQ ID NO: 49 and a light chain variable region (VL) comprising the amino acid sequence of SEQ ID NO: 50. In some embodiments, the anti-TrkA antibodies, or antigen-binding fragments thereof, can comprise a heavy chain constant region comprising the amino acid sequence selected from the group consisting of: SEQ ID NO: 1; 2; 3; 4; 5; 6; 7; 8; 9; 10; 11; 12; 13; 14; 15; 16; 17 and 18. In some embodiments, the anti-TrkA antibody comprises a light chain which is a human kappa light chain. In some embodiments, the anti-TrkA antibody comprises a light chain which is a human lambda light chain. In some embodiments, the anti-TrkA antibodies, or antigen-binding fragments thereof, can comprise a light chain constant region comprising the amino acid sequence selected from the group consisting of: SEQ ID NO: 19 and SEQ ID NO: 20. In some embodiments, the anti-TrkA antibodies, or antigen-binding fragments thereof, can comprise:
(a) a heavy chain constant region of an amino acid sequence of SEQ ID NO: 1, 2, 3, 4, 5, 6, 7, 8, 9, 10, 11, 12, 13, 14, 15, 16, 17 or 18, and
(b) a light chain constant region of an amino acid sequence of SEQ ID NO: 19 or SEQ ID NO: 20.

In some embodiments, the anti-TrkA antibodies, or antigen-binding fragments thereof, can comprise a heavy chain constant region comprising the amino acid sequence of SEQ ID NO: 5 and a light chain constant region comprising the amino acid sequence of SEQ ID NO: 20.

In some embodiments, the anti-TrkA antibody, as disclosed herein, selectively binds to, and activates TrkA. Thus, disclosed herein are anti-TrkA antibodies, or antigen-binding fragments thereof, wherein the antibodies or antigen-binding fragments thereof comprise:
(a) a heavy chain complementarity determining region (HCDR1) comprising the amino acid sequence of SEQ ID NO: 21,
(b) a HCDR2 comprising the amino acid sequence of SEQ ID NO: 22,
(c) a HCDR3 comprising the amino acid sequence of SEQ ID NO: 23;
(d) a light chain complementarity determining region (LCDR1) comprising the amino acid sequence of SEQ ID NO: 24,
(e) a LCDR2 comprising the amino acid sequence of SEQ ID NO: 25, and
(f) a LCDR3 comprising the amino acid sequence of SEQ ID NO: 26,
wherein:
the Xb residue in SEQ ID NO: 21 is V or I,
the Xc residue in SEQ ID NO: 21 is G or S,
the Xd residue in SEQ ID NO: 22 is T or R,
the Xg residue in SEQ ID NO: 23 is F or W,
the Xh residue in SEQ ID NO: 23 is R or Y,
the X1 residue in SEQ ID NO: 24 is D, G, A, or L,
the X2 residue in SEQ ID NO: 24 is S or A,
the X4 residue in SEQ ID NO: 25 is N or G,
the X5 residue in SEQ ID NO: 25 is N or K,
the X6 residue in SEQ ID NO: 26 is N or A,
the X7 residue in SEQ ID NO: 26 is V, D, or A,
the X8 residue in SEQ ID NO: 26 is S or Y, and
the X9 residue in SEQ ID NO: 26 is G or S,
and wherein the antibody or antigen-binding fragment thereof selectively binds to and activates TrkA and has
(i) a dissociation constant (KD) for binding human TrkA ranging from about 0.1 nM to about 1,000 nM, preferably about 0.1 nM to about 10 nM, as determined by surface plasmon resonance; and/or
(ii) a half-maximal effective concentration (EC₅₀) value for binding human TrkA ranging from about 10 nM to about 1,000 nM, preferably about 100 nM to about 500 nM. The antibodies or antigen-binding fragment can be a human antibody or antigen-binding fragment. In some embodiments, the antigen-binding fragment is not a Fab.

In an embodiment, in the anti-TrkA antibodies, or antigen-binding fragments thereof:
the Xb residue in SEQ ID NO: 21 is I,
the Xc residue in SEQ ID NO: 21 is S,
the Xd residue in SEQ ID NO: 22 is R,
the Xg residue in SEQ ID NO: 23 is W,
the Xh residue in SEQ ID NO: 23 is Y,
the X1 residue in SEQ ID NO: 24 is D,
the X2 residue in SEQ ID NO: 24 is A,
the X4 residue in SEQ ID NO: 25 is G,
the X5 residue in SEQ ID NO: 25 is N,
the X6 residue in SEQ ID NO: 26 is A,
the X7 residue in SEQ ID NO: 26 is A,
the X8 residue in SEQ ID NO: 26 is S, and
the X9 residue in SEQ ID NO: 26 is S.

In an embodiment, in the anti-TrkA antibodies, or antigen-binding fragments thereof:
the Xb residue in SEQ ID NO: 21 is V,
the Xc residue in SEQ ID NO: 21 is G,
the Xd residue in SEQ ID NO: 22 is T,
the Xg residue in SEQ ID NO: 23 is F,
the Xh residue in SEQ ID NO: 23 is Y,
the X1 residue in SEQ ID NO: 24 is D,
the X2 residue in SEQ ID NO: 24 is S,
the X4 residue in SEQ ID NO: 25 is N,
the X5 residue in SEQ ID NO: 25 is N,
the X6 residue in SEQ ID NO: 26 is N,
the X7 residue in SEQ ID NO: 26 is V,
the X8 residue in SEQ ID NO: 26 is S, and
the X9 residue in SEQ ID NO: 26 is G.

In an embodiment, in the anti-TrkA antibodies, or antigen-binding fragments thereof:
the Xb residue in SEQ ID NO: 21 is I,
the Xc residue in SEQ ID NO: 21 is S,
the Xd residue in SEQ ID NO: 22 is R,
the Xg residue in SEQ ID NO: 23 is W,
the Xh residue in SEQ ID NO: 23 is Y,
the X1 residue in SEQ ID NO: 24 is D,
the X2 residue in SEQ ID NO: 24 is S,
the X4 residue in SEQ ID NO: 25 is N,
the X5 residue in SEQ ID NO: 25 is N,
the X6 residue in SEQ ID NO: 26 is N,
the X7 residue in SEQ ID NO: 26 is D,
the X8 residue in SEQ ID NO: 26 is S, and
the X9 residue in SEQ ID NO: 26 is S.

The anti-TrkA antibodies, or an antigen-binding fragments thereof, can comprise a heavy chain variable region (VH) comprising the amino acid sequence of SEQ ID NO: 49 and a light chain variable region (VL) comprising the amino acid sequence of SEQ ID NO: 50. In some embodiments, the anti-TrkA antibodies, or antigen-binding fragments thereof, can comprise a heavy chain constant region comprising the amino acid sequence selected from the group consisting of: SEQ ID NO: 1; 2; 3; 4; 5; 6; 7; 8; 9; 10; 11; 12; 13; 14; 15; 16; 17 and 18. In some embodiments, the anti-TrkA antibody comprises a light chain which is a human kappa light chain. In some embodiments, the anti-TrkA antibody comprises a light chain which is a human lambda light chain. In some embodiments, the anti-TrkA antibodies, or antigen-binding fragments thereof, can comprise a light chain constant region comprising the amino acid sequence selected from the group consisting of: SEQ ID NO: 19 and SEQ ID NO: 20. In some embodiments, the anti-TrkA antibodies, or antigen-binding fragments thereof, can comprise:
(a) a heavy chain constant region of an amino acid sequence of SEQ ID NO: 1, 2, 3, 4, 5, 6, 7, 8, 9, 10, 11, 12, 13, 14, 15, 16, 17 or 18, and
(b) a light chain constant region of an amino acid sequence of SEQ ID NO: 19 or SEQ ID NO: 20.

In some embodiments, the anti-TrkA antibodies, or antigen-binding fragments thereof, can comprise a heavy chain constant region comprising the amino acid sequence of SEQ ID NO: 5 and a light chain constant region comprising the amino acid sequence of SEQ ID NO: 20.

In some embodiments, the anti-TrkA antibodies, or antigen-binding fragments thereof, can comprise a heavy chain constant region comprising the amino acid sequence of SEQ ID NO: 5 and a light chain constant region comprising the amino acid sequence of SEQ ID NO: 20.

In some embodiments, the antigen binding fragment is a F(ab′)2. In some embodiments, the F(ab′)2 comprises a heavy chain variable region and hinge region comprising the amino acid sequence of SEQ ID NO: 79. In some embodiments, the F(ab′)2 comprises light chain variable region comprising the amino acid sequence of SEQ ID NO: 77. In some embodiments, the F(ab′)2 comprises a heavy chain variable region and hinge region comprising the amino acid sequence of SEQ ID NO: 79 and a light chain variable region comprising the amino acid sequence of SEQ ID NO: 77.

Preferably, TrkA receptor activation is determined by methodology including the measurement of:
neurite outgrowth,
dimerization of TrkA,
internalization of TrkA including determining cellular localization,
phosphorylation of TrkA, and/or
TrkA signaling (including for example, activation of an intracellular pathways associated with neurite outgrowth and neurite survival, including MAPK pathway and PI3K pathways (e.g. as measured by assessing Erk and/or Akt phosphorylation).

Exemplary functional properties of the disclosed antibodies and antigen-binding fragments thereof are disclosed in the herein disclosed Examples.

In some embodiments, the anti-TrkA antibodies or antigen-binding fragments thereof, as disclosed herein, induce neurite outgrowth in the absence of NGF.

In some embodiments, the anti-TrkA antibodies or antigen-binding fragments thereof, as disclosed herein, acts to induce TrkA activation in the absence of NGF, when compared to a control of NGF induced TrkA activation when measured in the absence of the antibody (NGF-Ab). The anti-TrkA antibodies or antigen-binding fragments thereof can also act to induce TrkA activation in the presence of NGF, wherein NGF is presence in an amount that enables the anti-TrkA antibody to bind TrkA and activate TrkA. For example, the anti-TrkA antibody, as disclosed herein, acts to induce TrkA activation to produce a submaximal biological response in comparison to a control of NGF induced TrkA activation when measured in the absence of the antibody (NGF-Ab). The submaximal biological response is preferably measured using any standard methodology known to a person skilled in the art, such as the one or more that are disclosed herein. Further preferably, when an in vitro neurite outgrowth assay is determinative of TrkA activation, the increase in TrkA activation induced by the anti-TrkA antibody is at least about 45%, about 50%, about 55%, about 60%, about 65%, about 70%, about 75%, or about 80% up to about 85%, or from about 45% to about 50%, from about 50% to about 55%, from about 55% to about 60%, from about 60% to about 65%, from about 65% to about 70%, from about 70% to about 75%, from about 75% to about 80%, or from about 80% to about 85%, when compared to TrkA activation by NGF-Ab, or where the increase in TrkA activation induced by the anti-TrkA antibody is at most about 45%, about 50%, about 55%, about 60%, about 65%, about 70%, about 75%, about 80%, or about 85%, when compared to TrkA activation by NGF-Ab.

In some embodiments, the anti-TrkA antibodies or antigen-binding fragments thereof have an EC₅₀ value from about 50 pM to about 100 pM, preferably from about 70 pM to about 90 pM as determined by an in vitro neurite outgrowth assay comprising PC12 cells (i.e., under standard testing conditions), when compared to a control of NGF absent antibody having an EC₅₀ value from about 1 pM to about 15 pM, preferably from about 5 pM to about 8 pM.

The anti-TrkA antibodies or antigen-binding fragments thereof can have an EC₅₀ value from about 0.05 nM to about 0.5 nM, preferably from about 0.15 nM to about 2.5 nM as determined by an in vitro neurite outgrowth assay comprising ND7/23 cells subjected to glucose insult, when compared to a control of NGF absent antibody having an EC₅₀ value from about 0.1 nM to about 1.0 nM, preferably from about 0.3 nM to about 0.6 nM.

The anti-TrkA antibodies or antigen-binding fragments thereof, as disclosed herein, can exhibit a low or substantially no immunogenicity (i.e. low or substantially no adverse reactogenicity) when administered to humans. Mitigating against immunogenicity can be achieved, for example, by engineering the antibodies or antigen-binding fragments thereof to have higher humanness score as well as by removing residues which are found to have with higher risk of immunogenicity by predictive in vitro techniques. As used herein, a “humanness score” refers to the percent sequence identify of the antibody to a human germline. As such, in an embodiment, the anti-TrkA antibodies or antigen-binding fragments thereof, as disclosed herein, is a human antibody or is modified to closely resemble a human antibody, preferably wherein the humanness score of the anti-TrkA antibodies is at least about 90% or about 95% up to about 98%.

In some embodiments, the anti-TrkA antibodies or antigen-binding fragments thereof, as disclosed herein, is of the isotype IgG1, IgG2, IgG3, IgG4, IgM, IgA1, IgA2, a secretory IgA, IgD, or IgE, preferably IgG1, IgG2 or IgG4. Preferably, the anti-TrkA antibody, as disclosed herein, is a full-length antibody or antigen-binding fragment thereof. Further preferably, the anti-TrkA antibodies or antigen-binding fragments thereof as disclosed herein (including a full-length antibody and antigen-binding fragment thereof) bind to and activate TrkA. For example, the anti-TrkA antibody of the present disclosure (including a full-length antibody and antigen-binding fragment thereof) function as a bivalent receptor-binding agent, to cause TrkA dimerization and leads to autophosphorylation and internalization of TrkA, thereby promoting TrkA biological response (e.g. neuron cell survival and differentiation) and/or induction of downstream pathway(s) mediated by TrkA signal transduction (i.e. signaling). In an embodiment, the binding portion thereof or antigen-binding fragment of the disclosed antibodies is a F(ab′)2 fragment (i.e. having two antigen-binding (Fab) regions linked by disulfide bonds of an antibody without Fc region, providing a divalent antibody fragment), preferably excluding a Fab fragment (i.e. having one Fab region of an antibody without Fc region, providing a monovalent antibody fragment), unless engineered to provide activation of TrkA. For example, a monovalent antibody fragment can be engineered to have two binding sites that correctly orient TrkA receptors for activation.

In some embodiments, the anti-TrkA antibodies, as disclosed herein, can comprise a constant region that is an IgG constant region, preferably an IgG1, IgG2 or IgG4 constant region. Further preferably, the constant region comprises:
(a) a heavy chain constant region amino acid sequence of one of SEQ ID NO: 1-18, and
(a) a light chain constant region amino acid sequence of SEQ ID NO: 19, 20.

The anti-TrkA antibodies, as disclosed herein, can comprise a constant region sequence selected from the group consisting of the human IgG1 sequence of SEQ ID NO: 1, the human IgG1 FAB TAG sequence of SEQ ID NO: 2, the human IgG1 KiH Hole sequence of SEQ ID NO: 3, the human IgG1 KiH Knob sequence of SEQ ID NO: 4, the human IgG1 (L235A,G237A) sequence of SEQ ID NO: 5, the human IgG1 YTE sequence of SEQ ID NO: 6, the human IgG1-LALA-PG (L234A/L235A/P329G) sequence of SEQ ID NO: 7, the human IgG1-STR (L234S/L235T/G236R) sequence of SEQ ID NO: 8, and the human IgG1 (L235A, G237A, YTE) sequence of SEQ ID NO: 18. Preferably the constant region is the human IgG1 (L235A,G237A) sequence of SEQ ID NO: 5.

The anti-TrkA antibodies, as disclosed herein, can comprise a constant region that is a IgG2 constant region. Preferably, the constant region comprises the human IgG2DASS sequence of SEQ ID NO: 9.

The anti-TrkA antibodies, as disclosed herein, can comprise a constant region that is an IgG4 constant region. Preferably, the constant region comprises a sequence selected from the group consisting of the human IgG4 sequence of SEQ ID NO: 10, the human IgG4 KiH Hole sequence of SEQ ID NO: 11, the human IgG4 KiH Knob sequence of SEQ ID NO: 12, the human IgG4 (L235A,G237A) sequence of SEQ ID NO: 13, the human IgG4 (L235E) sequence of SEQ ID NO: 14, the human IgG4 YTE sequence of SEQ ID NO: 15, the human IgG4 YTE KiH Hole sequence of SEQ ID NO: 16, and the human IgG4 YTE KiH Knob sequence of SEQ ID NO: 17.

In some embodiments, the antibody comprises a heavy chain constant region comprising the amino acid sequence of SEQ ID NO: 5.

In some embodiments, the antibody comprises a light chain constant region comprising the amino acid sequence of SEQ ID NO: 20.

In some embodiments, the antibody comprises a heavy chain constant region comprising the amino acid sequence of SEQ ID NO: 5 and a light chain constant region comprising the amino acid sequence of SEQ ID NO: 20.

The anti-TrkA antibodies or antigen-binding fragments thereof, as disclosed herein, can exhibit one or more of the following characteristics:
(i) a dissociation constant (KD) for binding human TrkA ranging from about 0.1 nM to about 1,000 nM, preferably about 0.1 nM to about 10 nM, as determined by surface plasmon resonance; and/or
(ii) a half-maximal effective concentration (EC₅₀) value for binding human TrkA ranging from about 10 nM to about 1,000 nM, preferably about 100 nM to about 500 nM;

The anti-TrkA antibodies or antigen-binding fragments thereof, as disclosed herein, can comprise:
(a) a heavy chain variable region (HCVR) sequence of SEQ ID NO: 27, 29, 33, 35, 37, 39, 41, 43, 45, 47, 49, 51, 53, 57, 59, 61, 63, or 65 or a HCVR sequence at least about 90%, about 95% or about 99% identical thereto, and
(b) a light chain variable region (LCVR) of SEQ ID NO: 28, 30, 32, 34, 36, 38, 40, 42, 44, 46, 48, 50, 52, 54, 56, 58, 60, 62, 64, 66 or a LCVR sequence at least about 90%, about 95% or about 99% identical thereto.

In some embodiments, the anti-TrkA antibodies, or antigen-binding fragments thereof, as disclosed herein, comprise:
(a) a heavy chain variable region (HCVR) sequence of SEQ ID NO: 27, 29 or 49, and
(b) a light chain variable region (LCVR) of SEQ ID NO: 28, 30 or 50.

In some embodiments, the anti-TrkA antibodies, or antigen-binding fragments thereof, comprise a HCVR comprising the amino acid sequence of SEQ ID NO: 27 and a LCVR comprising the amino acid sequence of SEQ ID NO: 28. In some embodiments, the anti-TrkA antibodies, or antigen-binding fragments thereof, comprise a HCVR comprising the amino acid sequence of SEQ ID NO: 29 and a LCVR comprising the amino acid sequence of SEQ ID NO: 30. In some embodiments, the anti-TrkA antibodies, or antigen-binding fragments thereof, comprise a HCVR comprising the amino acid sequence of SEQ ID NO: 49 and a LCVR comprising the amino acid sequence of SEQ ID NO: 50. In some embodiments, the anti-TrkA antibodies, or antigen-binding fragments thereof, can comprise a heavy chain constant region comprising the amino acid sequence selected from the group consisting of: SEQ ID NO: 1; 2; 3; 4; 5; 6; 7; 8; 9; 10; 11; 12; 13; 14; 15; 16; 17 and 18. In some embodiments, the anti-TrkA antibody comprises a light chain which is a human kappa light chain. In some embodiments, the anti-TrkA antibody comprises a light chain which is a human lambda light chain. In some embodiments, the anti-TrkA antibodies, or antigen-binding fragments thereof, can comprise a light chain constant region comprising the amino acid sequence selected from the group consisting of: SEQ ID NO: 19 and SEQ ID NO: 20. In some embodiments, the anti-TrkA antibodies, or antigen-binding fragments thereof, can comprise:
(a) a heavy chain constant region of an amino acid sequence of SEQ ID NO: 1, 2, 3, 4, 5, 6, 7, 8, 9, 10, 11, 12, 13, 14, 15, 16, 17 or 18, and
(b) a light chain constant region of an amino acid sequence of SEQ ID NO: 19 or SEQ ID NO: 20.

In some embodiments, the anti-TrkA antibodies, or antigen-binding fragments thereof, can comprise a heavy chain constant region comprising the amino acid sequence of SEQ ID NO: 5 and a light chain constant region comprising the amino acid sequence of SEQ ID NO: 20.

In some embodiments, the anti-TrkA antibodies, as disclosed herein, are full length antibodies. In some embodiments, the anti-TrkA antibodies are antigen-binding fragments thereof.

In some embodiments, the anti-TrkA antibodies, as disclosed herein, comprises a light chain which is a human kappa light chain.

In some embodiments, the anti-TrkA antibodies or antigen-binding fragments thereof, as disclosed herein, comprises a light chain which is a human lambda light chain.

In one embodiment, the full length anti-TrkA antibodies comprises:
(a) a HCVR comprising the amino acid sequence of SEQ ID NO: 27 and a LCVR comprising the amino acid sequence of SEQ ID NO: 28, and
(b) a heavy chain constant region comprising the amino acid sequence of SEQ ID NO: 5 and a light chain constant region comprising the amino acid sequence of SEQ ID NO: 20.

In one embodiment, the full length anti-TrkA antibodies comprises:
(a) a HCVR comprising the amino acid sequence of SEQ ID NO: 29 and a LCVR comprising the amino acid sequence of SEQ ID NO: 30, and
(b) a heavy chain constant region comprising the amino acid sequence of SEQ ID NO: 5 and a light chain constant region comprising the amino acid sequence of SEQ ID NO: 20.

In one embodiment, the full length anti-TrkA antibodies comprises:
(a) a HCVR comprising the amino acid sequence of SEQ ID NO: 49 and a LCVR comprising the amino acid sequence of SEQ ID NO: 50, and
(b) a heavy chain constant region comprising the amino acid sequence of SEQ ID NO: 5 and a light chain constant region comprising the amino acid sequence of SEQ ID NO: 20.

In one embodiment, the full length anti-TrkA antibodies comprises a heavy chain comprising the amino acid sequence of SEQ ID NO: 77 and a light chain comprising the amino acid sequence of SEQ ID NO: 78.

In an embodiment, the anti-TrkA antibodies or antigen-binding fragments thereof, as disclosed herein, is a monoclonal antibody, preferably a human monoclonal antibody.

Also disclosed herein are compositions comprising any of the disclosed the anti-TrkA antibodies or antigen-binding fragments thereof in combination with at least one pharmaceutically acceptable carrier.

Also disclosed herein are immunoconjugates comprising any of the disclosed anti-TrkA antibodies or antigen-binding fragments thereof linked to at least one agent. An agent appropriate for treating disease may include a cytotoxic agent such as chemotherapeutic, radiotherapeutic or toxin.

For diagnostic purposes, an appropriate agent may include a detectable label that includes a radioisotope (e.g. for whole body imaging), an enzyme, a fluorescent label and other suitable antibody tag for sample testing.

Also disclosed herein are anti-TrkA antibodies or antigen-binding fragments thereof, that cross-compete for binding to TrkA with a reference antibody, where the reference antibody comprises:
(a) a HCDR1 comprising the amino acid sequence of SEQ ID NO:21,
(b) a HCDR2 comprising the amino acid sequence of SEQ ID NO: 22,
(c) a HCDR3 comprising the amino acid sequence of SEQ ID NO: 23;
(d) a LCDR1 comprising the amino acid sequence of SEQ ID NO:24,
(e) a LCDR2 comprising the amino acid sequence of SEQ ID NO:25, and
(f) a LCDR3 comprising the amino acid sequence of SEQ ID NO:26.

In some embodiments, the reference antibody comprises:
a heavy chain complementarity determining region 1 (HCDR1) comprising the amino acid sequence of SEQ ID NO: 71, a HCDR2 comprising the amino acid sequence of SEQ ID NO: 72, a HCDR3 comprising the amino acid sequence of SEQ ID NO: 73, a light chain complementarity determining region 1 (LCDR1) comprising the amino acid sequence of SEQ ID NO: 74, a LCDR2 comprising the amino acid sequence of SEQ ID NO: 75, and a LCDR3 comprising the amino acid sequence of SEQ ID NO: 76.
wherein the HCDR1 is numbered according to AbM and the remaining CDRs are numbered according to Kabat.

In some embodiments, the reference antibody comprises:
(a) a heavy chain complementarity determining region (HCDR1) comprising the amino acid sequence of SEQ ID NO: 21,
(b) a HCDR2 comprising the amino acid sequence of SEQ ID NO: 22,
(c) a HCDR3 comprising the amino acid sequence of SEQ ID NO: 23;
(d) a light chain complementarity determining region (LCDR1) comprising the amino acid sequence of SEQ ID NO: 24,
(e) a LCDR2 comprising the amino acid sequence of SEQ ID NO: 25, and
(f) a LCDR3 comprising the amino acid sequence of SEQ ID NO: 26.

In some embodiments, in the reference antibody:
the Xb residue in SEQ ID NO: 21 is V or I,
the Xc residue in SEQ ID NO: 21 is G or S,
the Xd residue in SEQ ID NO: 22 is T or R,
the Xg residue in SEQ ID NO: 23 is F or W,
the Xh residue in SEQ ID NO: 23 is R or Y,
the X1 residue in SEQ ID NO: 24 is D, G, A, or L,
the X2 residue in SEQ ID NO: 24 is S or A,
the X4 residue in SEQ ID NO: 25 is N or G,
the X5 residue in SEQ ID NO: 25 is N or K,
the X6 residue in SEQ ID NO: 26 is N or A,
the X7 residue in SEQ ID NO: 26 is V, D, or A,
the X8 residue in SEQ ID NO: 26 is S or Y, and
the X9 residue in SEQ ID NO: 26 is G or S.

In some embodiments, in the reference antibody:
the Xb residue in SEQ ID NO: 21 is I,
the Xc residue in SEQ ID NO: 21 is S,
the Xd residue in SEQ ID NO: 22 is R,
the Xg residue in SEQ ID NO: 23 is W,
the Xh residue in SEQ ID NO: 23 is Y,
the X1 residue in SEQ ID NO: 24 is D,
the X2 residue in SEQ ID NO: 24 is A,
the X4 residue in SEQ ID NO: 25 is G,
the X5 residue in SEQ ID NO: 25 is N,
the X6 residue in SEQ ID NO: 26 is A,
the X7 residue in SEQ ID NO: 26 is A,
the X8 residue in SEQ ID NO: 26 is S, and
the X9 residue in SEQ ID NO: 26 is S.

In some embodiments, in the reference antibody:
the Xb residue in SEQ ID NO: 21 is V,
the Xc residue in SEQ ID NO: 21 is G,
the Xd residue in SEQ ID NO: 22 is T,
the Xg residue in SEQ ID NO: 23 is F,
the Xh residue in SEQ ID NO: 23 is Y,
the X1 residue in SEQ ID NO: 24 is D,
the X2 residue in SEQ ID NO: 24 is S,
the X4 residue in SEQ ID NO: 25 is N,
the X5 residue in SEQ ID NO: 25 is N,
the X6 residue in SEQ ID NO: 26 is N,
the X7 residue in SEQ ID NO: 26 is V,
the X8 residue in SEQ ID NO: 26 is S, and
the X9 residue in SEQ ID NO: 26 is G.

In some embodiments, in the reference antibody:
the Xb residue in SEQ ID NO: 21 is I,
the Xc residue in SEQ ID NO: 21 is S,
the Xd residue in SEQ ID NO: 22 is R,
the Xg residue in SEQ ID NO: 23 is W,
the Xh residue in SEQ ID NO: 23 is Y,
the X1 residue in SEQ ID NO: 24 is D,
the X2 residue in SEQ ID NO: 24 is S,
the X4 residue in SEQ ID NO: 25 is N,
the X5 residue in SEQ ID NO: 25 is N,
the X6 residue in SEQ ID NO: 26 is N,
the X7 residue in SEQ ID NO: 26 is D,
the X8 residue in SEQ ID NO: 26 is S, and
the X9 residue in SEQ ID NO: 26 is S.

In some embodiments, the reference antibody comprises an IgG1 heavy chain constant region, an IgG2 heavy chain constant region, or an IgG4 heavy chain constant region. In some embodiments, the reference antibody comprises an IgG1 heavy chain constant region that comprises an L235A substitution and a G237A substitution.

In some embodiments, the reference antibody comprises a heavy chain constant region comprising the amino acid sequence selected from the group consisting of: SEQ ID NO: 1; 2; 3; 4; 5; 6; 7; 8; 9; 10; 11; 12; 13; 14; 15; 16; 17 and 18. In some embodiments, the reference antibody comprises a light chain which is a human kappa light chain. In some embodiments, the reference antibody comprises a light chain which is a human lambda light chain. In some embodiments, the reference antibody can comprise a light chain constant region comprising the amino acid sequence selected from the group consisting of: SEQ ID NO: 19 and SEQ ID NO: 20. In some embodiments, the reference antibody comprises:
(a) a heavy chain constant region of an amino acid sequence of SEQ ID NO: 1, 2, 3, 4, 5, 6, 7, 8, 9, 10, 11, 12, 13, 14, 15, 16, 17 or 18, and
(b) a light chain constant region of an amino acid sequence of SEQ ID NO: 19 or SEQ ID NO: 20.

In some embodiments, the reference antibody comprises a heavy chain constant region comprising the amino acid sequence of SEQ ID NO: 5 and a light chain constant region comprising the amino acid sequence of SEQ ID NO: 20.

In some embodiments, the reference antibody comprises a heavy chain (HC) comprising the amino acid sequence of SEQ ID NO: 77. In some embodiments, the reference antibody comprises a light chain (LC) comprising the amino acid sequence of SEQ ID NO: 78.

In some embodiments, the reference antibody comprises a heavy chain (HC) comprising the amino acid sequence of SEQ ID NO: 77 and a light chain (LC) comprising the amino acid sequence of SEQ ID NO: 78.

Also disclosed are nucleic acid molecules encoding any of the disclosed anti-TrkA antibodies or antigen-binding fragments thereof.

Also disclosed are expression vectors comprising the nucleic acid molecules encoding any of the disclosed anti-TrkA antibodies or antigen-binding fragments thereof.

Also disclosed are host cells comprising the nucleic acid molecules encoding any of the disclosed anti-TrkA antibodies or antigen-binding fragments thereof, wherein a host cell capable of producing a human being is excluded. Further disclosed are host cells comprising the expression vectors that comprise the nucleic acid molecules encoding any of the disclosed anti-TrkA antibodies or antigen-binding fragments thereof, wherein a host cell capable of producing a human being is excluded.

Also disclosed are transgenic animals comprising human immunoglobulin heavy and light chain transgenes, wherein the animal expresses any of the disclosed anti-TrkA antibodies or antigen-binding fragments thereof.

Also provide disclosed are hybridomas prepared from a transgenic animal, wherein the hybridoma produces any of the disclosed anti-TrkA antibodies or antigen-binding fragments thereof.

Also disclosed are uses of the anti-TrkA antibodies or antigen-binding fragments thereof, as disclosed herein, for treating disorders in which neuronal dysfunction is associated with the disease pathology, (for example, wherein the neuronal dysfunction is due to impaired neuronal growth, repair, restoration, or regeneration), preferably wherein the anti-TrkA antibodies or antigen-binding fragments thereof bind to and activate TrkA thereby treating the pathology.

Also disclosed herein are uses of any of the herein disclosed anti-TrkA antibodies or antigen-binding fragments thereof for promoting neurite regeneration in a subject in need thereof.

Also disclosed are methods of treating disorders in which neuronal dysfunction is associated with the disease pathology, (for example, wherein the neuronal dysfunction is due to impaired neuronal growth, repair, restoration, or regeneration), comprising administering a therapeutically effective amount of any of the disclosed anti-TrkA antibodies, or antigen-binding fragments thereof, or any of the disclosed compositions, to the subject to thereby treat the disorder.

Also disclosed herein are methods of promoting neurite regeneration in a subject in need thereof.

Also disclosed are uses of an anti-TrkA antibodies or antigen-binding fragments thereof, as disclosed herein, in the manufacture of a medicament for treating disorders in which neuronal dysfunction is associated with the disease pathology, (e.g, wherein the neuronal dysfunction is due to impaired neuronal growth, repair, restoration, or regeneration), preferably wherein the anti-TrkA antibodies or antigen-binding fragments thereof are capable of binding to and activating TrkA to treat the pathology.

Also disclosed are uses of any of the herein disclosed anti-TrkA antibodies or antigen-binding fragments thereof in the manufacture of a medicament for promoting neurite regeneration in a subject in need thereof.

In an embodiment, the disorder of neuronal dysfunction may be associated with diminished neuronal growth when compared to neuronal growth in a subject not suffering from the disorder of neuronal dysfunction (i.e. a healthy subject). In an embodiment, the treatment of a disorder of neuronal dysfunction may alleviate one or more symptoms of the disorder such as pain, paresthesia, numbness, dysesthesia, hyperpathia, anesthesia or hypoesthesia (e.g. including reduction or loss of thermal or touch sensation).

Preferably, the disorder of neuronal dysfunction is associated with impaired neuronal restoration and/or is associated with peripheral neuropathy. More preferably, the disorder of neuronal dysfunction is one or more of:
diabetic peripheral neuropathy,
chemotherapy-induced peripheral neuropathy,
small fiber neuropathy,
nerve injury (e.g. traumatic nerve injury or post-surgical nerve injury),
wound healing (e.g. diabetic foot ulcers), or
optic neuropathies (e.g. glaucoma etc.).

The nerve injury can be a traumatic nerve injury or a post-surgical nerve injury.

The wound healing can be healing of diabetic foot ulcers.

The optic neuropathy can be glaucoma.

Further examples of the antibodies, or antigen-binding fragments thereof, and uses are disclosed below. However, it should be noted that the antibodies, or antigen-binding fragments thereof, and uses should not be limited to these examples, and that the antibodies, or antigen-binding fragments thereof, and uses are susceptible to variations, modifications and/or additions other than those specifically disclosed, and it is to be understood that the antibodies, or antigen-binding fragments thereof, and uses include all such variations, modifications and/or additions which fall within the scope of the claims.

### EMBODIMENTS

The following list of embodiments is intended to complement, rather than displace or supersede, the previous descriptions.

Embodiment 1: An anti-tropomyosin receptor kinase A (TrkA) antibody, or an antigen-binding fragment thereof, comprising:
a heavy chain complementarity determining region 1 (HCDR1) comprising the amino acid sequence of SEQ ID NO: 71, a HCDR2 comprising the amino acid sequence of SEQ ID NO: 72, a HCDR3 comprising the amino acid sequence of SEQ ID NO: 73, a light chain complementarity determining region 1 (LCDR1) comprising the amino acid sequence of SEQ ID NO: 74, a LCDR2 comprising the amino acid sequence of SEQ ID NO: 75, and a LCDR3 comprising the amino acid sequence of SEQ ID NO: 76.

Embodiment 2: The anti-TrkA antibody, or antigen-binding fragment thereof, of embodiment 1, comprising a heavy chain variable region (VH) comprising the amino acid sequence of SEQ ID NO: 49 and a light chain variable region (VL) comprising the amino acid sequence of SEQ ID NO: 50.

Embodiment 3: The anti-TrkA antibody, or antigen-binding fragment thereof, of embodiment 1 or 2, wherein the antibody is a monoclonal antibody.

Embodiment 4: The anti-TrkA antibody, or antigen-binding fragment thereof, of any one of the previous embodiments, wherein the antibody is a human antibody.

Embodiment 5: The anti-TrkA antibody, or antigen-binding fragment thereof, of any one of the previous embodiments, wherein the antigen-binding fragment is a F(ab′)2.

Embodiment 6: The anti-TrkA antibody, or antigen-binding fragment thereof, of any one of the previous embodiments, wherein the antigen-binding fragment is not a Fab.

Embodiment 7: The anti-TrkA antibody, or antigen-binding fragment thereof, of any one of embodiments 1-4, comprising an IgG1 heavy chain constant region, an IgG2 heavy chain constant region, or an IgG4 heavy chain constant region.

Embodiment 8: The anti-TrkA antibody, or antigen-binding fragment thereof, of embodiment 7, comprising an IgG1 heavy chain constant region that comprises an L235A substitution and a G237A substitution.

Embodiment 9: The anti-TrkA antibody, or antigen-binding fragment thereof, of any one of the previous embodiments, comprising a heavy chain (HC) comprising the amino acid sequence of SEQ ID NO: 77 and a light chain (LC) comprising the amino acid sequence of SEQ ID NO: 78.

Embodiment 10: A composition comprising the anti-TrkA antibody, or antigen-binding fragment thereof, of any one of the previous embodiments and a pharmaceutically acceptable carrier.

Embodiment 11: A nucleic acid molecule encoding the anti-TrkA antibody, or antigen-binding fragment thereof, of any one of embodiments 1-9.

Embodiment 12: An expression vector comprising the nucleic acid molecule of embodiment 11.

Embodiment 13: A host cell comprising the nucleic acid molecule according to embodiment 11.

Embodiment 14: A host cell comprising the expression vector of embodiment 12.

Embodiment 15: A method of promoting neurite regeneration in a subject in need thereof or treating a neuronal dysfunction in a subject in need thereof, the method comprising:

administering a therapeutically effective amount of the anti-TrkA antibody, or antigen-binding fragment thereof, of any one of embodiments 1-9, or the composition of embodiment 10 to the subject to thereby promote the neurite regeneration or treat the neuronal dysfunction.

Embodiment 16: The method of embodiment 15, wherein the neuronal dysfunction is:
a diabetic peripheral neuropathy,
a chemotherapy-induced peripheral neuropathy,
a small fiber neuropathy,
a nerve injury,
a wound healing, or
an optic neuropathy.

Embodiment 17: The method of embodiment 16, wherein the nerve injury is a traumatic nerve injury or a post-surgical nerve injury.

Embodiment 18: The method of embodiment 16, wherein the wound healing is healing of diabetic foot ulcers.

Embodiment 19: The method of embodiment 16, wherein the optic neuropathy is glaucoma.

Embodiment 20: An anti-tropomyosin receptor kinase A (TrkA) antibody, or antigen-binding fragment thereof, comprising:
(a) a HCDR1 comprising the amino acid sequence of SEQ ID NO: 21,
(b) a HCDR2 comprising the amino acid sequence of SEQ ID NO: 22,
(c) a HCDR3 comprising the amino acid sequence of SEQ ID NO: 23;
(d) a LCDR1 comprising the amino acid sequence of SEQ ID NO: 24,
(e) a LCDR2 comprising the amino acid sequence of SEQ ID NO: 25, and
(f) a LCDR3 comprising the amino acid sequence of SEQ ID NO: 26,
wherein
the Xb residue in SEQ ID NO: 21 is V or I,
the Xc residue in SEQ ID NO: 21 is G or S,
the Xd residue in SEQ ID NO: 22 is T or R,
the Xg residue in SEQ ID NO: 23 is F or W,
the Xh residue in SEQ ID NO: 23 is R or Y,
the X1 residue in SEQ ID NO: 24 is D, G, A or L,
the X2 residue in SEQ ID NO: 24 is S or A,
the X4 residue in SEQ ID NO: 25 is N or G,
the X5 residue in SEQ ID NO: 25 is N or K,
the X6 residue in SEQ ID NO: 26 is N or A,
the X7 residue in SEQ ID NO: 26 is V, D or A,
the X8 residue in SEQ ID NO: 26 is S or Y, and
the X9 residue in SEQ ID NO: 26 is G or S
and wherein the antibody or antigen-binding fragment thereof selectively binds to and activates TrkA and has
(i) a dissociation constant (KD) for binding human TrkA ranging from about 0.1 nM to about 1,000 nM, preferably about 0.1 nM to about 10 nM, as determined by surface plasmon resonance; and/or
(ii) a half-maximal effective concentration (EC₅₀) value for binding human TrkA ranging from about 10 nM to about 1,000 nM, preferably about 100 nM to about 500 nM.

Embodiment 21: The anti-TrkA antibody, or antigen-binding fragment thereof, of embodiment 20, wherein the dissociation constant (KD) for binding human TrkA ranges from about 0.1 nM to about 10 nM.

Embodiment 22: The anti-TrkA antibody, or antigen-binding fragment thereof, of embodiment 20 or 21, wherein the half-maximal effective concentration (EC₅₀) value for binding human TrkA ranges from about 100 nM to about 500 nM.

Embodiment 23: The anti-TrkA antibody, or antigen-binding fragment thereof, of any one of embodiments 20-22, wherein:
the Xb residue in SEQ ID NO: 21 is I,
the Xc residue in SEQ ID NO: 21 is S,
the Xd residue in SEQ ID NO: 22 is R,
the Xg residue in SEQ ID NO: 23 is W,
the Xh residue in SEQ ID NO: 23 is Y,
the X1 residue in SEQ ID NO: 24 is D,
the X2 residue in SEQ ID NO: 24 is A,
the X4 residue in SEQ ID NO: 25 is G,
the X5 residue in SEQ ID NO: 25 is N,
the X6 residue in SEQ ID NO: 26 is A,
the X7 residue in SEQ ID NO: 26 is A,
the X8 residue in SEQ ID NO: 26 is S, and
the X9 residue in SEQ ID NO: 26 is S.

Embodiment 24: The anti-TrkA antibody, or antigen-binding fragment thereof, of any one of embodiments 20-23 comprising:
(a) a heavy chain variable region (HCVR) CDR1, CDR2 and CDR3 amino acid sequences derived from an amino acid sequence of SEQ ID NO: 27, 29, 33, 35, 37, 39, 41, 43, 45, 47, 49, 51, 53, 57, 59, 61, 63 or 65, and
(b) a light chain variable region (LCVR) CDR1, CDR2 and CDR3 amino acid sequences derived from an amino acid sequence of SEQ ID NO: 28, 30, 32, 34, 36, 38, 40, 42, 44, 46, 48, 50, 52, 54, 56, 58, 60, 62, 64 or 66,
wherein the CDR sequences are identified by the Kabat, Chothia, AbM or IMGT numbering schemes, or combinations thereof.

Embodiment 25: The anti-TrkA antibody, or antigen-binding fragment thereof, of any one of embodiments 20-24, wherein TrkA activation is measured by one or more of the following:
neurite outgrowth,
dimerization of TrkA,
internalization of TrkA,
phosphorylation of TrkA, or
TrkA signaling (including for example, activation of an intracellular pathways associated with neurite outgrowth and neurite survival, including MAPK pathway and P13K pathways (preferably, as measured by assessing Erk and/or Akt phosphorylation)).

Embodiment 26: The anti-TrkA antibody, or antigen-binding fragment thereof, of embodiment 25, wherein the antibody, or antigen-binding fragment thereof, induces neurite outgrowth in the absence of NGF.

Embodiment 27: The anti-TrkA antibody, or antigen-binding fragment thereof, of any one of embodiments 20-26, wherein the antibody, or antigen-binding fragment thereof, induces a submaximal TrkA biological response and/or submaximal induction of one or more downstream pathways in the absence of NGF (Ab-NGF), when compared to NGF induced activation of TrkA measured in the absence of the antibody (NGF-Ab).

Embodiment 28: The anti-TrkA antibody, or antigen-binding fragment thereof, of embodiment 27, wherein the antibody, or antigen-binding fragment thereof, induces TrkA activation in the absence of NGF (antibody (Ab)-NGF), when compared to a control of NGF induced TrkA activation when measured in the absence of the antibody (NGF-Ab) by at least about 45%, 50%, 55%, 60%, 65%, 70%, 75%, or 80% up to about 85%.

Embodiment 29: The anti-TrkA antibody, or antigen-binding fragment thereof, of any one of embodiments 20-28, wherein the antibody has an EC₅₀ value from about 50 pM to about 100 pM, preferably from about 70 pM to 90 pM as determined by an in vitro neurite outgrowth assay comprising PC12 cells, when compared to a control of NGF absent antibody having an EC₅₀ value from about 1 pM to about 15 pM, preferably from about 5 pM to about 8 pM.

Embodiment 30: The anti-TrkA antibody, or antigen-binding fragment thereof, of any one of embodiments 20-29, wherein the antibody is a human antibody, preferably wherein the humanness score of the anti-TrkA antibody is at least about 90% or 95% up to about 98%.

Embodiment 31: The anti-TrkA antibody, or antigen-binding fragment thereof, of any one of embodiments 20-30, wherein the antibody is an IgG1, an IgG2, an IgG3, an IgG4, an IgM, an IgA1, an IgA2, a secretory IgA, an IgD, or an IgE antibody, preferably an IgG1, IgG2 or IgG4 antibody.

Embodiment 32: The anti-TrkA antibody, or antigen-binding fragment thereof, of any one of embodiments 20-31, comprising:
(a) a heavy chain constant region amino acid sequence of one of SEQ ID NO: 1-18, and
(b) a light chain constant region amino acid sequence of SEQ ID NO: 19 or 20.

Embodiment 33: The anti-TrkA antibody, or antigen-binding fragment thereof, of any one of embodiments 20-32, which exhibits one or more of the following characteristics:
(i) a dissociation constant (KD) for binding human TrkA ranging from about 0.1 nM to about 1,000 nM, preferably about 0.1 nM to about 10 nM, as determined by surface plasmon resonance; and/or
(ii) a half-maximal effective concentration (EC₅₀) value for binding human TrkA ranging from about 10 nM to about 1,000 nM, preferably about 100 nM to about 500 nM.

Embodiment 34: The anti-TrkA antibody, or antigen-binding fragment thereof, of any one of embodiments 20-33, comprising:
(a) a heavy chain variable region (HCVR) sequence of SEQ ID NO: 27, 29, 33, 35, 37, 39, 41, 43, 45, 47, 49, 51, 53, 57, 59, 61, 63 or 65, and
(b) a light chain variable region (LCVR) of SEQ ID NO:28, 30, 32, 34, 36, 38, 40, 42, 44, 46, 48, 50, 52, 54, 56, 58, 60, 62, 64 or 66.

Embodiment 35: The anti-TrkA antibody, or antigen-binding fragment thereof, of embodiment 34, comprising:
(a) a heavy chain variable region (HCVR) sequence of SEQ ID NO: 27, 29 or 49, and
(b) a light chain variable region (LCVR) of SEQ ID NO: 28, 30 or 50.

Embodiment 36: The anti-TrkA antibody, or antigen-binding fragment thereof, of any one of embodiments 20-35, wherein the antibody, or antigen-binding fragment thereof, is a monoclonal antibody.

Embodiment 37: The anti-TrkA antibody, or antigen-binding fragment thereof, of any one of embodiments 20-36, wherein the antibody, or antigen-binding fragment thereof, is a human antibody.

Embodiment 38: A composition comprising the anti-TrkA antibody, or antigen-binding fragment thereof, of any one of embodiments 20-37, and a pharmaceutically acceptable carrier.

Embodiment 39: An anti-TrkA antibody, or antigen-binding fragment thereof, that cross-competes for binding to tropomyosin receptor kinase A (TrkA) with a reference antibody which comprises:
(a) a HCDR1 comprising the amino acid sequence of SEQ ID NO: 21,
(b) a HCDR2 comprising the amino acid sequence of SEQ ID NO: 22,
(c) a HCDR3 comprising the amino acid sequence of SEQ ID NO: 23;
(d) a LCDR1 comprising the amino acid sequence of SEQ ID NO: 24,
(e) a LCDR2 comprising the amino acid sequence of SEQ ID NO: 25, and
(f) a LCDR3 comprising the amino acid sequence of SEQ ID NO: 26.

Embodiment 40: A nucleic acid molecule encoding the anti-TrkA antibody, or antigen-binding fragment thereof, of any one of embodiments 20-37 or 39.

Embodiment 41: An expression vector comprising the nucleic acid molecule of embodiment 40.

Embodiment 42: A host cell comprising the nucleic acid molecule of embodiment 40, or the expression vector of embodiment 41, wherein a host cell capable of producing a human being is excluded.

Embodiment 43: A transgenic animal comprising human immunoglobulin heavy and light chain transgenes, wherein the animal expresses the anti-TrkA antibody, or antigen-binding fragment thereof, of any one of embodiments 20-37 or 39.

Embodiment 44: A hybridoma prepared from a transgenic mouse, wherein the hybridoma produces the anti-TrkA antibody, or antigen-binding fragment thereof, of any one of embodiments 20-37 or 39.

Embodiment 45: Use of the anti-TrkA antibody, or antigen-binding fragment thereof, of any one of embodiments 20-37 or 39 for promoting neurite regeneration in a subject in need thereof or treating disorders in which neuronal dysfunction is associated with the disease pathology in a subject in need thereof, wherein the anti-TrkA antibody binds to and activates TrkA thereby treating the pathology.

Embodiment 46: A method of promoting neurite regeneration in a subject in need thereof or treating disorders in which neuronal dysfunction is associated with the disease pathology in a subject in need thereof, comprising administering an effective amount of the anti-TrkA antibody, or antigen-binding fragment thereof, of any one of embodiments 20-37 or 39, wherein the anti-TrkA antibody binds to and activates TrkA thereby treating the pathology.

Embodiment 47: Use of the anti-TrkA antibody, or antigen-binding fragment thereof, of any one of embodiments 20-37 and 39 in the manufacture of a medicament for promoting neurite regeneration in a subject in need thereof or treating disorders in which neuronal dysfunction is associated with the disease pathology in a subject in need thereof, wherein the anti-TrkA antibody is capable of binding TrkA and activating TrkA thereby treating the pathology.

Embodiment 48: An anti-TrkA antibody, or antigen-binding fragment thereof, of any one of embodiments 20-37 or 39 for use in promoting neurite regeneration in a subject in need thereof or treating disorders in which neuronal dysfunction is associated with the disease pathology in a subject in need thereof, preferably wherein the anti-TrkA antibody binds to and activates TrkA thereby treating the pathology.

Embodiment 49: The use according to embodiment 45 or 47, the method according to embodiment 46, or the antibody according to embodiment 48, wherein the disorder of neuronal dysfunction is one or more of:
diabetic peripheral neuropathy,
chemotherapy-induced peripheral neuropathy,
small fiber neuropathy,
nerve injury (e.g. traumatic nerve injury or post-surgical nerve injury),
wound healing (e.g. diabetic foot ulcers), or
optic neuropathies (e.g. glaucoma etc.).

Embodiment 1A: An anti-tropomyosin receptor kinase A (TrkA) antibody, comprising:
(a) a heavy chain variable region (HCVR) CDR1 comprising the amino acid sequence as set forth in SEQ ID NO:21,
(b) a HCVR CDR2 comprising the amino acid sequence as set forth in SEQ ID NO: 22,
(c) a HCVR CDR3 comprising the amino acid sequence as set forth in SEQ ID NO: 23;
(d) a light chain variable region (LCVR) CDR1 comprising the amino acid sequence as set forth in SEQ ID NO:24,
(e) a LCVR CDR2 comprising the amino acid sequence of SEQ ID NO:25, and
(f) a LCVR CDR3 comprising the amino acid sequence of SEQ ID NO:26.

Embodiment 2A: The antibody according to embodiment 1A, wherein:
the Xb residue in SEQ ID NO: 21 is V or I,
the Xc residue in SEQ ID NO: 21 is G or S,
the Xd residue in SEQ ID NO: 22 is T or R,
the Xg residue in SEQ ID NO: 23 is F or W,
the Xh residue in SEQ ID NO: 23 is R or Y,
the X1 residue in SEQ ID NO: 24 is D, G, A or L,
the X2 residue in SEQ ID NO: 24 is S or A,
the X4 residue in SEQ ID NO: 25 is N or G,
the X5 residue in SEQ ID NO: 25 is N or K,
the X6 residue in SEQ ID NO: 26 is N or A,
the X7 residue in SEQ ID NO: 26 is V, D or A,
the X8 residue in SEQ ID NO: 26 is S or Y, and
the X9 residue in SEQ ID NO: 26 is G or S.

Embodiment 3A: The antibody according to embodiment 1A or 2A, wherein:
the Xb residue in SEQ ID NO: 21 is I,
the Xc residue in SEQ ID NO: 21 is S,
the Xd residue in SEQ ID NO: 22 is R,
the Xg residue in SEQ ID NO: 23 is W,
the Xh residue in SEQ ID NO: 23 is Y,
the X1 residue in SEQ ID NO: 24 is D,
the X2 residue in SEQ ID NO: 24 is A,
the X4 residue in SEQ ID NO: 25 is G,
the X5 residue in SEQ ID NO: 25 is N,
the X6 residue in SEQ ID NO: 26 is A,
the X7 residue in SEQ ID NO: 26 is A,
the X8 residue in SEQ ID NO: 26 is S, and
the X9 residue in SEQ ID NO: 26 is S.

Embodiment 4A: An anti-tropomyosin receptor kinase A (TrkA) antibody comprising:
(a) a heavy chain variable region (HCVR) CDR1, CDR2 and CDR3 amino acid sequences derived from an amino acid sequence set forth in SEQ ID NO: 27, 29, 33, 35, 37, 39, 41, 43, 45, 47, 49, 51, 53, 57, 59, 61, 63 or 65, and
(b) a light chain variable region (LCVR) CDR1, CDR2 and CDR3 amino acid sequences derived from an amino acid sequence set forth in SEQ ID NO: 28, 30, 32, 34, 36, 38, 40, 42, 44, 46, 48, 50, 52, 54, 56, 58, 60, 62, 64 or 66,
wherein the CDR sequences are identified by the Kabat, Chothia, AbM or IMGT numbering schemes, or combinations thereof.

Embodiment 5A: The anti-TrkA antibody according to any one of the previous embodiments, wherein the antibody binds to and activates TrkA.

Embodiment 6A: The anti-TrkA antibody according to embodiment 3A, wherein TrkA activation is measured by one or more of the following:
neurite outgrowth,
dimerization of TrkA,
internalization of TrkA,
phosphorylation of TrkA, or
TrkA signaling (including for example, activation of an intracellular pathways associated with neurite outgrowth and neurite survival, including MAPK pathway and P13K pathways (preferably, as measured by assessing Erk and/or Akt phosphorylation)).

Embodiment 7A: The anti-TrkA antibody according to any one of the previous embodiments, wherein the antibody induces neurite outgrowth in the absence of NGF.

Embodiment 8A: The anti-TrkA antibody according to any one of embodiments 1A-6A, wherein the antibody induces a submaximal TrkA biological response and/or submaximal induction of one or more downstream pathways in the absence of NGF (Ab-NGF), when compared to NGF induced activation of TrkA measured in the absence of the antibody (NGF-Ab).

Embodiment 9A: The anti-TrkA antibody according to embodiment 8A, wherein the antibody induces TrkA activation in the absence of NGF (antibody (Ab)-NGF), when compared to a control of NGF induced TrkA activation when measured in the absence of the antibody (NGF-Ab) by at least about 45%, 50%, 55%, 60%, 65%, 70%, 75%, or 80% up to about 85%:

Embodiment 10A: The anti-TrkA antibody according to embodiment 8A or 9A, wherein the antibody has an EC₅₀ value from about 50 pM to about 100 pM, preferably from about 70 pM to 90 pM as determined by an in vitro neurite outgrowth assay comprising PC12 cells, when compared to a control of NGF absent antibody having an EC₅₀ value from about 1 pM to about 15 pM, preferably from about 5 pM to about 8 pM.

Embodiment 11A: The anti-TrkA antibody according to any one of the previous embodiments, wherein the antibody is a human antibody, preferably wherein the humanness score of the anti-TrkA antibody is at least about 90% or 95% up to about 98%.

Embodiment 12A: The anti-TrkA antibody according to any one of the previous embodiments, wherein the antibody is an IgG1, an IgG2, an IgG3, an IgG4, an IgM, an IgA1, an IgA2, a secretory IgA, an IgD, and an IgE antibody, preferably an IgG1, IgG2 or IgG4 antibody.

Embodiment 13A: The anti-TrkA antibody according to any one of the previous embodiments comprising:
(a) a heavy chain constant region amino acid sequence as set forth in one of SEQ ID NOS: 1-18, and
(b) a light chain constant region amino acid sequence as set forth in SEQ ID NO: 19 or 20.

Embodiment 14A: The anti-TrkA antibody according to any one of the previous embodiments, which exhibits one or more of the following characteristics:
(i) a dissociation constant (KD) for binding human TrkA ranging from about 0.1 nM to about 1,000 nM, preferably about 0.1 nM to about 10 nM, as determined by surface plasmon resonance; and/or
(ii) a half-maximal effective concentration (EC₅₀) value for binding human TrkA ranging from about 10 nM to about 1,000 nM, preferably about 100 nM to about 500 nM;

Embodiment 15A: The anti-TrkA antibody according to any one of the previous embodiments which comprises:

(a) a heavy chain variable region (HCVR) sequence as set forth in SEQ ID NO: 27, 29, 33, 35, 37, 39, 41, 43, 45, 47, 49, 51, 53, 57, 59, 61, 63 or 65, or a HCVR sequence at least about 90%, about 95% or about 99% identical thereto, and
(b) a light chain variable region (LCVR) set forth in SEQ ID NO:28, 30, 32, 34, 36, 38, 40, 42, 44, 46, 48, 50, 52, 54, 56, 58, 60, 62, 64 or 66, or a LCVR sequence at least about 90%, about 95% or about 99% identical thereto.

Embodiment 16A: The anti-TrkA antibody according to embodiment 15A which comprises:
(a) a heavy chain variable region (HCVR) sequence as set forth in SEQ ID NO: 27, 29 or 49, and
(b) a light chain variable region (LCVR) set forth in SEQ ID NO: 28, 30 or 50.

Embodiment 17A: The anti-TrkA antibody according to any one of the previous embodiments, wherein the antibody is a full length antibody or antigen-binding fragment thereof.

Embodiment 18a: The antibody according to any one of the preceding embodiments, wherein the antibody is a monoclonal antibody.

Embodiment 19A: The antibody according to any one of the preceding embodiments, wherein the antibody is a human antibody.

Embodiment 20A: A composition comprising the anti-TrkA antibody according to any one of the previous embodiments, and a pharmaceutically acceptable carrier.

Embodiment 21A: An immunoconjugate comprising the anti-TrkA antibody according to any one of embodiments 1A-19A, linked to a therapeutic agent.

Embodiment 22A: A bispecific molecule comprising the anti-TrkA antibody according to any one of embodiments 1A-19A, linked to a second functional moiety having a different binding specificity than said antibody, or antigen-binding portion thereof.

Embodiment 23A: An anti-TrkA antibody that cross-competes for binding to tropomyosin receptor kinase A (TrkA) with a reference antibody which comprises:
(a) a heavy chain variable region (HCVR) CDR1 comprising the amino acid sequence as set forth in SEQ ID NO:21,
(b) a HCVR CDR2 comprising the amino acid sequence as set forth in SEQ ID NO: 22,
(c) a HCVR CDR3 comprising the amino acid sequence as set forth in SEQ ID NO: 23;
(d) a light chain variable region (LCVR) CDR1 comprising the amino acid sequence as set forth in SEQ ID NO:24,
(e) a LCVR CDR2 comprising the amino acid sequence of SEQ ID NO:25, and
(f) a LCVR CDR3 comprising the amino acid sequence of SEQ ID NO:26.

Embodiment 24A: A nucleic acid molecule encoding the anti-TrkA antibody according to any one of embodiments 1A-19A.

Embodiment 25A: An expression vector comprising the nucleic acid molecule according to embodiment 24A.

Embodiment 26A: A host cell comprising the nucleic acid molecule according to embodiment 24A, or expression vector of embodiment 25A, wherein a host cell capable of producing a human being is excluded.

Embodiment 27A: A transgenic animal comprising human immunoglobulin heavy and light chain transgenes, wherein the animal expresses the anti-TrkA antibody according to any one of embodiments 1A-19A.

Embodiment 28A: A hybridoma prepared from a transgenic mouse, wherein the hybridoma produces the anti-TrkA antibody according to any one of embodiments 1A-19A.

Embodiment 29A: Use of the anti-TrkA antibody according to any one of embodiments 1A-19A for treating disorders in which neuronal dysfunction is associated with the disease pathology, preferably wherein the anti-TrkA antibody binds to and activates TrkA thereby treating the pathology.

Embodiment 30A: A method of treating disorders in which neuronal dysfunction is associated with the disease pathology, wherein a subject in need is administered an effective amount of the anti-TrkA antibody according to any one of embodiments 1A-19A, and preferably wherein the anti-TrkA antibody binds to and activates TrkA thereby treating the pathology.

Embodiment 31A: Use of the anti-TrkA antibody according to any one of embodiments 1A-19A in the manufacture of a medicament for treating disorders in which neuronal dysfunction is associated with the disease pathology, preferably wherein the anti-TrkA antibody is capable of binding TrkA and activating TrkA thereby treating the pathology.

Embodiment 32A: An anti-TrkA antibody according to any one of embodiments 1A-19A for use in treating disorders in which neuronal dysfunction is associated with the disease pathology, preferably wherein the anti-TrkA antibody binds to and activates TrkA thereby treating the pathology.

Embodiment 33A: The use according to embodiment 29A or 31A, the method according to embodiment 30A, or the antibody according to embodiment 32A, wherein the disorder of neuronal dysfunction is one or more of:
diabetic peripheral neuropathy,
chemotherapy-induced peripheral neuropathy,
small fiber neuropathy,
nerve injury (e.g., traumatic nerve injury or post-surgical nerve injury),
wound healing (e.g., diabetic foot ulcers), or
optic neuropathies (e.g., glaucoma etc.).

### EXAMPLES

The following examples are disclosed to further describe some of the embodiments disclosed herein. The examples are intended to illustrate, not to limit, the disclosed embodiments.

### Example 1—Generation of Anti-TrkA Antibodies

Anti-TrkA antibodies were obtained by genetic immunization of human immunoglobulin transgenic animals. Immune serum was taken at day 24 after 4 immunizations with DNA encoding human TrkA. Sera were tested by flow cytometry using mammalian cells transiently transfected either with the human or the murine TrkA cDNA. Based on a positive serum titer, specific animals were chosen for hybridoma fusion.

Hybridomas positive for binding to human and murine TrkA were used to generate heavy and light chain IgG genes. Human IgG4 was expressed recombinantly in mammalian cells, purified, and screened for binding to human and murine TrkA by whole cell ELISA on cells transfected with human or murine TrkA. From a total of 50 anti-TrkA antibodies, 16 antibodies were selected based on the following criteria: affinity and cross-reactivity by surface plasmon resonance, DiscoverX (Eurofins) dimerization assay and TF1 proliferation assays. The 16 antibodies were further assessed for their ability to induce neurite outgrowth and signaling via the Erk and Akt pathways in the presence and absence of NGF. These assays are disclosed in more detail in the following example.

### Example 2—In Vitro Assessment of Anti-TrkA Antibodies

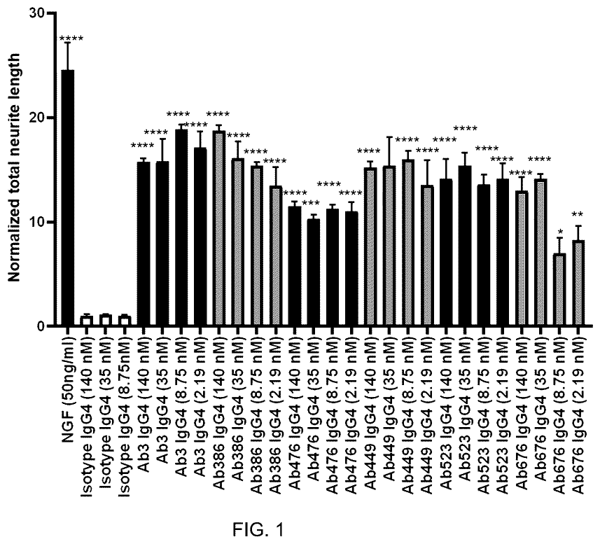

In vitro assays were performed for several anti-TrkA antibodies (Ab3, Ab386, Ab449, Ab476, Ab523 and Ab676) having an IgG4 or IgG2 constant region. For example, the antibodies were assessed for neurite outgrowth (using PC12 cells under standard testing conditions), TrkA receptor dimerization and activation of the Erk and Akt pathway in the absence of NGF. Neurite outgrowth assay results with IgG4 variants are shown in FIG. 1 in the absence of NGF. NGF 50 ng/ml was used as positive control.

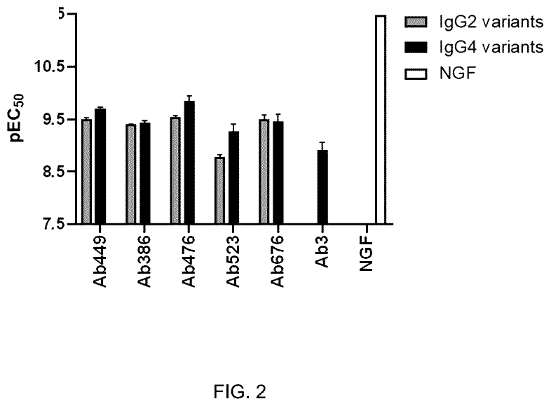

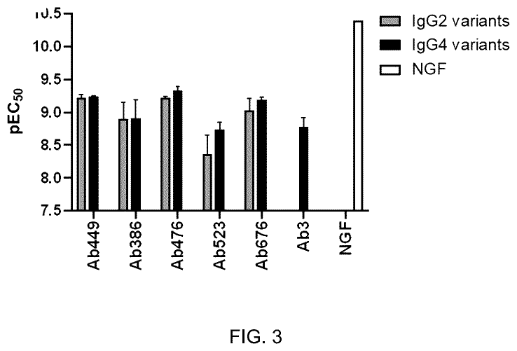

The assay results from assessing phosphorylation of Erk and Akt are shown in FIG. 2 and FIGS. 3, respectively. In FIGS. 2, PC12 cells were stimulated for 15 minutes with various concentrations of the anti-TrkA antibodies (Ab3, Ab386, Ab449, Ab476, Ab523 and Ab676) on IgG2 and IgG4 backbones in the absence of NGF. 2 nM mouse βNGF was used as positive control. Phosphorylated Erk signal was measured using the AlphaLISA SureFire Ultra p-ERK1/2 (Thr202/Tyr204) kit and normalized to total Erk. pEC₅₀ values were taken as the negative log EC₅₀ calculated from the concentration response using the nonlinear fit curve with variable slope four parameter equation in GraphPad Prism (n=2). Data are mean±SEM.

In FIGS. 3, PC12 cells were stimulated for 15 minutes with various concentrations of the anti-TrkA antibodies (Ab3, Ab386, Ab449, Ab476, Ab523 and Ab676) having an IgG2 or IgG4 constant region, in the absence of NGF. 2 nM mouse βNGF was used as a positive control. Phosphorylated Akt signaling was measured using the AlphaLISA SureFire Ultra p-Akt1/2/3 (Thr308) kit and normalized to total Akt. pEC₅₀ values were taken as the negative log EC₅₀ calculated from the concentration response using the nonlinear fit curve with variable slope four parameter equation in GraphPad Prism (n=2). Data are mean±SEM.

### Example 3—Characterization of Further Anti-TrkA Antibodies

Further in vitro functional measurement of 14 additional anti-TrkA antibodies (having IgG4 constant region) was performed to assess neurite outgrowth and activation of the Erk and Akt as disclosed in Example 2. Comparable function in cell-based assays was observed between Ab386 IgG4 and Ab386 IgG1 LAGA in the neurite outgrowth assay, and Erk and Akt pathway activation assays (data not shown).

The potency of the 14 anti-TrkA antibodies in the three screening assays is shown in Table 4.

TABLE 4
In vitro assessment of additional anti-TrkA
antibody variants (IgG4)

| | Neurite | Erk pathway | Akt pathway |
|---|---|---|---|
| Antibody | outgrowth | activation | activation |
| (IgG4) | (pEC₅₀) | (pEC₅₀) | (pEC₅₀) |
| Ab3 | 9.79 | 8.86 | 8.67 |
| Ab386 | 10.09 | 9.23 | 9.00 |
| Ab49 | 10.09 | 9.29 | 9.12 |
| Ab67 | 9.96 | 9.28 | 9.07 |
| Ab70 | 10.04 | 9.21 | 9.05 |
| Ab76 | 9.95 | 9.78 | 9.57 |
| Ab79 | 10.21 | 9.21 | 8.97 |
| Ab82 | 9.99 | 9.14 | 9.09 |
| Ab96 | 9.81 | 10.24 | 9.53 |
| Ab101 | 10.03 | 9.45 | 9.20 |
| Ab119 | 10.2 | 10.18 | 9.25 |
| Ab122 | 9.6 | 9.88 | 9.50 |
| Ab124 | 10.12 | 9.23 | 9.03 |
| Ab142 | 9.97 | 9.54 | 9.53 |
| Ab145 | 10.14 | 9.89 | 9.68 |
| Ab147 | 10.08 | 9.11 | 9.25 |

### Example 4—Analysis of Humanness and Potential Immunogenicity of Anti-TrkA Antibodies

The anti-TrkA antibodies of the present disclosure were engineered to obtain a high humanness score without compromising the high affinity and the antibody functionality as disclosed herein, using the different in vitro functionality assays (e.g. neurite outgrowth, TrkA receptor dimerization and activation of the Erk and Akt pathway). A humanness score, i.e. similarity to human germline sequence, was obtained using various antibody analysis platforms, including IMGT and AbGenesis (Distributed Bio Inc.). Starting with a single input variable region antibody sequence, the closest human germline of the sequence was identified and the percentage of humanness of the variable heavy and light chains was computed using AbGenesis software (Release 4.1). The overall humanness of the antibody was calculated based on average percent sequence identity values of the heavy and light chains.

The humanness scores of some exemplary anti-TrkA antibodies are shown in Table 5.

TABLE 5
Humanness score of
anti-TrkA antibodies

| | Human- | Human- | AVG |
|---|---|---|---|
| | ness | ness | Human- |
| Ab | score VH | score VL | ness score |
| 3 | 92.8 | 95.8 | 93.05 |
| 6 | 90.8 | 67.7 | 93.16 |
| 386 | 95.9 | 98.9 | 96.06 |
| 449 | 95.9 | 98.9 | 95.74 |
| 476 | 95.9 | 97.9 | 95.42 |
| 523 | 91.8 | 97.9 | 95.10 |
| 676 | 92.8 | 94.7 | 95.08 |
| 49 | 94.8 | 96.8 | 95.39 |
| 67 | 94.8 | 96.8 | 95.29 |
| 70 | 94.8 | 95.8 | 95.19 |
| 76 | 94.8 | 94.7 | 95.29 |
| 79 | 94.8 | 95.8 | 95.40 |
| 82 | 95.9 | 94.7 | 95.29 |
| 96 | 94.8 | 95.8 | 95.39 |
| 101 | 94.8 | 96.8 | 95.39 |
| 119 | 94.8 | 95.8 | 95.18 |
| 122 | 94.8 | 94.7 | 95.28 |
| 124 | 94.8 | 96.8 | 95.41 |
| 142 | 94.8 | 95.8 | 95.28 |
| 145 | 94.8 | 94.7 | 95.28 |
| 147 | 94.8 | 96.8 | 95.80 |

The variable regions and the full sequence of the variants were analyzed using a combination of in vitro techniques such as MHC-peptide binding assays and antigen presentation assays, all of which may help predict the probability of peptides derived from the antibody being able to take part in invoking immunogenicity.

### Example 5—Anti-TrkA Antibody Increases Neurite Outgrowth In Vitro-Full Length Antibody Vs. Fragments

To further examine the neurotrophic capabilities of Ab82, PC12 cells were grown under low serum conditions and treated with various concentrations of anti-TrkA antibody Ab82, isotype control antibody or mouse β-NGF. Apart from the full length antibody, a F(ab′)₂ format of Ab82 (the two antigen-binding (Fab) regions of Ab82 linked by disulfide bonds without Fc region, providing a divalent antibody fragment) and a Fab format of Ab82 (one Fab region of Ab82 without Fc region, providing a monovalent antibody fragment) versions of anti-TrkA antibody (e.g. Ab82) and isotype control antibody were included to assess whether divalent binding is needed for function. Live unlabeled neurite outgrowth was quantified over several days using the Incucyte® software (Sartorius AG). The extent of neurite outgrowth was also quantified using the Incucyte® software.

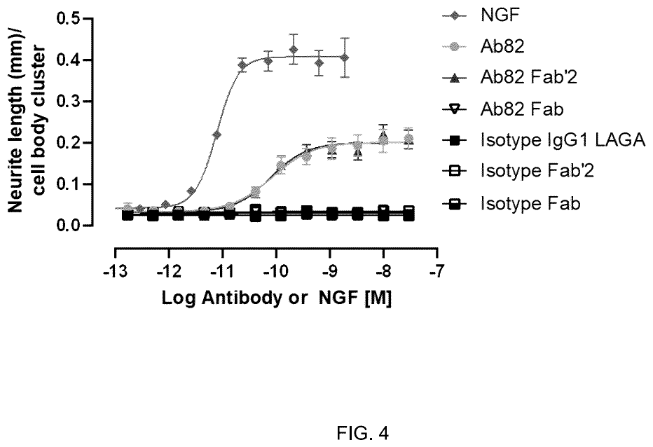

As shown in FIG. 4, at 138 hours post-treatment, Ab82 increased neurite outgrowth, but to a lesser extent than NGF. TrkA receptor dimerization through engagement with a divalent antibody is important for the functional activity of Ab82 since F(ab′)₂ format of Ab 82 had comparable potency to the full-length antibody, whereas Fab format of Ab 82 lacked functional activity. The EC₅₀ values were calculated from the average of 5 experiments: 85.8 pM for Ab82, 78.96 pM for F(ab′)₂ format of Ab 82 and 7.8 pM for murine β-NGF.

### Example 6—Anti-TrkA Antibody Supports Neurite Outgrowth and Cell Health Under NGF Withdrawal Conditions In Vitro

After several days of NGF treatment, PC12 cells terminally differentiate into a neuronal phenotype, become dependent on NGF, and undergo apoptosis after NGF withdrawal. Trophic factor withdrawal-induced reduction in neuronal support and increased apoptosis may mimic what happens in the dysfunctional peripheral nervous system in peripheral neuropathies and neurodegenerative diseases where trophic factor production is compromised.

Investigations were carried out to determine whether Ab82 provides neuronal support on NGF withdrawal. PC12 cells were first differentiated in the presence of NGF for 7 days. Following NGF withdrawal by sequential washes and addition of an anti-NGF antibody, various concentrations of Ab82 were added for 48 hours and high content imaging analysis was performed. Cells were stained with calcein for live cell detection and Hoechst 33342 for nuclear detection. Neurite parameters (total branches, total outgrowth, and total processes) were measured using the ImageXpress® high content system and analyzed using the MetaXpress software.

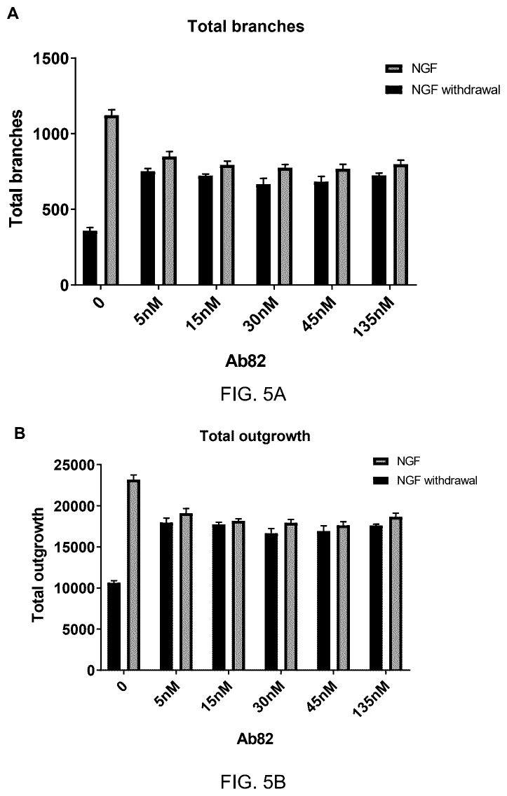

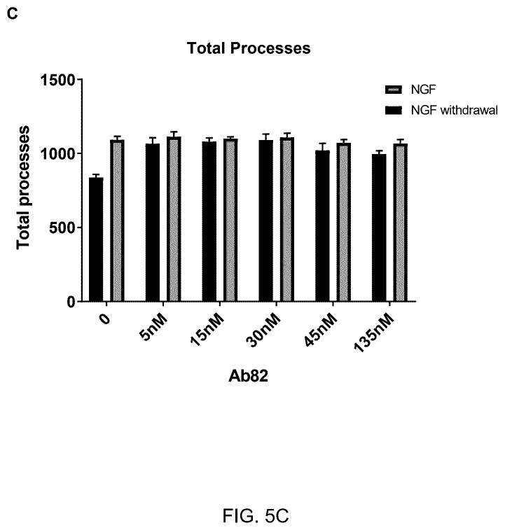

As shown in FIG. 5, NGF withdrawal caused reduction in (A) neurite total branches, (B) outgrowth and (C) total processes. By contrast, treatment with Ab82 results in (A) increased total number of branches, (B) increased outgrowth length, and (C) restored process formation.

### Example 7—Ab82 Increases Axon Regeneration of Primary Sensory Neurons Post-Injury

In peripheral neuropathy conditions, reductions in distal sensory nerve fibers may be related to the functional deficits in sensory signaling that underlie the loss of tactile acuity and increased or decreased pain sensation.

The capability of Ab82 to promote axonal re-growth was assessed in an axotomy model using primary adult mouse dorsal root ganglion (DRG) sensory neurons. Briefly, dissociated DRG neurons isolated from mice were cultivated in microfluidic chambers where an NGF gradient was applied for 5 days to direct axonal growth through microgrooves in the chips from the cell soma to axonal side of the chips. On day 6, the NGF gradient was removed prior to axotomy of the axonal processes. Following axotomy, two concentrations of Ab82 and an isotype control were added to medium on the axonal side and incubated for 48 hours. Axonal regrowth was assessed using an image-based approach evaluating length, number, and branches of crossing axons (stained with tubulin III).

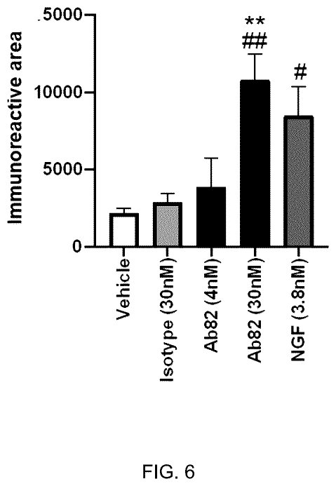

FIG. 6 shows an increase in the total immunoreactive area on the axonal side, representing total length of axons, as well as the number of crossing axons was observed in the 30 nM Ab82 treated wells compared to the isotype control wells. Exogenous NGF treatment also significantly increased axonal regeneration compared to vehicle control treated wells. No significant difference after 4 nM. Ab82 treatment was observed. This data suggests that Ab82 is able to act on injured axons of primary sensory neurons to promote axonal regeneration, albeit to a lower extent when compared to NGF.

### Example 8—Specificity of Ab82

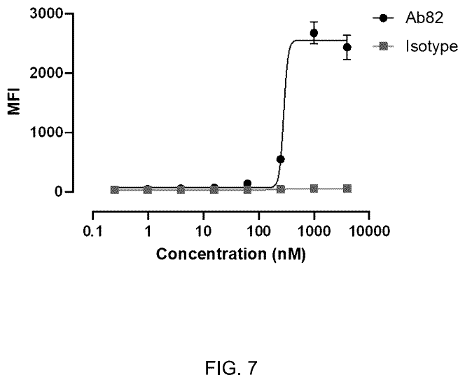

Flow cytometric binding experiments were performed to test binding of Ab82 to native human TrkA expressed endogenously on SH-SY5Y cells. SH-SY5Y cells were incubated with serial dilutions of Ab82 and isotype control KLH IgG1 LAGA, washed and binding was detected using an anti-human IgG Fc B421 secondary while Sytox Red was added to label dead cells. Fluorescence intensity was quantified using flow cytometry and used to calculate median fluorescence intensity (MFI). Ab82 showed positive binding to native human TrkA in a concentration dependent manner as shown in FIG. 7. The EC₅₀ value of binding for Ab82 to native human TrkA is 274.9 nM (calculated from the average of three experiments).

### Example 9—the Binding Affinity of Antibodies to Human TrkA as Measured Using Surface Plasmon Resonance

The affinity of binding to recombinant human TrkA (hTrkA) was determined using surface plasmon resonance (SPR). The antibodies were captured on a protein A chip followed by injection of TrkA as analyte. Kinetic screening was evaluated by fitting the association (Ka) and dissociation (Kd) phases of single-concentration antigen injections to a 1:1 binding model. Examples of the KD, ka, kd values are shown in Table 6.

TABLE 6
Binding affinity of anti-TrkA
antibodies to TrkA

| Ab | K_{D} (M) | kₐ (1/Ms) | k_{d} (1/s) |
|---|---|---|---|
| 3 | 2.10E−09 | 2.93E+05 | 6.14E−04 |
| 386 | 1.15E−09 | 2.66E+05 | 3.05E−04 |
| 449 | 8.75E−10 | 2.79E+05 | 2.44E−04 |
| 476 | 1.25E−09 | 2.40E+05 | 3.00E−04 |
| 523 | 1.30E−09 | 1.99E+05 | 2.58E−04 |
| 676 | 8.15E−10 | 2.65E+05 | 2.16E−04 |
| 49 | 1.78E−09 | 2.64E+05 | 4.72E−04 |
| 67 | 1.91E−09 | 2.50E+05 | 4.77E−04 |
| 70 | 1.63E−09 | 2.65E+05 | 4.32E−04 |
| 76 | 9.43E−10 | | 2.11E−04 |
| 79 | 1.67E−09 | 2.52E+05 | 4.21E−04 |
| 82 | 1.75E−09 | 2.53E+05 | 4.43E−04 |
| 96 | 1.23E−09 | | 2.77E−04 |
| 101 | 1.29E−09 | 2.73E+05 | 3.52E−04 |
| 119 | 1.11E−09 | | 2.65E−04 |
| 122 | 1.45E−09 | | 2.94E−04 |
| 124 | 1.21E−09 | 2.71E+05 | 3.27E−04 |
| 142 | 9.99E−10 | | 2.54E−04 |
| 145 | 1.5E−09 | | 2.99E−04 |
| 147 | 1.19E−09 | 2.64E+05 | 3.14E−04 |

### Example 10-Ab82 does not Bind to Other Members of the Protein Superfamily Related to TrkA as Determined by Surface Plasmon Resonance

Ab82 was screened for binding to the extracellular domain (ECD) of human TrkB (hTrkB), human TrkC (hTrkC), and human p75NTR (hp75NTR). hTrkB-Extracellular Domain-poly C-terminal Histidine tagged (ECD-His), hTrkC-ECD-His, and hp75NTR-His were immobilized on individual flow cells on a CM5 sensor chip using amine coupling. Ab82 and appropriate controls in solution at 25° C. and pH 7.4 were injected across flow cell surfaces at 1 nM. For positive control, neurotrophin 4 (NT4), neurotrophin 3 (NT3), and brain-derived neurotrophic factor (BDNF), natural ligands to TrkB, TrkC, and p75NTR, respectively, were used. For negative isotype control, a hIgG1 LAGA antibody raised against keyhole limpet hemocyanin (KLH) antigen was used.

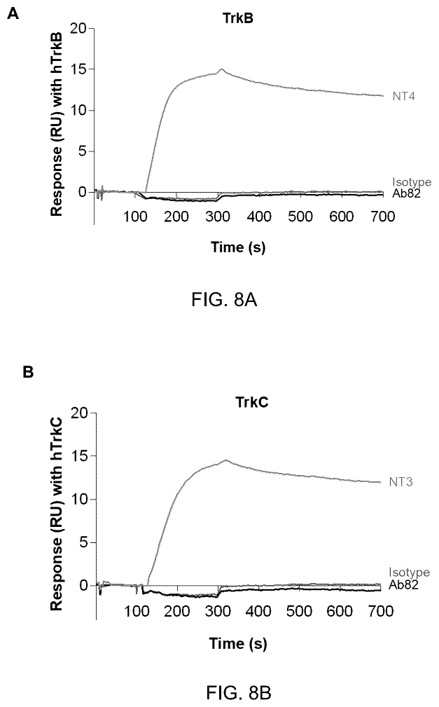

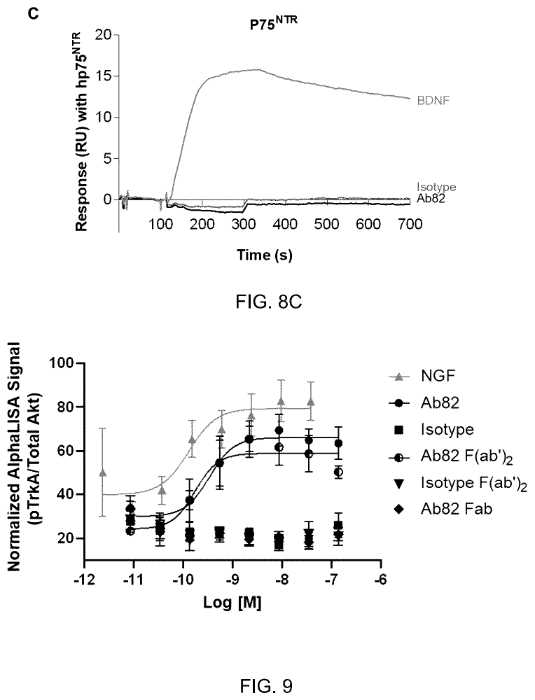

Ab82 and anti-KLH did not bind to hTrkB-ECD-His, hTrkC-ECD-His, or hp75 NTR-His. Only the respective positive control natural ligands bound to the TrkA-related proteins as illustrated in FIG. 8.

### Example 11—Ab82 Increases TrkA Receptor Phosphorylation and Dimerization, Activates MAPK and AKT Signaling Pathways and Undergoes Internalization

TrkA activation involves receptor dimerization which increases TrkA receptor autophosphorylation. To test whether Ab82 affects TrkA receptor phosphorylation, PC12 cells (native rat TrkA expressing cells) were stimulated for 15 minutes with different versions of eAb82, non-targeting antibody or mouse β-NGF at varying concentrations. To determine whether receptor dimerization through engagement with a divalent antibody was important for the functional activity of anti-TrkA antibody (e.g. Ab82), F(ab′)2 format of Ab82 and Fab format of Ab82 were also tested. Phosphorylated TrkA signal was measured using the AlphaLISA SureFire Ultra p-TrkA (Tyr674/675)/p-TrkB (Tyr706/707) kit and normalized to total Akt.

FIG. 9 shows that full length Ab82, F(ab′)2 format of Ab82 and NGF increased TrkA receptor phosphorylation in PC12 cells. Fab format of Ab82 did not increase TrkA receptor phosphorylation indicating the necessity for two antigen-binding regions to cause receptor dimerization which leads to receptor phosphorylation. The EC₅₀ values were calculated from the average of three experiments: 341 pM for Ab82, 175 pM for F(ab′)2 format of Ab 82 and 129 pM for mouse β-NGF.

### Example 12—Ab82 Increases Phosphorylation of Erk and Akt Signaling Pathways

Activation of TrkA induces the Erk and Akt signaling pathways. The Erk pathway contributes to neuronal differentiation and survival and the Akt pathway plays an important role in cell survival.

To test whether Ab82 activates the Erk pathway, PC12 cells (native rat TrkA expressing cells) were stimulated for 15 minutes with different versions of Ab82, non-targeting antibody or mouse β-NGF at varying concentrations. To determine whether receptor dimerization through engagement with a divalent antibody was important for the functional activity of Ab82, F(ab′)2 format of Ab82 and Fab format of Ab82 were also tested. Phosphorylated Erk signal was measured using the AlphaLISA SureFire Ultra p-ERK1/2 (Thr202/Tyr204) kit and normalized to total Erk. Phosphorylated Akt signal was measured using the AlphaLISA SureFire Ultra p-Akt 1/2/3 (Thr308) kit and normalized to total Akt.

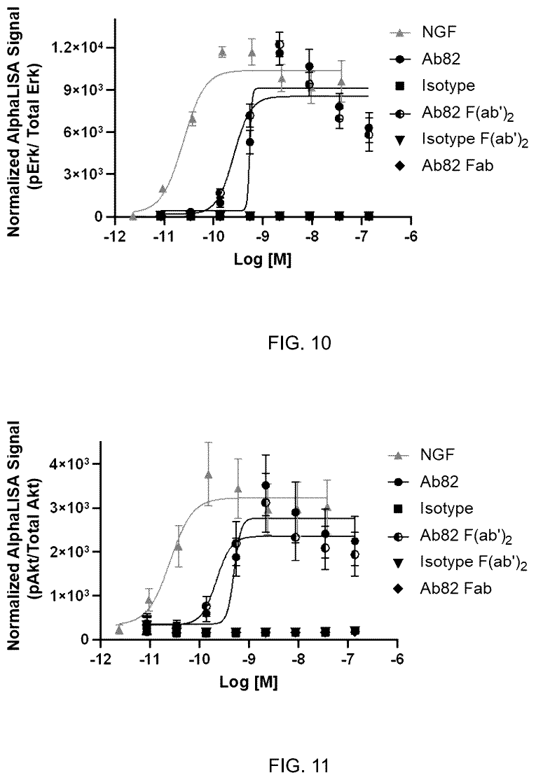

Full length Ab82, F(ab′)2 format of Ab82 and NGF increased Erk phosphorylation (FIG. 10) and Akt phosphorylation (FIG. 11) in PC12 cells. Fab format of Ab82 did not increase Erk or Akt phosphorylation indicating the necessity for two antigen-binding regions to cause receptor dimerization which leads to downstream activation of the Erk and Akt pathways. For Erk phosphorylation, EC₅₀ values were calculated from the average of three experiments (FIG. 10): 540 pM for Ab82, 258 pM for Fab′2 format of Ab82 and 25 pM for mouse β-NGF. For Akt phosphorylation, EC₅₀ values were calculated from the average of 3 experiments (FIG. 11): 501 pM for Ab82, 220 pM for F(ab′)2 format of Ab82 and 25 pM for mouse β-NGF.

### Example 13—Ab82 is Internalized by TrkA Receptor Expressing Cells

A kinetic internalization assay was performed to investigate whether Ab82 undergoes internalization by human, cynomolgus monkey and mouse TrkA-expressing HEK293T cells. Briefly, human, cynomolgus monkey and mouse TrkA constructs were transiently transfected into HEK293S cells and incubated for 48 hours. Following expression of the transiently transfected TrkA constructs, live cell internalization was measured in warm imaging media with Ab82, and isotype control antibodies labelled with pH sensitive Zenon™ pHrodo™ iFL red human IgG labeling reagent. Plates were immediately imaged in the Incucyte (Sartorius) at 37° C., 5% CO2 every 1 hour for 12 hours.

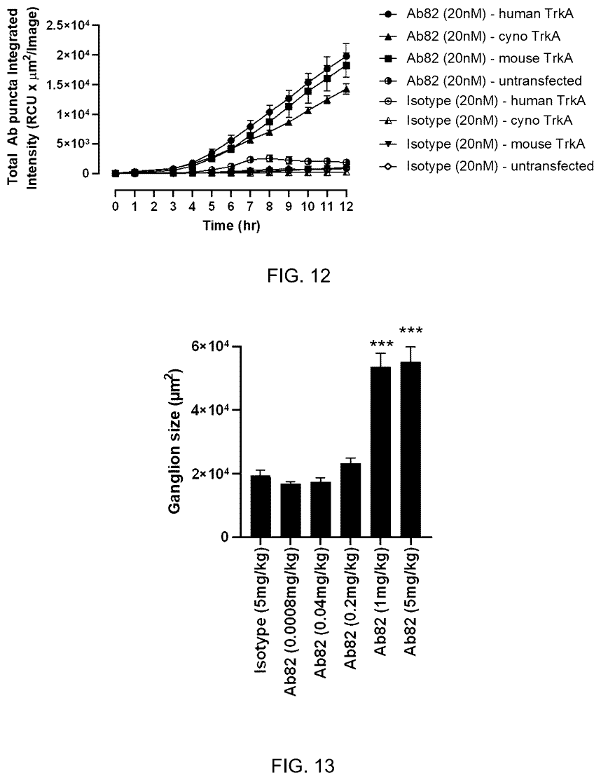

FIG. 12 shows that Ab82 was internalized by cells expressing human, cynomolgus monkey and mouse TrkA. No internalization was observed with isotype control antibody for any tested species TrkA. In addition, Ab82 was not internalized in untransfected cells indicating specific internalization of Ab82 at the TrkA receptors tested. Transient transfection efficiencies were variable among the tested TrkA species thus comparisons of the extent of internalization should not be made between species.

### Example 14—Ab82 has In Vivo Neuronal Support Capabilities (Adult and Neonatal Mice)

To investigate whether Ab82 has in vivo neuronal support capabilities the superior cervical ganglion (SCG) growth model, a model adopted from NGF studies was used where NGF via the TrkA receptor is well known to increase the SCG size.

In adult mice, the effect of Ab82 at various doses (1-15 mg/kg) on superior cervical ganglion size in comparison to isotype control antibody (15 mg/kg) and vehicle (PBS) was assessed. Subcutaneous administration of the test articles in adult mice was carried out once a week for 6 weeks. On week 7, 1 week following the last administration, the SCGs were collected and processed for histology. Machine learning analysis was employed to quantify various parameters associated with the SCG including ganglion size.

FIG. 13 shows the effect of Ab82 at various doses (0.8 μg/kg-5 mg/kg) on SCG size in comparison to isotype control antibody (5 mg/kg) in neonatal mice. Test articles were administered to neonatal mice via the subcutaneous (s.c.) route on days 4, 7, 11, 14 and 18. On postnatal day 19, the SCGs were collected and processed for histology. Machine learning analysis was employed to quantify various parameters associated with the SCG including ganglion size. The figure shows that Ab82 significantly increased SCG size in neonatal mice indicating in vivo neuronal support activity. The effect of Ab82 in neonatal mice is observed to be larger than in adult mice consistent with age-dependent differences in NGF sensitivity.

### Example 15—Ab82 Increased Nerve Fiber Skin Innervation in a Diabetic Peripheral Neuropathy Model

Db/db mice (mutation in the leptin receptor) are a well-characterized and widely used genetic diabetes peripheral neuropathy model. These mice have several deficits associated with clinical diabetes including obesity, chronic hyperglycemia, pancreatic B-cell atrophy, pain, significantly decreased skin intraepidermal nerve fiber density (IENF) and nerve conduction velocities, retinal degeneration and delayed wound healing.

The efficacy of two dose levels of Ab82 (0.5 and 5 mg/kg) compared to isotype control (5 mg/kg) was investigated in the db/db mouse model of diabetic peripheral neuropathy. Briefly, nine-week-old db/db mice were randomized based on body weight, blood glucose and paw withdrawal thresholds into treatment groups. A single undosed group of naïve wildtype C57BKS/J mice was used as a control group. After animal randomization, Ab82, isotype control antibody and saline were dosed via the intraperitoneal (i.p.) route on weeks 9, 10 and 11. Paw tissue was collected at week 11 and processed for IENF measurements with PGP9.5 (pan-neuronal marker) staining.

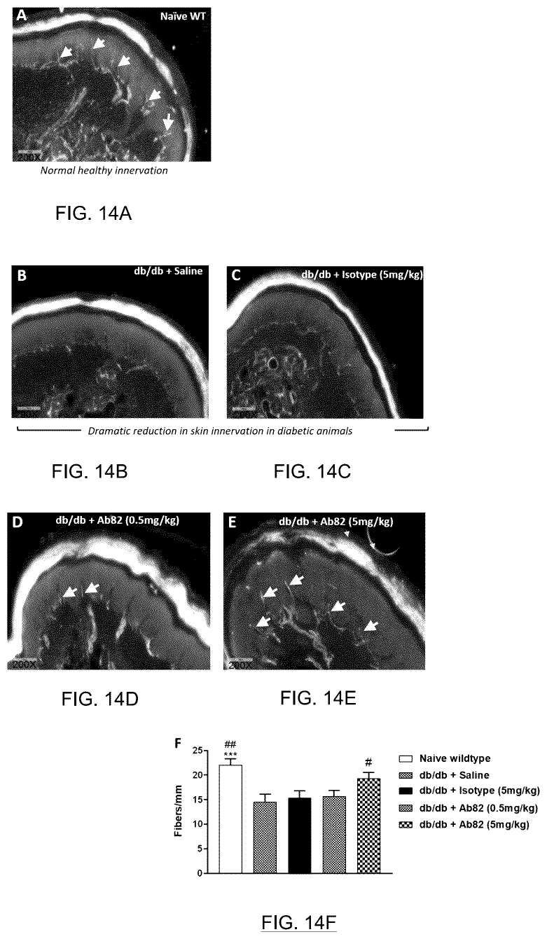

Intra-epidermal nerve fiber density (IENFD) assessments were carried out with PGP9.5 immunostaining of the paw skin harvested at week 11 post-behavioral testing (FIG. 14). PGP9.5 is a pan-neuronal marker used to visualize cutaneous innervation. PGP9.5 immunostaining is the standard for clinical diagnosis of small fiber neuropathies and the methodology for IENFD assessment was followed in this preclinical study. Panel E of FIG. 14 shows that Ab82 (5 mg/kg) treatment in diabetic animals for three weeks significantly increased IENFD compared to diabetic animals treated with vehicle (saline; panel B). Panel F shows IENFD quantification in paw skin-again, Ab82 treatment at 5 mg/kg in diabetic animals for three weeks significantly increased IENFD compared to diabetic animals treated with vehicle saline. Neurorestoration is shown with 5 mg/kg of Ab82 in this study.

### Example 16—Sequences of Anti-TrkA Antibody Variants

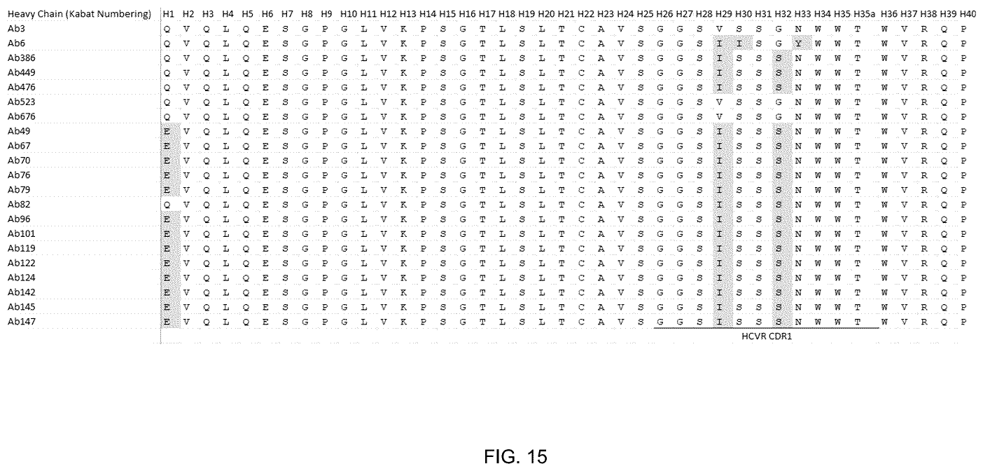

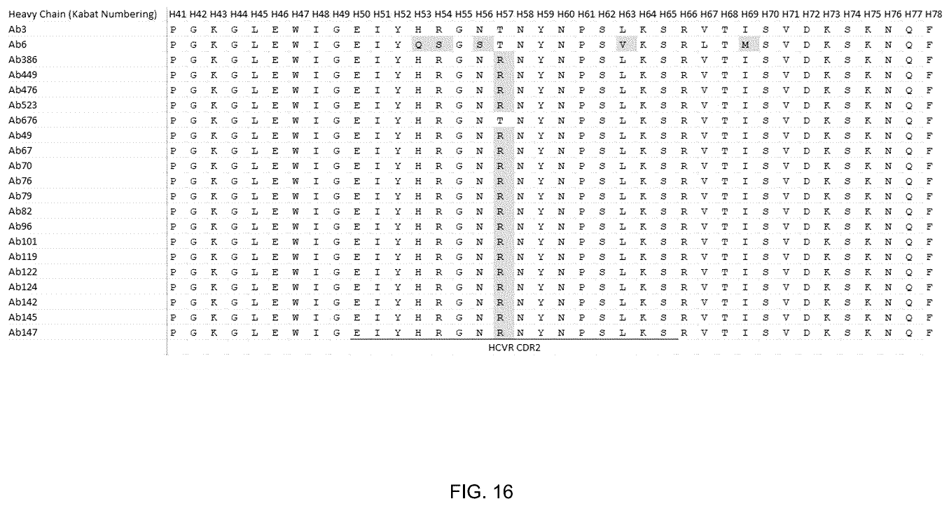

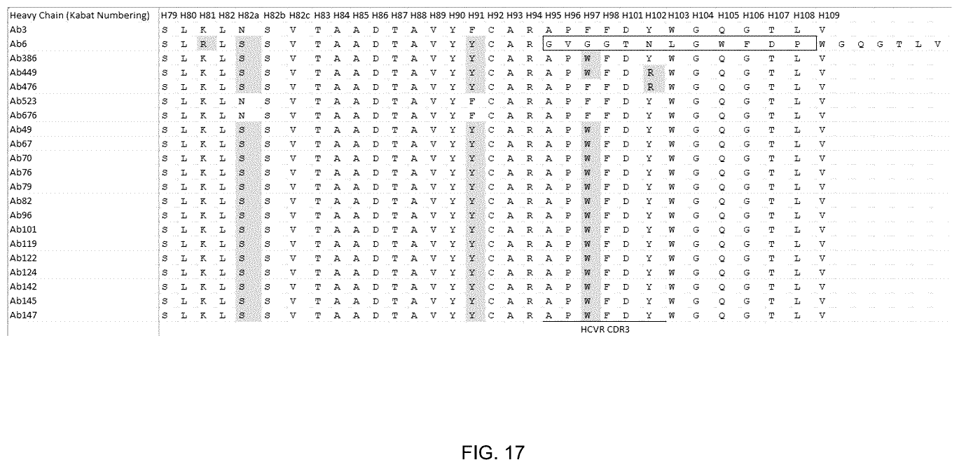

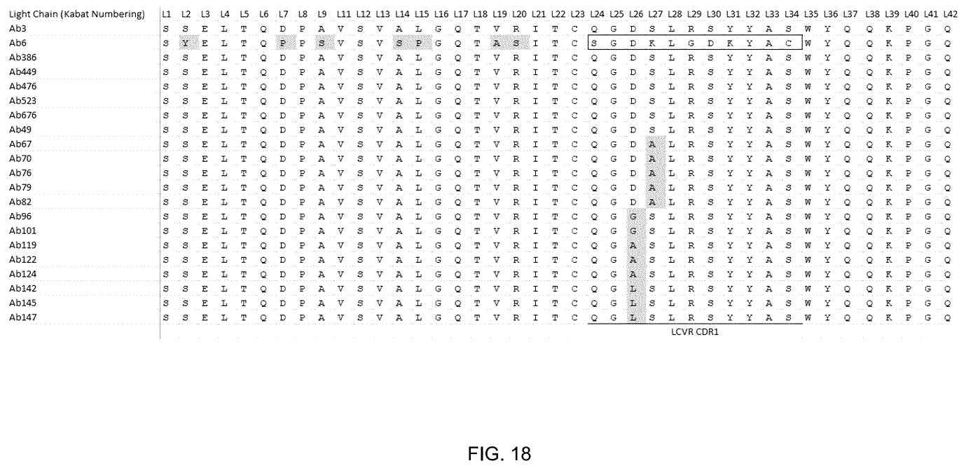

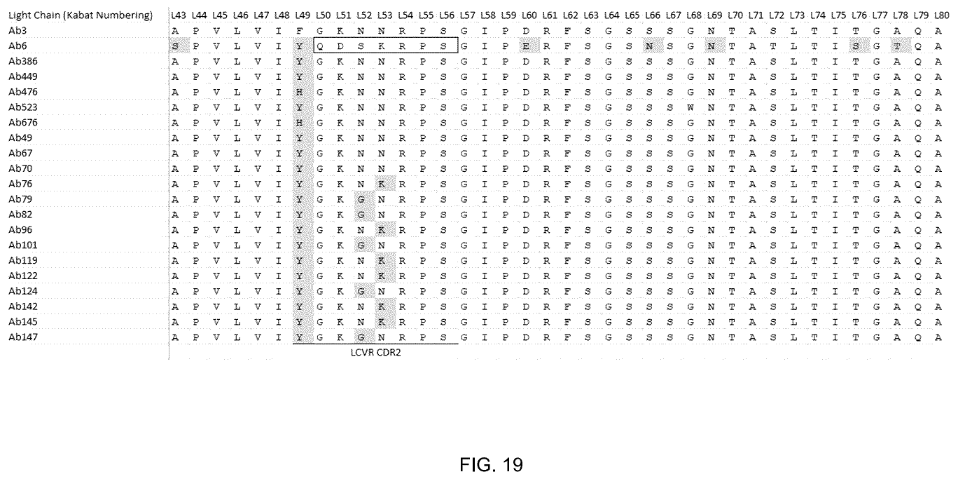

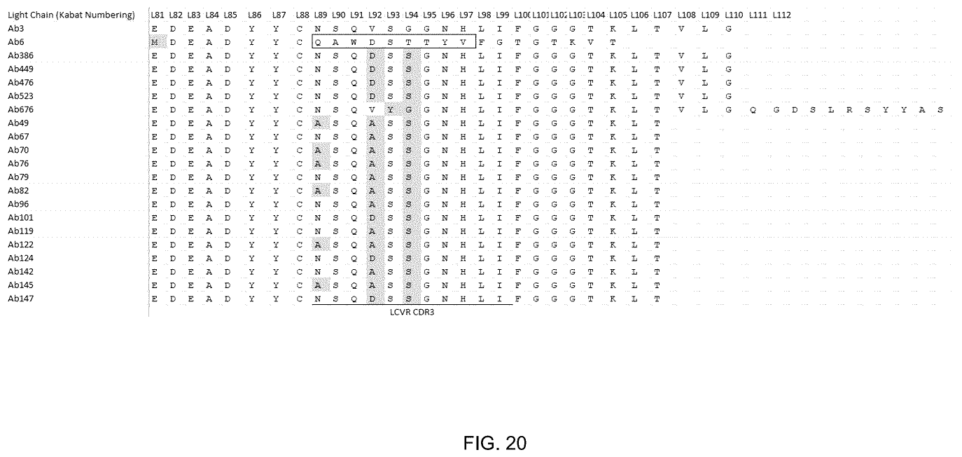

Anti-TrkA antibody variants developed during the development process outlined in the above examples were sequenced, and the sequences aligned. Heavy chain sequences are shown in FIGS. 15-17 comprising HCVR CDR1-3, respectively, and light chain sequences are shown in FIGS. 18-20 comprising LCVR CDR1-3, respectively. These Figures provide visual representations of the modifications, particularly in CDR regions for anti-TrkA antibodies which have similar functionality to Ab82 (see Tables 1-3 above). Shaded residues denote several substitutions that were introduced to the anti-TrkA variants.

### Example 19—Non-Clinical Safety and Efficacy Studies with Ab82

A single dose PK study in monkeys with Ab82 delivered at 4 mg/kg by iv bolus or sc injection or 20 mg/kg by sc injection showed no adverse effects (not shown). Naïve male cynomolgus monkeys were divided into 4 groups. On day 0, monkeys were administered 4 mg/kg Ab82 by iv bolus or sc injection or 20 mg/kg by sc injection (groups 1-3, respectively). An additional 4 monkeys were administered vehicle only (group 4).

No indications of injection site inflammation were observed, and no animals developed clinical signs that could be attributed to a single administration of Ab82 at 4 mg/kg (sc or iv) or at 20 mg/kg (sc). Further, no Ab82-related changes were observed in the hematology parameters evaluated, or in body weight.

The antibody serum concentration time profile of Ab82 in cynomolgus monkeys was characteristic of therapeutic antibodies, with an initial distribution phase and subsequent terminal elimination phase.

### Example 20—Ab82 Effect on Neurite Outgrowth in In-Vitro Diabetic Neuropathy Model, Glucose Toxicity Assay

The effect of Ab82 on neuro-restoration/neurite regeneration in Dorsal Root Ganglion (DRG) neuronal cell line, was tested in ND7/23 cells, in an in vitro DPN model involving glucose insult.

ND7/23 cells were grown in 96 well plates, and on day 2 growth medium was replaced by differentiation medium to allow the cells to differentiate to DRG neurons for a further 5 days. On day 7, the cells were exposed to Ab82 (ranging from 0.1 to 10 nM) or NGF (0.05 nM). After 2 hours, the cells were treated with 200 mM of dissolved D-glucose or with differentiation medium (control) and monitored for neurite outgrowth for the remaining duration of the assay using Incucyte S3. Images were generated every 4 hr, objective×10, 4 sites for each well, using phase channel.

Following incubation for an additional 24 hours, the medium of all cultures was washed and replaced with a fresh differentiation medium containing Ab82 or NGF (same concentrations as in day 7). After an additional 60 hours, the cells were stained with propidium iodide (PI), Calcein-AM Green, and Hoechst, followed by High Content Screen (HCS, ImageXpress Molecular Devices) live imaging (objective×20, 16 sites per well).

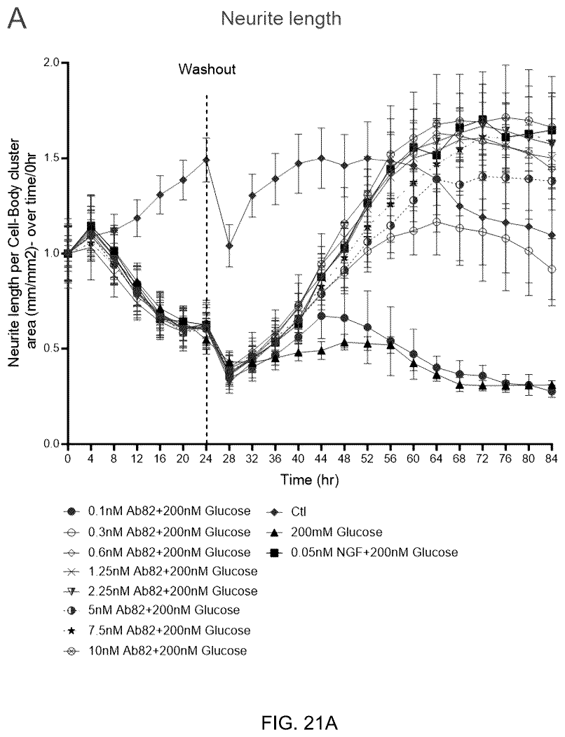

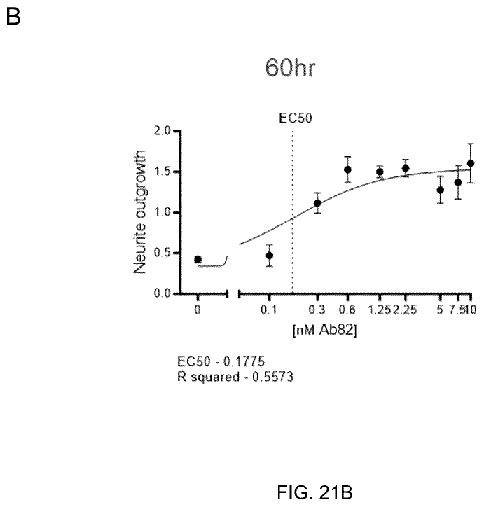

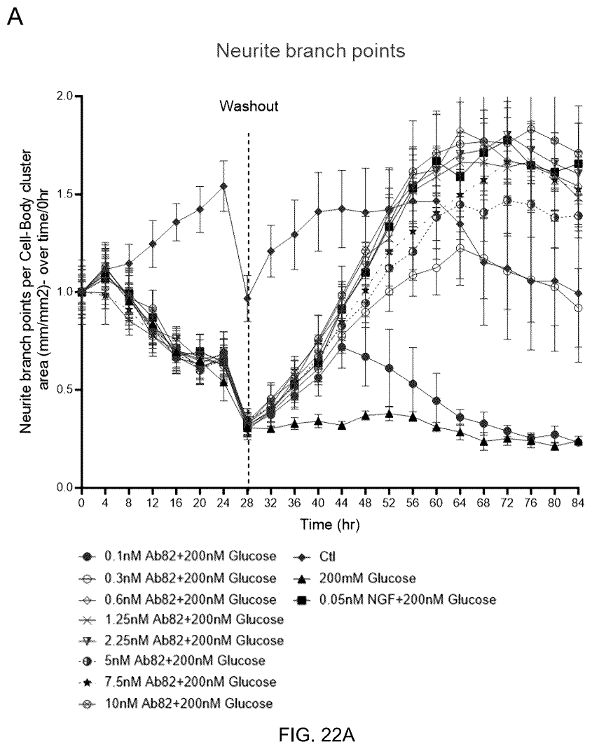

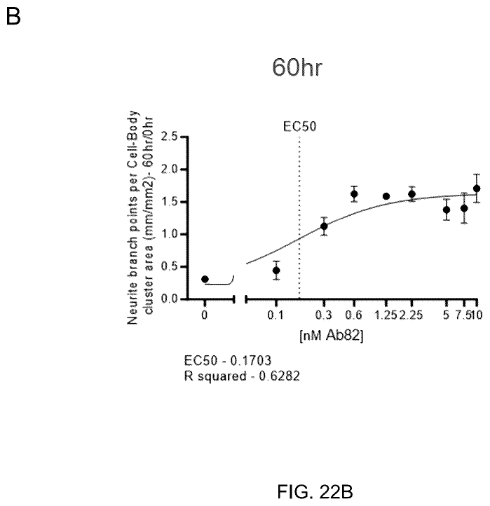

Exposure of ND7/23 cells to 200 mM glucose (glucose insult) for 24 hr led to a robust and rapid collapse of neurites compared to glucose untreated cells (FIGS. 21 and 22). This collapse was not affected by Ab82 nor NGF. Following washout, there was no recovery of neurite outgrowth (neurite length as shown in FIG. 21 and branch points as shown in FIG. 22) in cultures exposed to glucose insult without Ab82 or NGF. However, in cultures exposed to glucose injury and treated with Ab82, a recovery of neurite outgrowth was detected in a dose dependent manner at EC₅₀ of ˜0.2 nM (FIG. 21 B and 22 B). Significant recovery is already evident at a concentration of 0.3 nM of Ab82 (as shown in FIG. 21 B and FIG. 22 B), and EC₉₅ is evident at 0.35 nM. NGF at 0.05 nM had also induced a recovery of neurite outgrowth similar to the maximal effect of Ab82 (as shown in FIG. 21 B and FIG. 22 B). This data shows that Ab82 supports neurites regeneration following glucose insult in a dose dependent manner in ND7/23 DRG neuronal cells.

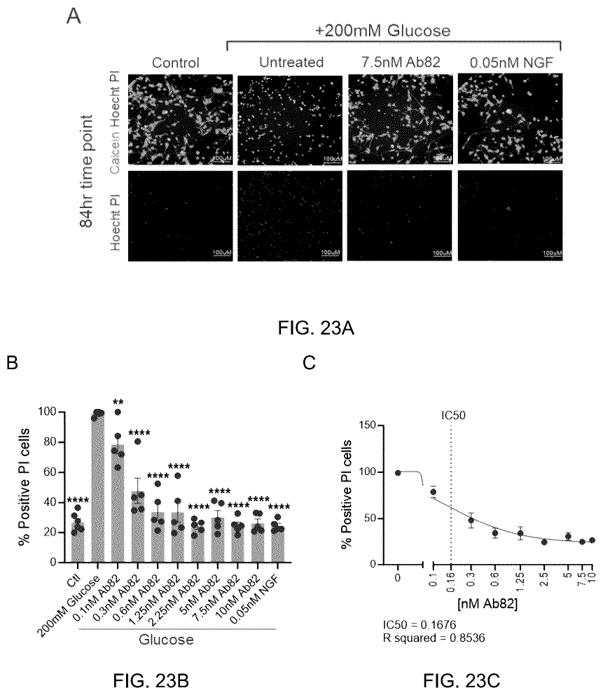

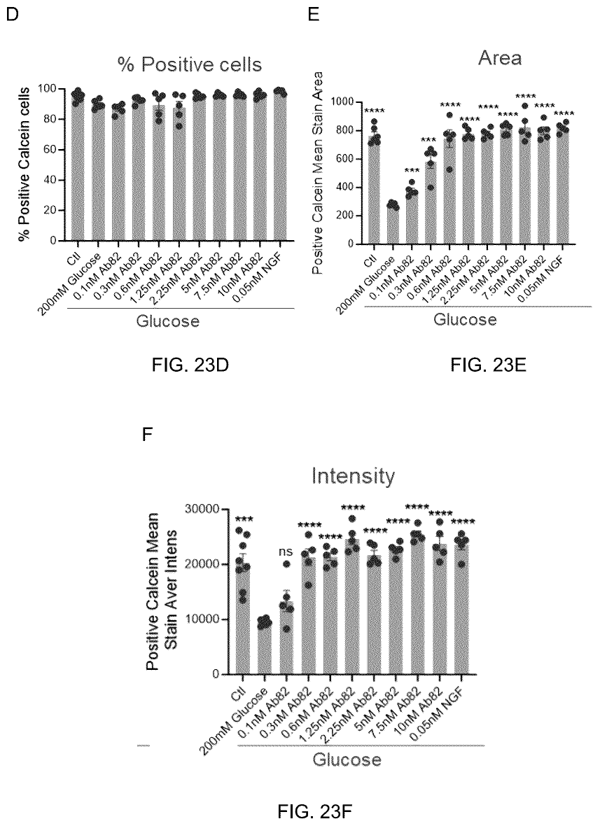

According to propidium iodide staining (PI, membrane impermeable dye), glucose insult induced membrane impairment (% positive PI cells), whereas Ab82 restored the membrane integrity in a dose response manner (FIGS. 23A, B and C). Cell viability was tested using Calcein-AM Green staining. As shown in FIG. 23A, glucose insult at 84 h time point did not affect cell viability (% positive Calcein-AM Green cells), though it reduced cell size (Calcein-AM stained area) and increased cell permeability (reduced Calcein-AM fluorescence intensity). These effects of glucose insult were abolished upon Ab82 treatment in a dose dependent manner (FIG. 23 D-E). This data shows that Ab82 maintains membrane integrity following glucose insult in a dose dependent manner in ND7/23 DRG neuronal cells.

While illustrative embodiments have been illustrated and disclosed, including the best mode known to the inventors for carrying out the antibodies, or antigen-binding fragments thereof, and uses, those skilled in the art will recognize that the disclosure may be practiced with variations on the disclosed antibodies, or antigen-binding fragments thereof, and uses, and such variations are regarded as within the ambit of the disclosure.

Unless specifically stated otherwise, any description as to a possible mechanism or mode of action or reason for improvement is meant to be illustrative only, and the disclosed antibody molecules, methods, and uses are not to be constrained by the correctness or incorrectness of any such suggested mechanism or mode of action or reason for improvement.

Throughout this text, the descriptions refer to antibodies, or antigen-binding fragments thereof, and methods of using said antibodies, or antigen-binding fragments thereof. Where the disclosure describes or claims a feature or embodiment associated with an antibody, or antigen-binding fragment thereof, such a feature or embodiment is equally applicable to the methods of using the antibodies, or antigen-binding fragments thereof. Likewise, where the disclosure describes or claims a feature or embodiment associated with a method of using an antibody, or antigen-binding fragment thereof, such a feature or embodiment is equally applicable to the antibodies, or antigen-binding fragments thereof.

The contents of all references, and published patents and patent applications cited throughout the application are hereby incorporated by reference. Full bibliographic details of references cited herein are collected at the end of the subject specification.

TABLE 7
Sequences

| SEQ ID | | |
|---|---|---|
| No: | Sequence | Description |
| 1 | ASTKGPSVFPLAPSSKSTSGGTAALGCLVKDYFPEPVTVSWNSG | Human IgG1 |
| | ALTSGVHTFPAVLQSS60GLYSLSSVVTVPSSSLGTQTYICNVNH | |
| | KPSNTKVDKKVEPKSCDKTHTCPPCPAPELLGG120PSVFLFPPK | |
| | PKDTLMISRTPEVTCVVVDVSHEDPEVKFNWYVDGVEVHNAKTK | |
| | PREEQYN180STYRVVSVLTVLHQDWLNGKEYKCKVSNKALPAPI | |
| | EKTISKAKGQPREPQVYTLPPSRDE240LTKNQVSLTCLVKGFYP | |
| | SDIAVEWESNGQPENNYKTTPPVLDSDGSFFLYSKLTVDKSRW3 | |
| | 00QQGNVFSCSVMHEALHNHYTQKSLSLSPG | |
| 2 | ASTKGPSVFPLAPSSKSTSGGTAALGCLVKDYFPEPVTVSWNSG | Human IgG1 |
| | ALTSGVHTFPAVLQSSGLYSLSSVVTVPSSSLGTQTYICNVNHKP | FAB TAG |
| | SNTKVDKKVEPKSC | |
| 3 | ASTKGPSVFPLAPSSKSTSGGTAALGCLVKDYFPEPVTVSWNSG | Human IgG1 |
| | ALTSGVHTFPAVLQSSGLYSLSSVVTVPSSSLGTQTYICNVNHKP | KiH Hole |
| | SNTKVDKKVEPKSCDKTHTCPPCPAPELLGGPSVFLFPPKPKDTL | |
| | MISRTPEVTCWVDVSHEDPEVKFNWYVDGVEVHNAKTKPREE | |
| | QYNSTYRVVSVLTVLHQDWLNGKEYKCKVSNKALPAPIEKTISKA | |
| | KGQPREPQVYTLPPSRDELTKNQVSLSCAVKGFYPSDIAVEWES | |
| | NGQPENNYKTTPPVLDSDGSFFLVSKLTVDKSRWQQGNVFSCS | |
| | VMHEALHNHYTQKSLSLSPG | |
| 4 | ASTKGPSVFPLAPSSKSTSGGTAALGCLVKDYFPEPVTVSWNSG | Human IgG1 |
| | ALTSGVHTFPAVLQSSGLYSLSSVVTVPSSSLGTQTYICNVNHKP | KIH Knob |
| | SNTKVDKKVEPKSCDKTHTCPPCPAPELLGGPSVFLFPPKPKDTL | |
| | MISRTPEVTCVVDVSHEDPEVKFNWYVDGVEVHNAKTKPREE | |
| | QYNSTYRVVSVLTVLHQDWLNGKEYKCKVSNKALPAPIEKTISKA | |
| | KGQPREPQVYTLPPSRDELTKNQVSLWCLVKGFYPSDIAVEWES | |
| | NGQPENNYKTTPPVLDSDGSFFLYSKLTVDKSRWQQGNVFSCS | |
| | VMHEALHNHYTQKSLSLSPG | |
| 5 | ASTKGPSVFPLAPSSKSTSGGTAALGCLVKDYFPEPVTVSWNSG | Human IgG1 |
| | ALTSGVHTFPAVLQSSGLYSLSSVVTVPSSSLGTQTYICNVNHKP | (L235A, |
| | SNTKVDKKVEPKSCDKTHTCPPCPAPELAGAPSVFLFPPKPKDTL | G237A) |
| | MISRTPEVTCVVVDVSHEDPEVKFNWYVDGVEVHNAKTKPREE | |
| | QYNSTYRVVSVLTVLHQDWLNGKEYKCKVSNKALPAPIEKTISKA | |
| | KGQPREPQVYTLPPSRDELTKNQVSLTCLVKGFYPSDIAVEWES | |
| | NGQPENNYKTTPPVLDSDGSFFLYSKLTVDKSRWQQGNVFSCS | |
| | VMHEALHNHYTQKSLSLSPG | |
| 6 | ASTKGPSVFPLAPSSKSTSGGTAALGCLVKDYFPEPVTVSWNSG | Human IgG1 |
| | ALTSGVHTFPAVLQSSGLYSLSSVVTVPSSSLGTQTYICNVNHKP | YTE |
| | SNTKVDKKVEPKSCDKTHTCPPCPAPELLGGPSVFLFPPKPKDTL | |
| | YITREPEVTCVVVDVSHEDPEVKFNWYVDGVEVHNAKTKPREEQ | |
| | YNSTYRVVSVLTVLHQDWLNGKEYKCKVSNKALPAPIEKTISKAK | |
| | GQPREPQVYTLPPSRDELTKNQVSLTCLVKGFYPSDIAVEWESN | |
| | GQPENNYKTTPPVLDSDGSFFLYSKLTVDKSRWQQGNVFSCSV | |
| | MHEALHNHYTQKSLSLSPG | |
| 7 | ASTKGPSVFPLAPSSKSTSGGTAALGCLVKDYFPEPVTVSWNSG | Human IgG1- |
| | ALTSGVHTFPAVLQSSGLYSLSSVVTVPSSSLGTQTYICNVNHKP | LALA-PG |
| | SNTKVDKKVEPKSCDKTHTCPPCPAPESTRGPSVFLFPPKPKDT | (L234A/L235A/ |
| | LMISRTPEVTCVVVDVSHEDPEVKFNWYVDGVEVHNAKTKPREE | P329G) |
| | QYNSTYRVVSVLTVLHQDWLNGKEYKCKVSNKALPAPIEKTISKA | |
| | KGQPREPQVYTLPPSRDELTKNQVSLTCLVKGFYPSDIAVEWES | |
| | NGQPENNYKTTPPVLDSDGSFFLYSKLTVDKSRWQQGNVFSCS | |
| | VMHEALHNHYTQKSLSLSPG | |
| 8 | ASTKGPSVFPLAPSSKSTSGGTAALGCLVKDYFPEPVTVSWNSG | Human IgG1- |
| | ALTSGVHTFPAVLQSSGLYSLSSVVTVPSSSLGTQTYICNVNHKP | STR |
| | SNTKVDKKVEPKSCDKTHTCPPCPAPEAAGGPSVFLFPPKPKDT | (L234S/L235T/ |
| | LMISRTPEVTCVVVDVSHEDPEVKFNWYVDGVEVHNAKTKPREE | G236R) |
| | QYNSTYRVVSVLTVLHQDWLNGKEYKCKVSNKALGAPIEKTISKA | |
| | KGQPREPQVYTLPPSRDELTKNQVSLTCLVKGFYPSDIAVEWES | |
| | NGQPENNYKTTPPVLDSDGSFFLYSKLTVDKSRWQQGNVFSCS | |
| | VMHEALHNHYTQKSLSLSPG | |
| 9 | ASTKGPSVFPLAPCSRSTSESTAALGCLVKDYFPEPVTVSWNSG | Human |
| | ALTSGVHTFPAVLQSSGLYSLSSWTVPSSNFGTQTYTCNVDHK | IgG2DASS |
| | PSNTKVDKTVERKCCVECPPCPAPPVAGPSVFLFPPKPKDTLMIS | |
| | RTPEVTCVVVDVSHEDPEVQFNWYVDGVEVHNAKTKPREEQFN | |
| | STFRVVSVLTWVHQDWLNGKEYKCKVSNKGLPSSIEKTISKTKGQ | |
| | PREPQVYTLPPSREEMTKNQVSLTCLVKGFYPSDIAVEWESNGQ | |
| | PENNYKTTPPMLDSDGSFFLYSKLTVDKSRWQQGNVFSCSVMH | |
| | EALHNHYTQKSLSLSPG | |
| 10 | ASTKGPSVFPLAPCSRSTSESTAALGCLVKDYFPEPVTVSWNSG | Human IgG4 |
| | ALTSGVHTFPAVLQSSGLYSLSSVVTVPSSSLGTKTYTCNVDHKP | |
| | SNTKVDKRVESKYGPPCPPCPAPEFLGGPSVFLFPPKPKDTLMIS | |
| | RTPEVTCVVVDVSQEDPEVQFNWYVDGVEVHNAKTKPREEQFN | |
| | STYRVVSVLTVLHQDWLNGKEYKCKVSNKGLPSSIEKTISKAKGQ | |
| | PREPQVYTLPPSQEEMTKNQVSLTCLVKGFYPSDIAVEWESNGQ | |
| | PENNYKTTPPVLDSDGSFFLYSRLTVDKSRWQEGNVFSCSVMH | |
| | EALHNHYTQKSLSLSLG | |
| 11 | ASTKGPSVFPLAPCSRSTSESTAALGCLVKDYFPEPVTVSWNSG | Human IgG4 |
| | ALTSGVHTFPAVLQSSGLYSLSSVVTVPSSSLGTKTYTCNVDHKP | KIH Hole |
| | SNTKVDKRVESKYGPPCPPCPAPEFLGGPSVFLFPPKPKDTLMIS | |
| | RTPEVTCVVVDVSQEDPEVQFNWYVDGVEVHNAKTKPREEQFN | |
| | STYRVVSVLTVLHQDWLNGKEYKCKVSNKGLPSSIEKTISKAKGQ | |
| | PREPQVYTLPPSQEEMTKNQVSLSCAVKGFYPSDIAVEWESNG | |
| | QPENNYKTTPPVLDSDGSFFLVSRLTVDKSRWQEGNVFSCSVM | |
| | HEALHNHYTQKSLSLSLG | |
| 12 | ASTKGPSVFPLAPCSRSTSESTAALGCLVKDYFPEPVTVSWNSG | Human IgG4 |
| | ALTSGVHTFPAVLQSSGLYSLSSVVTVPSSSLGTKTYTCNVDHKP | KIH Knob |
| | SNTKVDKRVESKYGPPCPPCPAPEFLGGPSVFLFPPKPKDTLMIS | |
| | RTPEVTCVVVDVSQEDPEVQFNWYVDGVEVHNAKTKPREEQFN | |
| | STYRVVSVLTVLHQDWLNGKEYKCKVSNKGLPSSIEKTISKAKGQ | |
| | PREPQVYTLPPSQEEMTKNQVSLWCLVKGFYPSDIAVEWESNG | |
| | QPENNYKTTPPVLDSDGSFFLYSRLTVDKSRWQEGNVFSCSVM | |
| | HEALHNHYTQKSLSLSLG | |
| 13 | ASTKGPSVFPLAPCSRSTSESTAALGCLVKDYFPEPVTVSWNSG | Human IgG4 |
| | ALTSGVHTFPAVLQSSGLYSLSSVVTVPSSSLGTKTYTCNVDHKP | (L235A, |
| | SNTKVDKRVESKYGPPCPPCPAPEFAGAPSVFLFPPKPKDTLMIS | G237A) |
| | RTPEVTCVVVDVSQEDPEVQFNWYVDGVEVHNAKTKPREEQFN | |
| | STYRVVSVLTVLHQDWLNGKEYKCKVSNKGLPSSIEKTISKAKGQ | |
| | PREPQVYTLPPSQEEMTKNQVSLTCLVKGFYPSDIAVEWESNGQ | |
| | PENNYKTTPPVLDSDGSFFLYSRLTVDKSRWQEGNVFSCSVMH | |
| | EALHNHYTQKSLSLSLG | |
| 14 | ASTKGPSVFPLAPCSRSTSESTAALGCLVKDYFPEPVTVSWNSG | Human IgG4 |
| | ALTSGVHTFPAVLQSSGLYSLSSVVTVPSSSLGTKTYTCNVDHKP | (L235E) |
| | SNTKVDKRVESKYGPPCPPCPAPEFEGGPSVFLFPPKPKDTLMI | |
| | SRTPEVTCVVVDVSQEDPEVQFNWYVDGVEVHNAKTKPREEQF | |
| | NSTYRVVSVLTVLHQDWLNGKEYKCKVSNKGLPSSIEKTISKAKG | |
| | QPREPQVYTLPPSQEEMTKNQVSLTCLVKGFYPSDIAVEWESNG | |
| | QPENNYKTTPPVLDSDGSFFLYSRLTVDKSRWQEGNVFSCSVM | |
| | HEALHNHYTQKSLSLSLG | |
| 15 | ASTKGPSVFPLAPCSRSTSESTAALGCLVKDYFPEPVTVSWNSG | Human IgG4 |
| | ALTSGVHTFPAVLQSSGLYSLSSVVTVPSSSLGTKTYTCNVDHKP | YTE |
| | SNTKVDKRVESKYGPPCPPCPAPEFLGGPSVFLFPPKPKDTLYIT | |
| | REPEVTCVVVDVSQEDPEVQFNWYVDGVEVHNAKTKPREEQFN | |
| | STYRVVSVLTVLHQDWLNGKEYKCKVSNKGLPSSIEKTISKAKGQ | |
| | PREPQVYTLPPSQEEMTKNQVSLTCLVKGFYPSDIAVEWESNGQ | |
| | PENNYKTTPPVLDSDGSFFLYSRLTVDKSRWQEGNVFSCSVMH | |
| | EALHNHYTQKSLSLSLG | |
| 16 | ASTKGPSVFPLAPCSRSTSESTAALGCLVKDYFPEPVTVSWNSG | Human IgG4 |
| | ALTSGVHTFPAVLQSSGLYSLSSVVTVPSSSLGTKTYTCNVDHKP | YTE KIH Hole |
| | SNTKVDKRVESKYGPPCPPCPAPEFLGGPSVFLFPPKPKDTLYIT | |
| | REPEVTCVVVDVSQEDPEVQFNWYVDGVEVHNAKTKPREEQFN | |
| | STYRVVSVLTVLHQDWLNGKEYKCKVSNKGLPSSIEKTISKAKGQ | |
| | PREPQVYTLPPSQEEMTKNQVSLSCAVKGFYPSDIAVEWESNG | |
| | QPENNYKTTPPVLDSDGSFFLVSRLTVDKSRWQEGNVFSCSVM | |
| | HEALHNHYTQKSLSLSLG | |
| 17 | ASTKGPSVFPLAPCSRSTSESTAALGCLVKDYFPEPVTVSWNSG | Human IgG4 |
| | ALTSGVHTFPAVLQSSGLYSLSSVVTVPSSSLGTKTYTCNVDHKP | YTE KIH Knob |
| | SNTKVDKRVESKYGPPCPPCPAPEFLGGPSVFLFPPKPKDTLYIT | |
| | REPEVTCVVVDVSQEDPEVQFNWYVDGVEVHNAKTKPREEQFN | |
| | STYRVVSVLTVLHQDWLNGKEYKCKVSNKGLPSSIEKTISKAKGQ | |
| | PREPQVYTLPPSQEEMTKNQVSLWCLVKGFYPSDIAVEWESNG | |
| | QPENNYKTTPPVLDSDGSFFLYSRLTVDKSRWQEGNVFSCSVM | |
| | HEALHNHYTQKSLSLSLG | |
| 18 | ASTKGPSVFPLAPSSKSTSGGTAALGCLVKDYFPEPVTVSWNSG | Human IgG1 |
| | ALTSGVHTFPAVLQSSGLYSLSSVVTVPSSSLGTQTYICNVNHKP | (L235A, |
| | SNTKVDKKVEPKSCDKTHTCPPCPAPELAGAPSVFLFPPKPKDTL | G237A), YTE |
| | YITREPEVTCVVVDVSHEDPEVKFNWYVDGVEVHNAKTKPREEQ | |
| | YNSTYRVVSVLTVLHQDWLNGKEYKCKVSNKALPAPIEKTISKAK | |
| | GQPREPQVYTLPPSRDELTKNQVSLTCLVKGFYPSDIAVEWESN | |
| | GQPENNYKTTPPVLDSDGSFFLYSKLTVDKSRWQQGNVFSCSV | |
| | MHEALHNHYTQKSLSLSPG | |
| 19 | RTVAAPSVFIFPPSDEQLKSGTASVVCLLNNFYPREAKVQWKVD | Human kappa, |
| | NALQSGNSQESVTEQDSKDSTYSLSSTLTLSKADYEKHKVYACE | light chain |
| | VTHQGLSSPVTKSFNRGEC | constant |
| | | region |
| | | sequence |
| 20 | GQPKAAPSVTLFPPSSEELQANKATLVCLISDFYPGAVTVAWKAD | Human |
| | SSPVKAGVETTTPSKQSNNKYAASSYLSLTPEQWKSHRSYSCQV | lambda, light |
| | THEGSTVEKTVAPTECS | chain constant |
| | | region |
| | | sequence |
| 21 | GGSXSSXNWW T | Heavy chain |
| | | variable region |
| | | (HCVR) CDR1 |
| | | consensus |
| 22 | EIYHRGNXNY NPSLKS | Heavy chain |
| | | variable region |
| | | (HCVR) CDR2 |
| | | consensus |
| 23 | APXFDX | Heavy chain |
| | | variable region |
| | | (HCVR) CDR3 |
| | | consensus |
| 24 | QGXXLRSYYA S | Heavy chain |
| | | variable region |
| | | (LCVR) CDR1 |
| | | consensus |
| 25 | GKXXRPS | Heavy chain |
| | | variable region |
| | | (LCVR) CDR2 |
| | | consensus |
| 26 | XSQXXXGNHL I | Heavy chain |
| | | variable region |
| | | (LCVR) CDR3 |
| | | consensus |
| 27 | QVQLQESGPGLVKPSGTLSLTCAVSGGSVSSGNWWTWVRQPP | Heavy chain |
| | GKGLEWIGEIYHRGNTNYNPSLKSRVTISVDKSKNQFSLKLNSVT | variable region |
| | AADTAVYFCARAPFFDYWGQGTLV | (HCVR) Ab 3 |
| 28 | SSELTQDPAVSVALGQTVRITCQGDSLRSYYASWYQQKPGQAP | Light chain |
| | VLVIFGKNNRPSGIPDR | variable region |
| | FSGSSSGNTASLTITGAQAEDEADYYCNSQVSGGNHLIFGGGTK | (HCVR) Ab 3 |
| | LTVLG | |
| 29 | QVQLQESGPGLVKPSGTLSLTCAVSGGSISSSNWWTWVRQPPG | Heavy chain |
| | KGLEWIGEIYHRGNRNYNPSLKSRVTISVDKSKNQFSLKLSSVTA | variable region |
| | ADTAVYYCARAPWFDYWGQGTLV | (HCVR) Ab |
| | | 386 |
| 30 | SSELTQDPAVSVALGQTVRITCQGDSLRSYYASWYQQKPGQAP | Light chain |
| | VLVIYGKNNRPSGIPDRFSGSSSGNTASLTITGAQAEDEADYYCN | variable region |
| | SQDSSGNHLIFGGGTKLTVLG | (HCVR) Ab |
| | | 386 |
| 31 | QVQLQESGPGLVKPSGTLSLTCAVSGGSISSSNWWTWVRQPPG | Heavy chain |
| | KGLEWIGEIYHRGNRNYNPSLKSRVTISVDKSKNQFSLKLSSVTA | variable region |
| | ADTAVYYCARAPWFDRWGQGTLV | (HCVR) Ab |
| | | 449 |
| 32 | SSELTQDPAVSVALGQTVRITCQGDSLRSYYASWYQQKPGQAP | Light chain |
| | VLVIYGKNNRPSGIPDRFSGSSSGNTASLTITGAQAEDEADYYCN | variable region |
| | SQDSSGNHLIFGGGTKLTVLG | (HCVR) Ab |
| | | 449 |
| 33 | QVQLQESGPGLVKPSGTLSLTCAVSGGSISSSNWWTWVRQPPG | Heavy chain |
| | KGLEWIGEIYHRGNRNYNPSLKSRVTISVDKSKNQFSLKLSSVTA | variable region |
| | ADTAVYYCARAPFFDRWGQGTLV | (HCVR) Ab |
| | | 476 |
| 34 | SSELTQDPAVSVALGQTVRITCQGDSLRSYYASWYQQKPGQAP | Light chain |
| | VLVIHGKNNRPSGIPDRFSGSSSGNTASLTITGAQAEDEADYYCN | variable region |
| | SQDSSGNHLIFGGGTKLTVLG | (HCVR) Ab |
| | | 476 |
| 35 | QVQLQESGPGLVKPSGTLSLTCAVSGGSVSSGNWWTWVRQPP | Heavy chain |
| | GKGLEWIGEIYHRGNRNYNPSLKSRVTISVDKSKNQFSLKLNSVT | variable region |
| | AADTAVYFCARAPFFDYWGQGTLV | (HCVR) Ab |
| | | 523 |
| 36 | SSELTQDPAVSVALGQTVRITCQGDSLRSYYASWYQQKPGQAP | Light chain |
| | VLVIYGKNNRPSGIPDRFSGSSSWNTASLTITGAQAEDEADYYCN | variable region |
| | SQDSSGNHLIFGGGTKLTVLG | (HCVR) Ab |
| | | 523 |
| 37 | QVQLQESGPGLVKPSGTLSLTCAVSGGSVSSGNWWTWVRQPP | Heavy chain |
| | GKGLEWIGEIYHRGNTNYNPSLKSRVTISVDKSKNQFSLKLNSVT | variable region |
| | AADTAVYFCARAPFFDYWGQGTLV | (HCVR) Ab |
| | | 676 |
| 38 | SSELTQDPAVSVALGQTVRITCQGDSLRSYYASWYQQKPGQAP | Light chain |
| | VLVIHGKNNRPSGIPDRFSGSSSGNTASLTITGAQAEDEADYYCN | variable region |
| | SQVYGGNHLIFGGGTKLTVLGQGDSLRSYYAS | (HCVR) Ab |
| | | 676 |
| 39 | EVQLQESGPGLVKPSGTLSLTCAVSGGSISSSNWWTWVRQPPG | Heavy chain |
| | KGLEWIGEIYHRGNRNYNPSLKSRVTISVDKSKNQFSLKLSSVTA | variable region |
| | ADTAVYYCARAPWFDYWGQGTLV | (HCVR) Ab 49 |
| 40 | SSELTQDPAVSVALGQTVRITCQGDSLRSYYASWYQQKPGQAP | Light chain |
| | VLVIYGKNNRPSGIPDRFSGSSSGNTASLTITGAQAEDEADYYCA | variable region |
| | SQASSGNHLIFGGGTKLT | (HCVR) Ab 49 |
| 41 | EVQLQESGPGLVKPSGTLSLTCAVSGGSISSSNWWTWVRQPPG | Heavy chain |
| | KGLEWIGEIYHRGNRNYNPSLKSRVTISVDKSKNQFSLKLSSVTA | variable region |
| | ADTAVYYCARAPWFDYWGQGTLV | (HCVR) Ab 67 |
| 42 | SSELTQDPAVSVALGQTVRITCQGDALRSYYASWYQQKPGQAP | Light chain |
| | VLVIYGKNNRPSGIPDRFSGSSSGNTASLTITGAQAEDEADYYCN | variable region |
| | SQASSGNHLIFGGGTKLT | (HCVR) Ab 67 |
| 43 | EVQLQESGPGLVKPSGTLSLTCAVSGGSISSSNWWTWVRQPPG | Heavy chain |
| | KGLEWIGEIYHRGNRNYNPSLKSRVTISVDKSKNQFSLKLSSVTA | variable region |
| | ADTAVYYCARAPWFDYWGQGTLV | (HCVR) Ab 70 |
| 44 | SSELTQDPAVSVALGQTVRITCQGDALRSYYASWYQQKPGQAP | Light chain |
| | VLVIYGKNNRPSGIPDRFSGSSSGNTASLTITGAQAEDEADYYCA | variable region |
| | SQASSGNHLIFGGGTKLT | (HCVR) Ab 70 |
| 45 | EVQLQESGPGLVKPSGTLSLTCAVSGGSISSSNWWTWVRQPPG | Heavy chain |
| | KGLEWIGEIYHRGNRNYNPSLKSRVTISVDKSKNQFSLKLSSVTA | variable region |
| | ADTAVYYCARAPWFDYWGQGTLV | (HCVR) Ab 76 |
| 46 | SSELTQDPAVSVALGQTVRITCQGDALRSYYASWYQQKPGQAP | Light chain |
| | VLVIYGKNKRPSGIPDRFSGSSSGNTASLTITGAQAEDEADYYCA | variable region |
| | SQASSGNHLIFGGGTKLT | (HCVR) Ab 76 |
| 47 | EVQLQESGPGLVKPSGTLSLTCAVSGGSISSSNWWTWVRQPPG | Heavy chain |
| | KGLEWIGEIYHRGNRNYNPSLKSRVTISVDKSKNQFSLKLSSVTA | variable region |
| | ADTAVYYCARAPWFDYWGQGTLV | (HCVR) Ab 79 |
| 48 | SSELTQDPAVSVALGQTVRITCQGDALRSYYASWYQQKPGQAP | Light chain |
| | VLVIYGKGNRPSGIPDRFSGSSSGNTASLTITGAQAEDEADYYCN | variable region |
| | SQASSGNHLIFGGGTKLT | (HCVR) Ab 79 |
| 49 | QVQLQESGPGLVKPSGTLSLTCAVSGGSISSSNWWTWVRQPPG | Heavy chain |
| | KGLEWIGEIYHRGNRNYNPSLKSRVTISVDKSKNQFSLKLSSVTA | variable region |
| | ADTAVYYCARAPWFDYWGQGTLV | (HCVR) Ab 82 |
| 50 | SSELTQDPAVSVALGQTVRITCQGDALRSYYASWYQQKPGQAP | Light chain |
| | VLVIYGKGNRPSGIPDRFSGSSSGNTASLTITGAQAEDEADYYCA | variable region |
| | SQASSGNHLIFGGGTKLT | (HCVR) Ab 82 |
| 51 | EVQLQESGPGLVKPSGTLSLTCAVSGGSISSSNWWTWVRQPPG | Heavy chain |
| | KGLEWIGEIYHRGNRNYNPSLKSRVTISVDKSKNQFSLKLSSVTA | variable region |
| | ADTAVYYCARAPWFDYWGQGTLV | (HCVR) Ab 96 |
| 52 | SSELTQDPAVSVALGQTVRITCQGGSLRSYYASWYQQKPGQAP | Light chain |
| | VLVIYGKNKRPSGIPDRFSGSSSGNTASLTITGAQAEDEADYYCN | variable region |
| | SQASSGNHLIFGGGTKLT | (HCVR) Ab 96 |
| 53 | EVQLQESGPGLVKPSGTLSLTCAVSGGSISSSNWWTWVRQPPG | Heavy chain |
| | KGLEWIGEIYHRGNRNYNPSLKSRVTISVDKSKNQFSLKLSSVTA | variable region |
| | ADTAVYYCARAPWFDYWGQGTLV | (HCVR) Ab |
| | | 101 |
| 54 | SSELTQDPAVSVALGQTVRITCQGGSLRSYYASWYQQKPGQAP | Light chain |
| | VLVIYGKGNRPSGIPDRFSGSSSGNTASLTITGAQAEDEADYYCN | variable region |
| | SQDSSGNHLIFGGGTKLT | (HCVR) Ab |
| | | 101 |
| 55 | EVQLQESGPGLVKPSGTLSLTCAVSGGSISSSNWWTWVRQPPG | Heavy chain |
| | KGLEWIGEIYHRGNRNYNPSLKSRVTISVDKSKNQFSLKLSSVTA | variable region |
| | ADTAVYYCARAPWFDYWGQGTLV | (HCVR) Ab |
| | | 119 |
| 56 | SSELTQDPAVSVALGQTVRITCQGASLRSYYASWYQQKPGQAP | Light chain |
| | VLVIYGKNKRPSGIPDRFSGSSSGNTASLTITGAQAEDEADYYCN | variable region |
| | SQASSGNHLIFGGGTKLT | (HCVR) Ab |
| | | 119 |
| 57 | EVQLQESGPGLVKPSGTLSLTCAVSGGSISSSNWWTWVRQPPG | Heavy chain |
| | KGLEWIGEIYHRGNRNYNPSLKSRVTISVDKSKNQFSLKLSSVTA | variable region |
| | ADTAVYYCARAPWFDYWGQGTLV | (HCVR) Ab |
| | | 122 |
| 58 | SSELTQDPAVSVALGQTVRITCQGASLRSYYASWYQQKPGQAP | Light chain |
| | VLVIYGKNKRPSGIPDRFSGSSSGNTASLTITGAQAEDEADYYCA | variable region |
| | SQASSGNHLIFGGGTKLT | (HCVR) Ab |
| | | 122 |
| 59 | EVQLQESGPGLVKPSGTLSLTCAVSGGSISSSNWWTWVRQPPG | Heavy chain |
| | KGLEWIGEIYHRGNRNYNPSLKSRVTISVDKSKNQFSLKLSSVTA | variable region |
| | ADTAVYYCARAPWFDYWGQGTLV | (HCVR) Ab |
| | | 124 |
| 60 | SSELTQDPAVSVALGQTVRITCQGASLRSYYASWYQQKPGQAP | Light chain |
| | VLVIYGKGNRPSGIPDRFSGSSSGNTASLTITGAQAEDEADYYCN | variable region |
| | SQDSSGNHLIFGGGTKLT | (HCVR) Ab |
| | | 124 |
| 61 | EVQLQESGPGLVKPSGTLSLTCAVSGGSISSSNWWTWVRQPPG | Heavy chain |
| | KGLEWIGEIYHRGNRNYNPSLKSRVTISVDKSKNQFSLKLSSVTA | variable region |
| | ADTAVYYCARAPWFDYWGQGTLV | (HCVR) Ab |
| | | 142 |
| 62 | SSELTQDPAVSVALGQTVRITCQGLSLRSYYASWYQQKPGQAPV | Light chain |
| | LVIYGKNKRPSGIPDRFSGSSSGNTASLTITGAQAEDEADYYCNS | variable region |
| | QASSGNHLIFGGGTKLT | (HCVR) Ab |
| | | 142 |
| 63 | EVQLQESGPGLVKPSGTLSLTCAVSGGSISSSNWWTWVRQPPG | Heavy chain |
| | KGLEWIGEIYHRGNRNYNPSLKSRVTISVDKSKNQFSLKLSSVTA | variable region |
| | ADTAVYYCARAPWFDYWGQGTLV | (HCVR) Ab |
| | | 145 |
| 64 | SSELTQDPAVSVALGQTVRITCQGLSLRSYYASWYQQKPGQAPV | Light chain |
| | LVIYGKNKRPSGIPDRFSGSSSGNTASLTITGAQAEDEADYYCAS | variable region |
| | QASSGNHLIFGGGTKLT | (HCVR) Ab |
| | | 145 |
| 65 | EVQLQESGPGLVKPSGTLSLTCAVSGGSISSSNWWTWVRQPPG | Heavy chain |
| | KGLEWIGEIYHRGNRNYNPSLKSRVTISVDKSKNQFSLKLSSVTA | variable region |
| | ADTAVYYCARAPWFDYWGQGTLV | (HCVR) Ab |
| | | 147 |
| 66 | SSELTQDPAVSVALGQTVRITCQGLSLRSYYASWYQQKPGQAPV | Light chain |
| | LVIYGKGNRPSGIPDRFSGSSSGNTASLTITGAQAEDEADYYCNS | variable region |
| | QDSSGNHLIFGGGTKLT | (HCVR) Ab |
| | | 147 |
| 67 | QVQLQESGPGLVKPSGTLSLTCAVSGGSISSSNWWSWVRQPPG | IGHV4-4*02 |
| | KGLEWIGEIYHSGSTNYNPSLKSRVTISVDKSKNQFSLKLSSVTAA | heavy chain |
| | DTAVYYCAR | |
| 68 | SSELTQDPAVSVALGQTVRITCQGDSLRSYYASWYQQKPGQAP | IGLV3-19*01 |
| | VLVIYGKNNRPSGIPDRFSGSSSGNTASLTITGAQAEDEADYYCN | light chain |
| | SRDSSGNH | |
| 69 | QVQLQESGPGLVKPSGTLSLTCAVSGGSISSSNWWTWVRQPPG | GL3 variant |
| | KGLEWIGEIYHRGNTNYNPSLKSRVTISVDKSKNQFSLKLSSVTA | heavy chain |
| | ADTAVYYCARAPFFDYWGQGTLV | |
| 70 | SSELTQDPAVSVALGQTVRITCQGDSLRSYYASWYQQKPGQAP | GL3 variant |
| | VLVIYGKNNRPSGIPDRFSGSSSGNTASLTITGAQAEDEADYYCN | light chain |
| | SQDSSGNHLIFGGGTKLTVLG | |
| 71 | GGSISSSNWWT | Ab82 HCDR1 |
| 72 | EIYHRGNRNYNPSLKS | Ab82 HCDR2 |
| 73 | APWFDY | Ab82 HCDR3 |
| 74 | QGDALRSYYAS | Ab82 LCDR1 |
| 75 | GKGNRPS | Ab82 LCDR2 |
| 76 | ASQASSGNHLI | Ab82 LCDR3 |
| 77 | QVQLQESGPGLVKPSGTLSLTCAVSGGSISSSNWWTWVRQPPG | Ab82 full |
| | KGLEWIGEIYHRGNRNYNPSLKSRVTISVDKSKNQFSLKLSSVTA | length heavy |
| | ADTAVYYCARAPWFDYWGQGTLVTVSSASTKGPSVFPLAPSSK | chain |
| | STSGGTAALGCLVKDYFPEPVTVSWNSGALTSGVHTFPAVLQSS | |
| | GLYSLSSVVTVPSSSLGTQTYICNVNHKPSNTKVDKKVEPKSCDK | |
| | THTCPPCPAPELAGAPSVFLFPPKPKDTLMISRTPEVTCVVVDVS | |
| | HEDPEVKFNWYVDGVEVHNAKTKPREEQYNSTYRVVSVLTVLH | |
| | QDWLNGKEYKCKVSNKALPAPIEKTISKAKGQPREPQVYTLPPS | |
| | RDELTKNQVSLTCLVKGFYPSDIAVEWESNGQPENNYKTTPPVL | |
| | DSDGSFFLYSKLTVDKSRWQQGNVFSCSVMHEALHNHYTQKSL | |
| | SLSPG | |
| 78 | SSELTQDPAVSVALGQTVRITCQGDALRSYYASWYQQKPGQAP | Ab82 full |
| | VLVIYGKGNRPSGIPDRFSGSSSGNTASLTITGAQAEDEADYYCA | length light |
| | SQASSGNHLIFGGGTKLTVLGQPKAAPSVTLFPPSSEELQANKAT | Chain |
| | LVCLISDFYPGAVTVAWKADSSPVKAGVETTTPSKQSNNKYAAS | |
| | SYLSLTPEQWKSHRSYSCQVTHEGSTVEKTVAPTECS | |
| 79 | QVQLQESGPGLVKPSGTLSLTCAVSGGSISSSNWWTWVRQPPG | Ab82 F(ab')2 |
| | KGLEWIGEIYHRGNRNYNPSLKSRVTISVDKSKNQFSLKLSSVTA | lgG1 |
| | ADTAVYYCARAPWFDYWGQGTLVTVSSASTKGPSVFPLAPSSK | |
| | STSGGTAALGCLVKDYFPEPVTVSWNSGALTSGVHTFPAVLQSS | |
| | GLYSLSSVVTVPSSSLGTQTYICNVNHKPSNTKVDKKVEPKSCDK | |
| | THTCPPCPAPEL | |

What is claimed is:

1. An anti-tropomyosin receptor kinase A (TrkA) antibody, or an antigen-binding fragment thereof, comprising:
a heavy chain complementarity determining region 1 (HCDR1) comprising the amino acid sequence of SEQ ID NO: 71, a HCDR2 comprising the amino acid sequence of SEQ ID NO: 72, a HCDR3 comprising the amino acid sequence of SEQ ID NO: 73, a light chain complementarity determining region 1 (LCDR1) comprising the amino acid sequence of SEQ ID NO: 74, a LCDR2 comprising the amino acid sequence of SEQ ID NO: 75, and a LCDR3 comprising the amino acid sequence of SEQ ID NO: 76.

2. The anti-TrkA antibody, or antigen-binding fragment thereof, of claim 1, comprising a heavy chain variable region (VH) comprising the amino acid sequence of SEQ ID NO: 49 and a light chain variable region (VL) comprising the amino acid sequence of SEQ ID NO: 50.

3. The anti-TrkA antibody, or antigen-binding fragment thereof, of claim 1, wherein the antibody is a monoclonal antibody.

4. The anti-TrkA antibody, or antigen-binding fragment thereof, of claim 1, wherein the antibody is a human antibody.

5. The anti-TrkA antibody, or antigen-binding fragment thereof, of claim 1, wherein the antigen-binding fragment is a F(ab′)2.

6. The anti-TrkA antibody, or antigen-binding fragment thereof, of claim 1, wherein the antigen-binding fragment is not a Fab.

7. The anti-TrkA antibody, or antigen-binding fragment thereof, of claim 1, comprising an IgG1 heavy chain constant region, an IgG2 heavy chain constant region, or an IgG4 heavy chain constant region.

8. The anti-TrkA antibody, or antigen-binding fragment thereof, of claim 7, comprising an IgG1 heavy chain constant region that comprises an L235A substitution and a G237A substitution.

9. The anti-TrkA antibody, or antigen-binding fragment thereof, of claim 1, comprising a heavy chain (HC) comprising the amino acid sequence of SEQ ID NO: 77 and a light chain (LC) comprising the amino acid sequence of SEQ ID NO: 78.

10. A composition comprising the anti-TrkA antibody, or antigen-binding fragment thereof, of claim 1 and a pharmaceutically acceptable carrier.

11. A nucleic acid molecule encoding the anti-TrkA antibody, or antigen-binding fragment thereof, of claim 1.

12. An expression vector comprising the nucleic acid molecule of claim 11.

13. A host cell comprising the nucleic acid molecule according to claim 11.

14. A host cell comprising the expression vector of claim 12.

15. A method of promoting neurite regeneration in a subject in need thereof or treating a neuronal dysfunction in a subject in need thereof, the method comprising:
administering a therapeutically effective amount of the composition of claim 10 to the subject to thereby promote the neurite regeneration or treat the neuronal dysfunction.

16. The method of claim 15, wherein the neuronal dysfunction is:
a diabetic peripheral neuropathy,
a chemotherapy-induced peripheral neuropathy,
a small fiber neuropathy,
a nerve injury,
a wound healing, or
an optic neuropathy.

17. The method of claim 16, wherein the nerve injury is a traumatic nerve injury or a post-surgical nerve injury.

18. The method of claim 16, wherein the wound healing is healing of diabetic foot ulcers.

19. The method of claim 16, wherein the optic neuropathy is glaucoma.

20. An anti-tropomyosin receptor kinase A (TrkA) antibody, or antigen-binding fragment thereof, comprising:
(a) a HCDR1 comprising the amino acid sequence of SEQ ID NO: 21,
(b) a HCDR2 comprising the amino acid sequence of SEQ ID NO: 22,
(c) a HCDR3 comprising the amino acid sequence of SEQ ID NO: 23;
(d) a LCDR1 comprising the amino acid sequence of SEQ ID NO: 24,
(e) a LCDR2 comprising the amino acid sequence of SEQ ID NO: 25, and
(f) a LCDR3 comprising the amino acid sequence of SEQ ID NO: 26,
wherein
the Xb residue in SEQ ID NO: 21 is V or I,
the Xc residue in SEQ ID NO: 21 is G or S,
the Xd residue in SEQ ID NO: 22 is T or R,
the Xg residue in SEQ ID NO: 23 is F or W,
the Xh residue in SEQ ID NO: 23 is R or Y,
the X1 residue in SEQ ID NO: 24 is D, G, A or L,
the X2 residue in SEQ ID NO: 24 is S or A,
the X4 residue in SEQ ID NO: 25 is N or G,
the X5 residue in SEQ ID NO: 25 is N or K,
the X6 residue in SEQ ID NO: 26 is N or A,
the X7 residue in SEQ ID NO: 26 is V, D or A,
the X8 residue in SEQ ID NO: 26 is S or Y, and
the X9 residue in SEQ ID NO: 26 is G or S
and wherein the antibody or antigen-binding fragment thereof selectively binds to and activates TrkA and has
(i) a dissociation constant (KD) for binding human TrkA ranging from about 0.1 nM to about 1,000 nM, preferably about 0.1 nM to about 10 nM, as determined by surface plasmon resonance; and/or
(ii) a half-maximal effective concentration (EC₅₀) value for binding human TrkA ranging from about 10 nM to about 1,000 nM, preferably about 100 nM to about 500 nM.

21. The anti-TrkA antibody, or antigen-binding fragment thereof, of claim 20, wherein the dissociation constant (KD) for binding human TrkA ranges from about 0.1 nM to about 10 nM.

22. The anti-TrkA antibody, or antigen-binding fragment thereof, of claim 20, wherein the half-maximal effective concentration (EC₅₀) value for binding human TrkA ranges from about 100 nM to about 500 nM.

23. The anti-TrkA antibody, or antigen-binding fragment thereof, of claim 20, wherein:
the Xb residue in SEQ ID NO: 21 is I,
the Xc residue in SEQ ID NO: 21 is S,
the Xd residue in SEQ ID NO: 22 is R,
the Xg residue in SEQ ID NO: 23 is W,
the Xh residue in SEQ ID NO: 23 is Y,
the X1 residue in SEQ ID NO: 24 is D,
the X2 residue in SEQ ID NO: 24 is A,
the X4 residue in SEQ ID NO: 25 is G,
the X5 residue in SEQ ID NO: 25 is N,
the X6 residue in SEQ ID NO: 26 is A,
the X7 residue in SEQ ID NO: 26 is A,
the X8 residue in SEQ ID NO: 26 is S, and
the X9 residue in SEQ ID NO: 26 is S.

24. The anti-TrkA antibody, or antigen-binding fragment thereof, of claim 20, comprising:
(a) a heavy chain variable region (HCVR) CDR1, CDR2 and CDR3 amino acid sequences derived from an amino acid sequence of SEQ ID NO: 27, 29, 33, 35, 37, 39, 41, 43, 45, 47, 49, 51, 53, 57, 59, 61, 63 or 65, and
(b) a light chain variable region (LCVR) CDR1, CDR2 and CDR3 amino acid sequences derived from an amino acid sequence of SEQ ID NO: 28, 30, 32, 34, 36, 38, 40, 42, 44, 46, 48, 50, 52, 54, 56, 58, 60, 62, 64 or 66,
wherein the CDR sequences are identified by the Kabat, Chothia, AbM or IMGT numbering schemes, or combinations thereof.

25. The anti-TrkA antibody, or antigen-binding fragment thereof, of claim 20, wherein TrkA activation is measured by one or more of the following:
neurite outgrowth,
dimerization of TrkA,
internalization of TrkA,
phosphorylation of TrkA, or
TrkA signaling (including for example, activation of an intracellular pathways associated with neurite outgrowth and neurite survival, including MAPK pathway and P13K pathways (preferably, as measured by assessing Erk and/or Akt phosphorylation)).

26. The anti-TrkA antibody, or antigen-binding fragment thereof, of claim 25, wherein the antibody, or antigen-binding fragment thereof, induces neurite outgrowth in the absence of NGF.

27. The anti-TrkA antibody, or antigen-binding fragment thereof, of claim 20, wherein the antibody, or antigen-binding fragment thereof, induces a submaximal TrkA biological response and/or submaximal induction of one or more downstream pathways in the absence of NGF (Ab-NGF), when compared to NGF induced activation of TrkA measured in the absence of the antibody (NGF-Ab).

28. The anti-TrkA antibody, or antigen-binding fragment thereof, of claim 27, wherein the antibody, or antigen-binding fragment thereof, induces TrkA activation in the absence of NGF (antibody (Ab)-NGF), when compared to a control of NGF induced TrkA activation when measured in the absence of the antibody (NGF-Ab) by at least about 45%, 50%, 55%, 60%, 65%, 70%, 75%, or 80% up to about 85%.

29. The anti-TrkA antibody, or antigen-binding fragment thereof, of claim 20, wherein the antibody has an EC₅₀ value from about 50 μM to about 100 μM, preferably from about 70 μM to 90 μM as determined by an in vitro neurite outgrowth assay comprising PC12 cells, when compared to a control of NGF absent antibody having an EC₅₀ value from about 1 μM to about 15 μM, preferably from about 5 μM to about 8 μM.

30. The anti-TrkA antibody, or antigen-binding fragment thereof, of claim 20, wherein the antibody is a human antibody, preferably wherein the humanness score of the anti-TrkA antibody is at least about 90% or 95% up to about 98%.

31. The anti-TrkA antibody, or antigen-binding fragment thereof, of claim 20, wherein the antibody is an IgG1, an IgG2, an IgG3, an IgG4, an IgM, an IgA1, an IgA2, a secretory IgA, an IgD, or an IgE antibody, preferably an IgG1, IgG2 or IgG4 antibody.

32. The anti-TrkA antibody, or antigen-binding fragment thereof, of claim 20, comprising:
(a) a heavy chain constant region amino acid sequence of one of SEQ ID NO: 1-18, and
(b) a light chain constant region amino acid sequence of SEQ ID NO: 19 or 20.

33. The anti-TrkA antibody, or antigen-binding fragment thereof, of claim 20, which exhibits one or more of the following characteristics:
(i) a dissociation constant (KD) for binding human TrkA ranging from about 0.1 nM to about 1,000 nM, preferably about 0.1 nM to about 10 nM, as determined by surface plasmon resonance; and/or
(ii) a half-maximal effective concentration (EC₅₀) value for binding human TrkA ranging from about 10 nM to about 1,000 nM, preferably about 100 nM to about 500 nM.

34. The anti-TrkA antibody, or antigen-binding fragment thereof, of claim 20, comprising:
(a) a heavy chain variable region (HCVR) sequence of SEQ ID NO: 27, 29, 33, 35, 37, 39, 41, 43, 45, 47, 49, 51, 53, 57, 59, 61, 63 or 65, and
(b) a light chain variable region (LCVR) of SEQ ID NO:28, 30, 32, 34, 36, 38, 40, 42, 44, 46, 48, 50, 52, 54, 56, 58, 60, 62, 64 or 66.

35. The anti-TrkA antibody, or antigen-binding fragment thereof, of claim 34, comprising:
(a) a heavy chain variable region (HCVR) sequence of SEQ ID NO: 27, 29 or 49, and
(b) a light chain variable region (LCVR) of SEQ ID NO: 28, 30 or 50.

36. The anti-TrkA antibody, or antigen-binding fragment thereof, of claim 20, wherein the antibody, or antigen-binding fragment thereof, is a monoclonal antibody.

37. The anti-TrkA antibody, or antigen-binding fragment thereof, of claim 20, wherein the antibody, or antigen-binding fragment thereof, is a human antibody.

38. A composition comprising the anti-TrkA antibody, or antigen-binding fragment thereof, of claim 20, and a pharmaceutically acceptable carrier.

39. A nucleic acid molecule encoding the anti-TrkA antibody, or antigen-binding fragment thereof, of claim 20.

40. An expression vector comprising the nucleic acid molecule of claim 39.

41. A host cell comprising the expression vector of claim 40.

42. A method of promoting neurite regeneration in a subject in need thereof or treating disorders in which neuronal dysfunction is associated with the disease pathology in a subject in need thereof, comprising administering an effective amount of the anti-TrkA antibody, or antigen-binding fragment thereof, of claim 20, wherein the anti-TrkA antibody binds to and activates TrkA thereby treating the pathology.


SEQUENCE LISTING (78 sequences)

SEQ ID NO: 1 - protein, 329 aa, Homo sapiens
  source 1..329: note = heavy chain constant region sequence, human IgG1
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 103 aa, synthetic construct
  source 1..103: note = heavy chain constant region sequence, human IgG1 FAB TAG
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 3 - protein, 329 aa, synthetic construct
  source 1..329: note = heavy chain constant region sequence, human IgG1 KiH Hole
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 4 - protein, 329 aa, synthetic construct
  source 1..329: note = Human IgG1 KiH Knob
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 5 - protein, 329 aa, synthetic construct
  source 1..329: note = heavy chain constant region sequence, Human IgG1 (L235A, G237A)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 6 - protein, 329 aa, synthetic construct
  source 1..329: note = heavy chain constant region sequence, human IgG1 YTE
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 7 - protein, 329 aa, Homo sapiens
  source 1..329: note = Human IgG1-LALA-PG (L234A/L235A/P329G)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPESTRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 8 - protein, 329 aa, Homo sapiens
  source 1..329: note = Human IgG1-STR (L234S/L235T/G236R)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 9 - protein, 325 aa, synthetic construct
  source 1..325: note = heavy chain constant region sequence, human IgG2DASS
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 10 - protein, 326 aa, Homo sapiens
  source 1..326: note = heavy chain constant region sequence, human IgG4
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 11 - protein, 326 aa, synthetic construct
  source 1..326: note = heavy chain constant region sequence, human IgG4 KiH Hole
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLG

SEQ ID NO: 12 - protein, 326 aa, synthetic construct
  source 1..326: note = heavy chain constant region sequence, human IgG4 KiH Knob
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFAGAPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 13 - protein, 326 aa, synthetic construct; identical to SEQ ID NO: 12
  source 1..326: note = heavy chain constant region sequence, human IgG4 (L235A, G237A)

SEQ ID NO: 14 - protein, 326 aa, synthetic construct
  source 1..326: note = heavy chain constant region sequence, human IgG4 (L235E)
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 15 - protein, 326 aa, synthetic construct
  source 1..326: note = heavy chain constant region sequence, human IgG4 YTE
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 16 - protein, 326 aa, synthetic construct
  source 1..326: note = heavy chain constant region sequence, human IgG4 YTE KiH Hole
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLG

SEQ ID NO: 17 - protein, 326 aa, synthetic construct
  source 1..326: note = heavy chain constant region sequence, human IgG4 YTE KiH Knob
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 18 - protein, 329 aa, synthetic construct
  source 1..329: note = heavy chain constant region sequence, human IgG1 (L235A, G237A), YTE
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 19 - protein, 107 aa, Homo sapiens [sequence omitted]

SEQ ID NO: 20 - protein, 106 aa, Homo sapiens
  source 1..106: note = Human lambda, light chain constant region sequence
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 21 - protein, 11 aa, synthetic construct
  VARIANT 4: note = X at position 4 is denoted Xb and corresponds to H29 (Kabat) in the heavy chain variable region. Xb = V or I
  VARIANT 7: note = X at position 7 is denoted Xc and corresponds to H32 (Kabat) in the heavy chain variable region. Xc = G or S.
GGSXSSXNWWT

SEQ ID NO: 22 - protein, 16 aa, synthetic construct
  VARIANT 8: note = X at position 8 is denoted Xd and corresponds to heavy chain variable region H57 [Kabat]. Xd = T or R.
EIYHRGNXNYNPSLKS

SEQ ID NO: 23 - protein, 6 aa, synthetic construct
  VARIANT 3: note = X at position 3 is denoted Xg and corresponds to heavy chain variable region H97 (Kabat). Xg = F or W.
  VARIANT 6: note = X at position 6 is denoted Xh and corresponds to heavy chain variable region H102 (Kabat). Xh = Y or R.
APXFDX

SEQ ID NO: 24 - protein, 11 aa, synthetic construct
  VARIANT 3: note = X at position 3 is denoted X1 and corresponds to light chain variable region L26 [Kabat]. X1 = D, G, A or L.
  VARIANT 4: note = X at position 4 is denoted X2 and corresponds to light chain variable region L27 [Kabat]. X2 = S or A.
QGXXLRSYYAS

SEQ ID NO: 25 - protein, 7 aa, synthetic construct
  VARIANT 3: note = X at position 3 is denoted X4 and corresponds to light chain variable region L52 [Kabat]. X4 = N or G.
  VARIANT 4: note = note = X at position 4 is denoted X5 and corresponds to light chain variable region L53 [Kabat]. X5 = N or K.
GKXXRPS

SEQ ID NO: 26 - protein, 11 aa, synthetic construct
  VARIANT 1: note = X at position 1 is denoted X6 and corresponds to light chain variable region L89 [Kabat]. X6 = N or A.
  VARIANT 4: note = X at position 4 is denoted X7 and corresponds to light chain variable region L92 [Kabat]. X7 = V, D or A.
  VARIANT 5: note = X at position 5 is denoted X8 and corresponds to light chain variable region L93 [Kabat]. X8 = S or Y.
  VARIANT 6: note = note = X at position 6 is denoted X9 and corresponds to light chain variable region L94 [Kabat]. X9 = G or S.
XSQXXXGNHLI

SEQ ID NO: 27 - protein, 111 aa, synthetic construct
  source 1..111: note = Ab3 heavy chain variable region
QVQLQESGPGLVKPSGTLSLTCAVSGGSVSSGNWWTWVRQPPGKGLEWIGEIYHRGNTNYNPSLKSRVTISVDKSKNQFSLKLNSVTAADTAVYFCARAPFFDYWGQGTLV

SEQ ID NO: 28 - protein, 109 aa, synthetic construct
  source 1..109: note = Ab3 light chain variable region
SSELTQDPAVSVALGQTVRITCQGDSLRSYYASWYQQKPGQAPVLVIFGKNNRPSGIPDRFSGSSSGNTASLTITGAQAEDEADYYCNSQVSGGNHLIFGGGTKLTVLG

SEQ ID NO: 29 - protein, 111 aa, synthetic construct
  source 1..111: note = Ab386 heavy chain variable region
QVQLQESGPGLVKPSGTLSLTCAVSGGSISSSNWWTWVRQPPGKGLEWIGEIYHRGNRNYNPSLKSRVTISVDKSKNQFSLKLSSVTAADTAVYYCARAPWFDYWGQGTLV

SEQ ID NO: 30 - protein, 109 aa, synthetic construct
  source 1..109: note = Ab386 light chain variable region
SSELTQDPAVSVALGQTVRITCQGDSLRSYYASWYQQKPGQAPVLVIYGKNNRPSGIPDRFSGSSSGNTASLTITGAQAEDEADYYCNSQDSSGNHLIFGGGTKLTVLG

SEQ ID NO: 31 - protein, 111 aa, synthetic construct
  source 1..111: note = Ab449 heavy chain variable region
QVQLQESGPGLVKPSGTLSLTCAVSGGSISSSNWWTWVRQPPGKGLEWIGEIYHRGNRNYNPSLKSRVTISVDKSKNQFSLKLSSVTAADTAVYYCARAPWFDRWGQGTLV

SEQ ID NO: 32 - protein, 109 aa, synthetic construct; identical to SEQ ID NO: 30
  source 1..109: note = Ab449 light chain variable region

SEQ ID NO: 33 - protein, 111 aa, synthetic construct
  source 1..111: note = Ab476 heavy chain variable region
QVQLQESGPGLVKPSGTLSLTCAVSGGSISSSNWWTWVRQPPGKGLEWIGEIYHRGNRNYNPSLKSRVTISVDKSKNQFSLKLSSVTAADTAVYYCARAPFFDRWGQGTLV

SEQ ID NO: 34 - protein, 109 aa, synthetic construct
  source 1..109: note = Ab476 light chain variable region
SSELTQDPAVSVALGQTVRITCQGDSLRSYYASWYQQKPGQAPVLVIHGKNNRPSGIPDRFSGSSSGNTASLTITGAQAEDEADYYCNSQDSSGNHLIFGGGTKLTVLG

SEQ ID NO: 35 - protein, 111 aa, synthetic construct
  source 1..111: note = Ab523 heavy chain variable region
QVQLQESGPGLVKPSGTLSLTCAVSGGSVSSGNWWTWVRQPPGKGLEWIGEIYHRGNRNYNPSLKSRVTISVDKSKNQFSLKLNSVTAADTAVYFCARAPFFDYWGQGTLV

SEQ ID NO: 36 - protein, 109 aa, synthetic construct
  source 1..109: note = Ab523 light chain variable region
SSELTQDPAVSVALGQTVRITCQGDSLRSYYASWYQQKPGQAPVLVIYGKNNRPSGIPDRFSGSSSWNTASLTITGAQAEDEADYYCNSQDSSGNHLIFGGGTKLTVLG

SEQ ID NO: 37 - protein, 111 aa, synthetic construct; identical to SEQ ID NO: 27
  source 1..111: note = Ab676 heavy chain variable region

SEQ ID NO: 38 - protein, 120 aa, synthetic construct
  source 1..120: note = Ab676 light chain variable region
SSELTQDPAVSVALGQTVRITCQGDSLRSYYASWYQQKPGQAPVLVIHGKNNRPSGIPDRFSGSSSGNTASLTITGAQAEDEADYYCNSQVYGGNHLIFGGGTKLTVLGQGDSLRSYYAS

SEQ ID NO: 39 - protein, 111 aa, synthetic construct
  source 1..111: note = Ab49 heavy chain variable region
EVQLQESGPGLVKPSGTLSLTCAVSGGSISSSNWWTWVRQPPGKGLEWIGEIYHRGNRNYNPSLKSRVTISVDKSKNQFSLKLSSVTAADTAVYYCARAPWFDYWGQGTLV

SEQ ID NO: 40 - protein, 106 aa, synthetic construct
  source 1..106: note = Ab49 light chain variable region
SSELTQDPAVSVALGQTVRITCQGDSLRSYYASWYQQKPGQAPVLVIYGKNNRPSGIPDRFSGSSSGNTASLTITGAQAEDEADYYCASQASSGNHLIFGGGTKLT

SEQ ID NO: 41 - protein, 111 aa, synthetic construct; identical to SEQ ID NO: 39
  source 1..111: note = Ab67 heavy chain variable region

SEQ ID NO: 42 - protein, 106 aa, synthetic construct
  source 1..106: note = Ab67 light chain variable region
SSELTQDPAVSVALGQTVRITCQGDALRSYYASWYQQKPGQAPVLVIYGKNNRPSGIPDRFSGSSSGNTASLTITGAQAEDEADYYCNSQASSGNHLIFGGGTKLT

SEQ ID NO: 43 - protein, 111 aa, synthetic construct; identical to SEQ ID NO: 39
  source 1..111: note = Ab70 heavy chain variable region

SEQ ID NO: 44 - protein, 106 aa, synthetic construct
  source 1..106: note = Ab70 light chain variable region
SSELTQDPAVSVALGQTVRITCQGDALRSYYASWYQQKPGQAPVLVIYGKNNRPSGIPDRFSGSSSGNTASLTITGAQAEDEADYYCASQASSGNHLIFGGGTKLT

SEQ ID NO: 45 - protein, 111 aa, synthetic construct; identical to SEQ ID NO: 39
  source 1..111: note = Ab76 heavy chain variable region

SEQ ID NO: 46 - protein, 106 aa, synthetic construct
  source 1..106: note = Ab76 light chain variable region
SSELTQDPAVSVALGQTVRITCQGDALRSYYASWYQQKPGQAPVLVIYGKNKRPSGIPDRFSGSSSGNTASLTITGAQAEDEADYYCASQASSGNHLIFGGGTKLT

SEQ ID NO: 47 - protein, 111 aa, synthetic construct; identical to SEQ ID NO: 39
  source 1..111: note = Ab79 heavy chain variable region

SEQ ID NO: 48 - protein, 106 aa, synthetic construct
  source 1..106: note = Ab79 light chain variable region
SSELTQDPAVSVALGQTVRITCQGDALRSYYASWYQQKPGQAPVLVIYGKGNRPSGIPDRFSGSSSGNTASLTITGAQAEDEADYYCNSQASSGNHLIFGGGTKLT

SEQ ID NO: 49 - protein, 111 aa, synthetic construct; identical to SEQ ID NO: 29
  source 1..111: note = Ab82 heavy chain variable region

SEQ ID NO: 50 - protein, 106 aa, synthetic construct
  source 1..106: note = Ab82 light chain variable region
SSELTQDPAVSVALGQTVRITCQGDALRSYYASWYQQKPGQAPVLVIYGKGNRPSGIPDRFSGSSSGNTASLTITGAQAEDEADYYCASQASSGNHLIFGGGTKLT

SEQ ID NO: 51 - protein, 111 aa, synthetic construct; identical to SEQ ID NO: 39
  source 1..111: note = Ab96 heavy chain variable region

SEQ ID NO: 52 - protein, 106 aa, synthetic construct
  source 1..106: note = Ab96 light chain variable region
SSELTQDPAVSVALGQTVRITCQGGSLRSYYASWYQQKPGQAPVLVIYGKNKRPSGIPDRFSGSSSGNTASLTITGAQAEDEADYYCNSQASSGNHLIFGGGTKLT

SEQ ID NO: 53 - protein, 111 aa, synthetic construct; identical to SEQ ID NO: 39
  source 1..111: note = Ab101 heavy chain variable region

SEQ ID NO: 54 - protein, 106 aa, synthetic construct
  source 1..106: note = Ab101 light chain variable region
SSELTQDPAVSVALGQTVRITCQGGSLRSYYASWYQQKPGQAPVLVIYGKGNRPSGIPDRFSGSSSGNTASLTITGAQAEDEADYYCNSQDSSGNHLIFGGGTKLT

SEQ ID NO: 55 - protein, 111 aa, synthetic construct; identical to SEQ ID NO: 39
  source 1..111: note = Ab119 heavy chain variable region

SEQ ID NO: 56 - protein, 106 aa, synthetic construct
  source 1..106: note = Ab119 light chain variable region
SSELTQDPAVSVALGQTVRITCQGASLRSYYASWYQQKPGQAPVLVIYGKNKRPSGIPDRFSGSSSGNTASLTITGAQAEDEADYYCNSQASSGNHLIFGGGTKLT

SEQ ID NO: 57 - protein, 111 aa, synthetic construct; identical to SEQ ID NO: 39
  source 1..111: note = Ab122 heavy chain variable region

SEQ ID NO: 58 - protein, 106 aa, synthetic construct
  source 1..106: note = Ab122 light chain variable region
SSELTQDPAVSVALGQTVRITCQGASLRSYYASWYQQKPGQAPVLVIYGKNKRPSGIPDRFSGSSSGNTASLTITGAQAEDEADYYCASQASSGNHLIFGGGTKLT

SEQ ID NO: 59 - protein, 111 aa, synthetic construct; identical to SEQ ID NO: 39
  source 1..111: note = Ab124 heavy chain variable region

SEQ ID NO: 60 - protein, 106 aa, synthetic construct
  source 1..106: note = Ab124 light chain variable region
SSELTQDPAVSVALGQTVRITCQGASLRSYYASWYQQKPGQAPVLVIYGKGNRPSGIPDRFSGSSSGNTASLTITGAQAEDEADYYCNSQDSSGNHLIFGGGTKLT

SEQ ID NO: 61 - protein, 111 aa, synthetic construct; identical to SEQ ID NO: 39
  source 1..111: note = Ab142 heavy chain variable region

SEQ ID NO: 62 - protein, 106 aa, synthetic construct
  source 1..106: note = Ab142 light chain variable region
SSELTQDPAVSVALGQTVRITCQGLSLRSYYASWYQQKPGQAPVLVIYGKNKRPSGIPDRFSGSSSGNTASLTITGAQAEDEADYYCNSQASSGNHLIFGGGTKLT

SEQ ID NO: 63 - protein, 111 aa, synthetic construct; identical to SEQ ID NO: 39
  source 1..111: note = Ab145 heavy chain variable region

SEQ ID NO: 64 - protein, 106 aa, synthetic construct
  source 1..106: note = Ab145 light chain variable region
SSELTQDPAVSVALGQTVRITCQGLSLRSYYASWYQQKPGQAPVLVIYGKNKRPSGIPDRFSGSSSGNTASLTITGAQAEDEADYYCASQASSGNHLIFGGGTKLT

SEQ ID NO: 65 - protein, 111 aa, synthetic construct; identical to SEQ ID NO: 39
  source 1..111: note = Ab147 heavy chain variable region

SEQ ID NO: 66 - protein, 106 aa, synthetic construct
  source 1..106: note = Ab147 light chain variable region
SSELTQDPAVSVALGQTVRITCQGLSLRSYYASWYQQKPGQAPVLVIYGKGNRPSGIPDRFSGSSSGNTASLTITGAQAEDEADYYCNSQDSSGNHLIFGGGTKLT

SEQ ID NO: 67 - protein, 98 aa, Homo sapiens
  source 1..98: note = IGHV4-4*02 heavy chain
QVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCAR

SEQ ID NO: 68 - protein, 96 aa, Homo sapiens
  source 1..96: note = IGLV3-19*01 light chain
SSELTQDPAVSVALGQTVRITCQGDSLRSYYASWYQQKPGQAPVLVIYGKNNRPSGIPDRFSGSSSGNTASLTITGAQAEDEADYYCNSRDSSGNH

SEQ ID NO: 69 - protein, 111 aa, Homo sapiens
  source 1..111: note = GL3 variant heavy chain
QVQLQESGPGLVKPSGTLSLTCAVSGGSISSSNWWTWVRQPPGKGLEWIGEIYHRGNTNYNPSLKSRVTISVDKSKNQFSLKLSSVTAADTAVYYCARAPFFDYWGQGTLV

SEQ ID NO: 70 - protein, 109 aa, Homo sapiens; identical to SEQ ID NO: 30
  source 1..109: note = GL3 variant light chain

SEQ ID NO: 71 - protein, 11 aa, synthetic construct
GGSISSSNWWT

SEQ ID NO: 72 - protein, 16 aa, synthetic construct
EIYHRGNRNYNPSLKS

SEQ ID NO: 73 - protein, 6 aa, synthetic construct
APWFDY

SEQ ID NO: 74 - protein, 11 aa, synthetic construct
QGDALRSYYAS

SEQ ID NO: 75 - protein, 7 aa, synthetic construct
GKGNRPS

SEQ ID NO: 76 - protein, 11 aa, synthetic construct
ASQASSGNHLI

SEQ ID NO: 77 - protein, 445 aa, synthetic construct
  VARIANT 445: note = deletion of the c-terminal lysine (K)
QVQLQESGPGLVKPSGTLSLTCAVSGGSISSSNWWTWVRQPPGKGLEWIGEIYHRGNRNYNPSLKSRVTISVDKSKNQFSLKLSSVTAADTAVYYCARAPWFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 78 - protein, 214 aa, synthetic construct
SSELTQDPAVSVALGQTVRITCQGDALRSYYASWYQQKPGQAPVLVIYGKGNRPSGIPDRFSGSSSGNTASLTITGAQAEDEADYYCASQASSGNHLIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS